SEQUENCE LISTING (272 sequences)

SEQ ID NO: 1 - protein, 124 aa, Artificial Sequence
  Variable heavy chain (VH) Jovi-1 VH
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSS

SEQ ID NO: 2 - protein, 113 aa, Artificial Sequence
  Variable light chain (VL) Jovi-1 VL
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 3 - protein, 252 aa, Artificial Sequence
  Jovi-1 scFv
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSGGGGSGGGGSGGGGSDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 4-6 - skipped (3 entries)

SEQ ID NO: 7 - protein, 7 aa, Artificial Sequence
  Complementarity determining region (CDR) variable heavy chain (VH) CDR1
GYTFTGY

SEQ ID NO: 8 - protein, 6 aa, Artificial Sequence
  VH CDR2
NPYNDD

SEQ ID NO: 9 - protein, 15 aa, Artificial Sequence
  VH CDR3
GAGYNFDGAYRFFDF

SEQ ID NO: 10 - protein, 16 aa, Artificial Sequence
  Variable light chain (VL) CDR1
RSSQRLVHSNGNTYLH

SEQ ID NO: 11 - protein, 7 aa, Artificial Sequence
  VL CDR2
RVSNRFP

SEQ ID NO: 12 - protein, 9 aa, Artificial Sequence
  VL CDR3
SQSTHVPYT

SEQ ID NO: 13 - protein, 247 aa, Artificial Sequence
  Single chain fragment variable (scFv) CP_01_E09
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARAHNSSSWSFDYWGQGTLVTVSSGGGGSGGGGSGGGASDIQMTQSPSSLSASVGDRVTITCRASQSISSYLNWYQQKPGKAPKLLIYDASNLETGVPSRFSGSGSGTDFTFTISSLQPEDIATYYCQQYDNLPLTFGGGTKVDIKRTAAA

SEQ ID NO: 14 - protein, 246 aa, Artificial Sequence
  scFv CP_01_D12
EVQLLESGGGVVQPGRSLRLSCAASGFTFSSYAMHWVRQAPGKGLEWVAVISYDGSNKYYADSVKGRFTISRDNSKNTLYLQMNSLRAEDTAVYYCAKGGDTYGFLDNWGQGTMVTVSSGGGGSGGGGSGGGASDIQMTQSPSTLSASVGDRVTITCRASQSISSWLAWYQQKPGKAPKLLIYKASSLESGVPSRFSGSGSGTDFTLTISSLQPEDFATYYCQQFNAYPLTFGGGTKVEIKRTAAA

SEQ ID NO: 15 - protein, 246 aa, Artificial Sequence
  scFv CP_01_D10
QVQLVESGGGLVQPGRSLRLSCAASGFTFSSYAMHWVRQAPGKGLEWVAVISYDGSSKYYADSVKGRFTISRDNSKNTLYLQMNSLRAEDTAVYYCAKGGGSFGAFDIWGQGTLVTVSSGGGGSGGGGSGGGASDIQMTQSPSSLSASVGDRVTITCRASQSISRYLNWYQQKPGKAPNLLIYAASSLQSGVPSRFSGSGSGTDFTLTISSLQPEDFATYYCQQYNSYPLTFGGGTKLEIKRTAAA

SEQ ID NO: 16 - protein, 246 aa, Artificial Sequence
  scFv CP_01_C08
EVQLLESGGGAVQPGRSLRLSCAASGFTFSSYAMHWVRQAPGKGLEWVAVISYDGSNKYYADSVKGRFTISRDNSKNTLYLQMNSLRAEDTAVYYCASGYSSSWYLDYWGQGTLVTVSSGGGGSGGGGSGGGASDIQMTQSPSSVSASVGDRVTITCQASQDISNYLNWYQQKPGKAPKLLIYDASNLETGVPSRFSGSGSGTDFTFTISSLQPEDIATYYCQQYDNLPLTFGGGTKVEIKRTAAA

SEQ ID NO: 17 - protein, 245 aa, Artificial Sequence
  scFv CP_01_C11
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCASGGAGWNWGQGTMVTVSSGGGGSGGGGSGGGASNFMLTQPHSVSESPGKTATISCTRSSGSIASNYVQWYQQRPGSAPTTVIYEDNQRPFGVPDRFSGSIDSSSNSASLTISGLKTEDEADYYCQSHDSSNVVFGGGTQLTVLGQPAA

SEQ ID NO: 18 - protein, 247 aa, Artificial Sequence
  scFv CP_01_F03
  misc_feature (101)..(101): Xaa can be any naturally occurring amino acid
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGYXASSWSQGLWGQGTLVTVSSGGGGSGGGGSGGGASDIQMTQSPSSLSASVRDRVTITCQASQDISNYLNWYQQKPGKAPKLLIYDASNLETGVPSRFSGSGSGTDFTFTISSLQPEDIATYYCQQYDNLPPTFGGGTKVEIKRTAAA

SEQ ID NO: 19 - protein, 251 aa, Artificial Sequence
  scFv CP_01_E07
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDLGGSGGAFDIWGQGTLVTVSSGGGGSGGGGSGGGASDIVMTQTPHSLSVTPGQPASISCKSSQSLLYSDGKTYLYWYLQKPGQPPQLLIYEVSNRFSGVPDRFSGSGSGTDFTLKISRVEAEDVGVYYCMQSIQLYTFGQGTKVDIKRTAAA

SEQ ID NO: 20 - protein, 244 aa, Artificial Sequence
  scFv CP_01_D03
QVQLVESGGGVVQPGRSLRLSCAAPGFTFSSYGMHWVRQAPGKGLEWVAVISYDGSNKYYADSVKGRFTISRDNSKNTLYLQMNSLRAEDTAVYYCARNKQYGMDVWGQGTLVTVSSGGGGSGGGGSGGGASDIVMTQSPATLSLAPGERATLSCRASQSVGSNLAWYQQKPGQAPSLLIYDASTRATGIPARFSGSGSGTDFTLTISSLQSEDIAVYYCQQYHRWPLTFGGGTKVEIKRTAAA

SEQ ID NO: 21 - protein, 249 aa, Artificial Sequence
  scFv CP_01_F06
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDDGAMRYWGQGTMVTVSSGGGGSGGGGSGGGASDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYDSPYTFGQGTKVDIKRTAAA

SEQ ID NO: 22 - protein, 244 aa, Artificial Sequence
  scFv CP_01_F02
QVQLVESGGGVVQPGRPLRLSCAASGFTFSSYAMHWVRQAPGKGLEWVAVISYDGSNKYYADSVKGRFTISRDNSKNTLYLQMNSLRAEDTAVYYCARAGYSYADYWGQGTMVTVSSGGGGSGGGGSGGGASDIQMTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVDIKRTAAA

SEQ ID NO: 23 - protein, 249 aa, Artificial Sequence
  scFv CP_03_E05
EVQLVESGGGVVQPGGSLRLSCAASGFTFSSYAMSWVRQAPGKGLEWVSAISGSGGSTYYADSVKGRFSISRDNSKNTLYLQMNSLRAEDTAVYYCARTRSSGAFDIWGQGTLVTVSSGGGGSGGGGSGGGASNFMLTQPHSVSESPGKTVTISCTRSSGSIASKYVQWYQQRPGSSPTTVIYEDNQRPSGVPDRFSGSIDTSSNSASLTISGLRTEDEADYYCHSYDSNNHSVFGGGTKVTVLGQPAA

SEQ ID NO: 24 - protein, 246 aa, Artificial Sequence
  scFv CP_03_D05
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCASPRGRGSAFDIWGQGTLVTVSSGGGGSGGGGSGGGASSYELTQPPSVSVSPGQTATISCSGDQLGGKYGHWYQKKPGQSPVLVLYQDRKRPAGIPERFSGSSSGNTITLTISGTQAVDEADYYCQAWDTNLGGVFGGGTKVTVLGQPAA

SEQ ID NO: 25 - protein, 249 aa, Artificial Sequence
  scFv CP_03_H06
QVQLVESGGGLVKPGGSLRLSCAASGFTFSDYYMSWIRQAPGKGLEWVSYISSSGSTIYYADSVEGRFTISRDNAKNSLYLQMNSLRTEDTAVYYCARARVGGMDVWGQGTMVTVSSGGGGSGGGGSGGGASNFMLTQPHSVSESPGKTVTISCTRSSGSIASNYVQWYQQRPGSSPTTVIYEDNQRPSGVPDRFSGSIDSSSNSASLTISGLKTEDEADYYCQSFDADNLHVVFGGGTKLTVLGQPAA

SEQ ID NO: 26 - protein, 248 aa, Artificial Sequence
  scFv CP_03_C12
EVQLVQSGAEVKKPGASVKVSCKASGYTFTGYYMHWVRQAPGQGLEWMGWINPNSGGTNYAQKFQGRVTMTRDTSISTAYMELSSLRSEDTAVYYCARDTGPIDYWGQGTMVTVSSGGGGSGGGGSGGGASDIVMTQTPLSLSVTPGQPASISCKSSQSLLHSDGKTYLYWYLQKPGQPPQLLVYEVSNRFSGVPDKFSGSGSGTDFTLKISRVEAEDVGVYYCMQGIQLPPTFGGGTKVDIKRTAAA

SEQ ID NO: 27 - protein, 249 aa, Artificial Sequence
  scFv CP_03_G02
EVQLVQSGAEVKKPGSSVKVSCKASGGTFSSYAISWVRQAPGQGLEWMGIINPSGGSTSYAQKFQGRVTMTRDTSTSIVYMELSSLRSEDTAVYYCARGVWNSGSYLGFDYWGQGTLVTVSSGGGGSGGGGSGGGASDIQMTQSPSSLSASVGDRVTITCQASQDISNYLNWYQQKPGKAPKLLIYAASSLQSGVPSRFSGSGSGTDFTLTISSLQPEDFATYYCQQSYSTPLTFGGGTKLEIKRTAAA

SEQ ID NO: 28 - protein, 251 aa, Artificial Sequence
  scFv CP_03_D04
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGGFTVPGGAFDIWGQGTLVTVSSGGGGSGGGGSGGGASNFMLTQPHSVSDSPGKTVTISCTRSSGRIGSNFVQWYQQRPGSSPTTVIYEDDQRPSGVPARFSGSIDSSSNSASLTISGLTTADEAGYYCQSYDASNVIFGGGTKLTVLGQPAA

SEQ ID NO: 29 - protein, 247 aa, Artificial Sequence
  scFv CP_03_F10
EVQLVESGAEVKKPGASVKVSCKASGYTFTGYYMHWVRQAPGQGLEWMGRINPNSGGTNYAQKFQGRVTMTRDTSISTAYMELSSLRSEDTAVYYCARFGERYAFDIWGQGTLVTVSSGGGGSGGGGSGGGASQSELTQPPSASGSPGQSVTISCTGTSTDVGAFHFVSWYQHTPGKAPKLLISEVRKRASGVPDRFSGSRSGNTASLTVSGLQSEDEADYFCSAYTGSNYVFGSGTKLTVLGQPAA

SEQ ID NO: 30 - protein, 252 aa, Artificial Sequence
  scFv CP_03_G09
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDQWLANYYYYGMDVWGQGTLVTVSSGGGGSGGGGSGGGASSYELTQPLSVSVALGQTARITCGGNNIGSKNVHWYQQKPGQAPVLVIYRDNNRPSGIPERFSGSNSGNTATLTISKAQAGDEADYYCQVWDSNSWVFGGGTKLTVLGQPAA

SEQ ID NO: 31 - protein, 252 aa, Artificial Sequence
  scFv CP_03_F09
QMQLVQSGAEVKKPGASVKVSCKASGYTFASYYMHWVRQAPGQGLEWMGIINPSGGSTSYAQKFQGRVTMTRDTSTSTVYMELSRLRSDDTAVYYCASNRGGSYKSVGMDVWGQGTTVTVSSGGGGSGGGGSGGGASNFMLTQPQSVSESPGKTVTISCTRSSGNFASKYVQWYQQRPGSSPTTVIYENYQRPSGVPDRFSGSIDSSSNSATLTISGLKTEDEADYYCQSYDEVSVVFGGGTQLTVLGQPAA

SEQ ID NO: 32 - protein, 249 aa, Artificial Sequence
  scFv CP_03_D09
EVQLVQSGAEVKKPGSSVKVSCEASGYTFTSYAISWVRQAPGQGLEWMGWMNPNSGNTGYAQKFQGRVTMTRNTSISTAYMELSSLRSEDTAVYYCARVSSYYGMDVWGQGTLVTVSSGGGGSGGGGSGGGASNFMLTQPLSVSESPGKTVTISCTRSSGSIASNYVQWYQQRPGSAPTTVIYEDNQRPSGVPDRFSGSIDSSSNSASLTISGLKTEDEADYYCQSYNSSNHWVFGGGTKVTVLGQPAA

SEQ ID NO: 33 - protein, 537 aa, Artificial Sequence
  Chimeric antigen receptor (CAR), JOVI-1 CAR with CD8 stalk spacer
METDTLLLWVLLVWIPGSTGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSGGGGSGGGGSGGGGSDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 727 aa, Artificial Sequence
  JOVI-1 CAR with H-CH2-CH3pvaa spacer
METDTLLLWVLLVWIPGSTGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSGGGGSGGGGSGGGGSDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 511 aa, Artificial Sequence
  JOVI-1 CAR with IgG1 hinge spacer
METDTLLLWVLLVWIPGSTGEVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSSGGGGSGGGGSGGGGSDVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRSDPAEPKSPDKTHTCPPCPKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 15 aa, Artificial Sequence
  Synthetic peptide for TRBC2
VLEDLKNVFPPEVAV

SEQ ID NO: 37 - protein, 15 aa, Artificial Sequence
  Synthetic peptide for TRBC1
VLEDLNKVFPPEVAV

SEQ ID NO: 38 - protein, 16 aa, Artificial Sequence
  Synthetic peptide for TRBC1
VLEDLNKVFPPEVAVC

SEQ ID NO: 39 - protein, 16 aa, Artificial Sequence
  Synthetic peptide for TRBC2
VLEDLKNVFPPEVAVC

SEQ ID NO: 40 - protein, 175 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKD

SEQ ID NO: 41 - protein, 175 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKD

SEQ ID NO: 42 - protein, 9 aa, Artificial Sequence
  Heavy CDR3 Clone CP_01_E09
AHNSSSWSF

SEQ ID NO: 43 - protein, 8 aa, Artificial Sequence
  Heavy CDR3 Clone CP_01_D12
GGDTYGFL

SEQ ID NO: 44 - protein, 8 aa, Artificial Sequence
  Heavy CDR3 Clone CP_01_D10
GGGSFGAF

SEQ ID NO: 45 - protein, 8 aa, Artificial Sequence
  Heavy CDR3 Clone CP_01_C08
GYSSSWYL

SEQ ID NO: 46 - protein, 4 aa, Artificial Sequence
  Heavy CDR3 Clone CP_01_C11
GGAG

SEQ ID NO: 47 - protein, 9 aa, Artificial Sequence
  Heavy CDR3 Clone CP_01_F03
  misc_feature (3)..(3): Xaa can be any naturally occurring amino acid
GYXASSWSQ

SEQ ID NO: 48 - protein, 9 aa, Artificial Sequence
  Heavy CDR3 Clone CP_01_E07
DLGGSGGAF

SEQ ID NO: 49 - protein, 6 aa, Artificial Sequence
  Heavy CDR3 Clone CP_01_D03
NKQYGM

SEQ ID NO: 50 - protein, 5 aa, Artificial Sequence
  Heavy CDR3 Clone CP_01_F06
DDGAM

SEQ ID NO: 51 - protein, 6 aa, Artificial Sequence
  Heavy CDR3 Clone CP_01_F02
AGYSYA

SEQ ID NO: 52 - protein, 9 aa, Artificial Sequence
  Heavy CDR3 Clone CP_02_C03
GGRYSSNYF

SEQ ID NO: 53 - protein, 7 aa, Artificial Sequence
  Heavy CDR3 Clone CP_02_D10
VGEGSAM

SEQ ID NO: 54 - protein, 15 aa, Artificial Sequence
  Heavy CDR3 Clone CP_02_B01
VSSHYDSSGYYAGGF

SEQ ID NO: 55 - protein, 9 aa, Artificial Sequence
  Heavy CDR3 Clone CP_02_D02
GRDSSSWSP

SEQ ID NO: 56 - protein, 9 aa, Artificial Sequence
  Heavy CDR3 Clone CP_02_A02
VTTYSGLDF

SEQ ID NO: 57 - protein, 10 aa, Artificial Sequence
  Heavy CDR3 Clone CP_02_D04
KGAVVVPGAL

SEQ ID NO: 58 - protein, 9 aa, Artificial Sequence
  Heavy CDR3 Clone CP_01_E10
NSLYGGNSA

SEQ ID NO: 59 - protein, 7 aa, Artificial Sequence
  Heavy CDR3 Clone CP_01_H08
DGGGGRF

SEQ ID NO: 60 - protein, 9 aa, Artificial Sequence
  Heavy CDR3 Clone CP_01_F11
GGGALGRGM

SEQ ID NO: 61 - protein, 11 aa, Artificial Sequence
  Heavy CDR3 Clone CP_01_F09
LLRSGGQSYAF

SEQ ID NO: 62 - protein, 8 aa, Artificial Sequence
  Heavy CDR3 Clone CP_02_D05
GYSSSWSF

SEQ ID NO: 63 - protein, 8 aa, Artificial Sequence
  Heavy CDR3 Clone CP_02_A09
AGSSGWTL

SEQ ID NO: 64 - protein, 6 aa, Artificial Sequence
  Heavy CDR3 Clone CP_02_D03
DKGWGF

SEQ ID NO: 65 - protein, 11 aa, Artificial Sequence
  Heavy CDR3 Clone CP_02_C11
LGVVRGVMKGF

SEQ ID NO: 66 - protein, 9 aa, Artificial Sequence
  Heavy CDR3 Clone CP_01_H10
SSYSSSWGM

SEQ ID NO: 67 - protein, 9 aa, Artificial Sequence
  Heavy CDR3 Clone CP_02_C04
ANSWSAGGM

SEQ ID NO: 68 - protein, 9 aa, Artificial Sequence
  Heavy CDR3 Clone CP_01_G03
ERGRGYSYM

SEQ ID NO: 69 - protein, 9 aa, Artificial Sequence
  Heavy CDR3 Clone CP_01_G06
VARGIHDAF

SEQ ID NO: 70 - protein, 4 aa, Artificial Sequence
  Heavy CDR3 Clone CP_01_D06
RHGM

SEQ ID NO: 71 - protein, 8 aa, Artificial Sequence
  Heavy CDR3 Clone CP_02_B03
FDSSGYYY

SEQ ID NO: 72 - protein, 8 aa, Artificial Sequence
  Heavy CDR3 Clone CP_02_A12
DLVTTGAF

SEQ ID NO: 73 - protein, 12 aa, Artificial Sequence
  Heavy CDR3 Clone CP_01_H03
AIRVSGTPENGF

SEQ ID NO: 74 - protein, 7 aa, Artificial Sequence
  Heavy CDR3 Clone CP_01_G08
VRITHGM

SEQ ID NO: 75 - protein, 5 aa, Artificial Sequence
  Heavy CDR3 Clone CP_01_A06
GKLAF

SEQ ID NO: 76 - protein, 15 aa, Artificial Sequence
  Heavy CDR3 Clone CP_02_A04
NGDSSGYHTSPNWYF

SEQ ID NO: 77 - protein, 8 aa, Artificial Sequence
  Heavy CDR3 Clone CP_01_E08
VSTDSSSM

SEQ ID NO: 78 - protein, 8 aa, Artificial Sequence
  Heavy CDR3 Clone CP_01_A08
TSQDPGAF

SEQ ID NO: 79 - protein, 11 aa, Artificial Sequence
  Heavy CDR3 Clone CP_01_D01
AESGVYSSNGM

SEQ ID NO: 80 - protein, 8 aa, Artificial Sequence
  Heavy CDR3 Clone CP_02_A07
VDRVRSGM

SEQ ID NO: 81 - protein, 12 aa, Artificial Sequence
  Heavy CDR3 Clone CP_02_B08
IGQYCSSTSCYM

SEQ ID NO: 82 - protein, 9 aa, Artificial Sequence
  Heavy CDR3 Clone CP_01_D09
DLGGSGGAF

SEQ ID NO: 83 - protein, 7 aa, Artificial Sequence
  Heavy CDR3 Clone CP_01_G07
DSDAGYF

SEQ ID NO: 84 - protein, 9 aa, Artificial Sequence
  Heavy CDR3 Clone CP_01_A05
ASIVASGAF

SEQ ID NO: 85 - protein, 7 aa, Artificial Sequence
  Heavy CDR3 Clone CP_02_A08
AGGSNAF

SEQ ID NO: 86 - protein, 12 aa, Artificial Sequence
  Heavy CDR3 Clone CP_02_D07
VSTDSYGRQNWF

SEQ ID NO: 87 - protein, 14 aa, Artificial Sequence
  Heavy CDR3 Clone CP_01_C04
QYTSGRLAYYYHYM

SEQ ID NO: 88 - protein, 6 aa, Artificial Sequence
  Heavy CDR3 Clone CP_01_A07
GIRGAF

SEQ ID NO: 89 - protein, 8 aa, Artificial Sequence
  Heavy CDR3 Clone CP_01_H02
VGYSTTQL

SEQ ID NO: 90 - protein, 8 aa, Artificial Sequence
  Heavy CDR3 Clone CP_01_F10
MAGSYYAF

SEQ ID NO: 91 - protein, 15 aa, Artificial Sequence
  Heavy CDR3 Clone CP_02_C10
VGDYYDSSGYLDWYF

SEQ ID NO: 92 - protein, 9 aa, Artificial Sequence
  Heavy CDR3 Clone CP_02_B05
GSDTTSFVS

SEQ ID NO: 93 - protein, 8 aa, Artificial Sequence
  Heavy CDR3 Clone CP_01_G04
AGHYYYYM

SEQ ID NO: 94 - protein, 13 aa, Artificial Sequence
  Heavy CDR3 Clone CP_01_F08
VTGYPDYYDSSGF

SEQ ID NO: 95 - protein, 9 aa, Artificial Sequence
  Heavy CDR3 Clone CP_01_G05
VEGGPPYYF

SEQ ID NO: 96 - protein, 8 aa, Artificial Sequence
  Heavy CDR3 Clone CP_02_A03
NGLDNYGM

SEQ ID NO: 97 - protein, 8 aa, Artificial Sequence
  Heavy CDR3 Clone CP_01_B09
LGTTKRAF

SEQ ID NO: 98 - protein, 8 aa, Artificial Sequence
  Heavy CDR3 Clone CP_01_A10
VYVDHEGM

SEQ ID NO: 99 - protein, 6 aa, Artificial Sequence
  Heavy CDR3 Clone CP_01_H04
WSGSGF

SEQ ID NO: 100 - protein, 8 aa, Artificial Sequence
  Heavy CDR3 Clone CP_02_B04
DFGWGGAF

SEQ ID NO: 101 - protein, 7 aa, Artificial Sequence
  Heavy CDR3 Clone CP_02_A05
VVGGTQH

SEQ ID NO: 102 - protein, 14 aa, Artificial Sequence
  Heavy CDR3 Clone CP_01_F07
NWLLYYGDPQQNAF

SEQ ID NO: 103 - protein, 13 aa, Artificial Sequence
  Heavy CDR3 Clone CP_01_H01
LYFDWFADSQNAF

SEQ ID NO: 104 - protein, 13 aa, Artificial Sequence
  Heavy CDR3 Clone CP_01_G10
VGYQPLLYADYYF

SEQ ID NO: 105 - protein, 5 aa, Artificial Sequence
  Heavy CDR3 Clone CP_01_G11
GAMGL

SEQ ID NO: 106 - protein, 10 aa, Artificial Sequence
  Heavy CDR3 Clone CP_01_G01
VYYLSGVHAF

SEQ ID NO: 107 - protein, 7 aa, Artificial Sequence
  Heavy CDR3 Clone CP_01_A12
TERWLQF

SEQ ID NO: 108 - protein, 6 aa, Artificial Sequence
  Heavy CDR3 Clone CP_01_H05
NGDYAF

SEQ ID NO: 109 - protein, 10 aa, Artificial Sequence
  Heavy CDR3 Clone CP_02_B07
ASRYSGSYHF

SEQ ID NO: 110 - protein, 7 aa, Artificial Sequence
  Heavy CDR3 Clone CP_01_G09
HGSQGGF

SEQ ID NO: 111 - protein, 7 aa, Artificial Sequence
  Heavy CDR3 Clone CP_02_C02
VGYMGGM

SEQ ID NO: 112 - protein, 10 aa, Artificial Sequence
  Heavy CDR3 Clone CP_01_D05
NTPGIAAAGP

SEQ ID NO: 113 - protein, 9 aa, Artificial Sequence
  Heavy CDR3 Clone CP_01_D08
VGTTTVTSF

SEQ ID NO: 114 - protein, 9 aa, Artificial Sequence
  Heavy CDR3 Clone CP_02_A11
VGGPLNDAF

SEQ ID NO: 115 - protein, 7 aa, Artificial Sequence
  Heavy CDR3 Clone CP_02_D08
HSSGGAF

SEQ ID NO: 116 - protein, 7 aa, Artificial Sequence
  Light CDR3 Clone CP_01_E09
QQYDNLP

SEQ ID NO: 117 - protein, 7 aa, Artificial Sequence
  Light CDR3 Clone CP_01_D12
QQFNAYP

SEQ ID NO: 118 - protein, 7 aa, Artificial Sequence
  Light CDR3 Clone CP_01_D10
QQYNSYP

SEQ ID NO: 119 - protein, 7 aa, Artificial Sequence
  Light CDR3 Clone CP_01_C08
QQYDNLP

SEQ ID NO: 120 - protein, 7 aa, Artificial Sequence
  Light CDR3 Clone CP_01_C11
QSHDSSN

SEQ ID NO: 121 - protein, 7 aa, Artificial Sequence
  Light CDR3 Clone CP_01_F03
QQYDNLP

SEQ ID NO: 122 - protein, 6 aa, Artificial Sequence
  Light CDR3 Clone CP_01_E07
MQSIQL

SEQ ID NO: 123 - protein, 7 aa, Artificial Sequence
  Light CDR3 Clone CP_01_D03
QQYHRWP

SEQ ID NO: 124 - protein, 7 aa, Artificial Sequence
  Light CDR3 Clone CP_01_F06
QQYYDSP

SEQ ID NO: 125 - protein, 7 aa, Artificial Sequence
  Light CDR3 Clone CP_01_F02
LQHNSYP

SEQ ID NO: 126 - protein, 6 aa, Artificial Sequence
  Light CDR3 Clone CP_02_C03
QQYFGT

SEQ ID NO: 127 - protein, 7 aa, Artificial Sequence
  Light CDR3 Clone CP_02_D10
QQYNDWP

SEQ ID NO: 128 - protein, 8 aa, Artificial Sequence
  Light CDR3 Clone CP_02_B01
QSFDTNSL

SEQ ID NO: 129 - protein, 7 aa, Artificial Sequence
  Light CDR3 Clone CP_02_D02
QQYDNLP

SEQ ID NO: 130 - protein, 8 aa, Artificial Sequence
  Light CDR3 Clone CP_02_A02
SSYTSSST

SEQ ID NO: 131 - protein, 7 aa, Artificial Sequence
  Light CDR3 Clone CP_02_D04
QQYNSYP

SEQ ID NO: 132 - protein, 7 aa, Artificial Sequence
  Light CDR3 Clone CP_01_E10
QQTFTTP

SEQ ID NO: 133 - protein, 7 aa, Artificial Sequence
  Light CDR3 Clone CP_01_H08
QQYDNLP

SEQ ID NO: 134 - protein, 7 aa, Artificial Sequence
  Light CDR3 Clone CP_01_F11
QSYDTNN

SEQ ID NO: 135 - protein, 7 aa, Artificial Sequence
  Light CDR3 Clone CP_01_F09
QQSYSTP

SEQ ID NO: 136 - protein, 7 aa, Artificial Sequence
  Light CDR3 Clone CP_02_D05
QQYDNLP

SEQ ID NO: 137 - protein, 7 aa, Artificial Sequence
  Light CDR3 Clone CP_02_A09
QQYDNLP

SEQ ID NO: 138 - protein, 7 aa, Artificial Sequence
  Light CDR3 Clone CP_02_D03
QQYDNLP

SEQ ID NO: 139 - protein, 7 aa, Artificial Sequence
  Light CDR3 Clone CP_02_C11
QSYDSSN

SEQ ID NO: 140 - protein, 7 aa, Artificial Sequence
  Light CDR3 Clone CP_01_H10
QQYDNLP

SEQ ID NO: 141 - protein, 7 aa, Artificial Sequence
  Light CDR3 Clone CP_02_C04
QQYDDLP

SEQ ID NO: 142 - protein, 7 aa, Artificial Sequence
  Light CDR3 Clone CP_01_G03
MQRIEFP

SEQ ID NO: 143 - protein, 8 aa, Artificial Sequence
  Light CDR3 Clone CP_01_G06
QSYDNTRH

SEQ ID NO: 144 - protein, 7 aa, Artificial Sequence
  Light CDR3 Clone CP_01_D06
QSYDSSN

SEQ ID NO: 145 - protein, 7 aa, Artificial Sequence
  Light CDR3 Clone CP_02_B03
QSYDSSN

SEQ ID NO: 146 - protein, 7 aa, Artificial Sequence
  Light CDR3 Clone CP_02_A12
QQHNDWP

SEQ ID NO: 147 - protein, 8 aa, Artificial Sequence
  Light CDR3 Clone CP_01_H03
QSYHSSNL

SEQ ID NO: 148 - protein, 7 aa, Artificial Sequence
  Light CDR3 Clone CP_01_G08
MQATHFP

SEQ ID NO: 149 - protein, 7 aa, Artificial Sequence
  Light CDR3 Clone CP_01_A06
QQYYSTP

SEQ ID NO: 150 - protein, 8 aa, Artificial Sequence
  Light CDR3 Clone CP_02_A04
QSYDDSNY

SEQ ID NO: 151 - protein, 9 aa, Artificial Sequence
  Light CDR3 Clone CP_01_E08
SSYAGSNTL

SEQ ID NO: 152 - protein, 7 aa, Artificial Sequence
  Light CDR3 Clone CP_01_A08
QQANSFP

SEQ ID NO: 153 - protein, 7 aa, Artificial Sequence
  Light CDR3 Clone CP_01_D01
QSYDSSI

SEQ ID NO: 154 - protein, 7 aa, Artificial Sequence
  Light CDR3 Clone CP_02_A07
QSYDSIH

SEQ ID NO: 155 - protein, 8 aa, Artificial Sequence
  Light CDR3 Clone CP_02_B08
QSYDSSTH

SEQ ID NO: 156 - protein, 7 aa, Artificial Sequence
  Light CDR3 Clone CP_01_D09
QQYDNLP

SEQ ID NO: 157 - protein, 8 aa, Artificial Sequence
  Light CDR3 Clone CP_01_G07
QSFTSSTL

SEQ ID NO: 158 - protein, 7 aa, Artificial Sequence
  Light CDR3 Clone CP_01_A05
QQYNKWP

SEQ ID NO: 159 - protein, 8 aa, Artificial Sequence
  Light CDR3 Clone CP_02_A08
QSYDDSNY

SEQ ID NO: 160 - protein, 8 aa, Artificial Sequence
  Light CDR3 Clone CP_02_D07
QSYDSSNH

SEQ ID NO: 161 - protein, 7 aa, Artificial Sequence
  Light CDR3 Clone CP_01_C04
QQSYSTP

SEQ ID NO: 162 - protein, 10 aa, Artificial Sequence
  Light CDR3 Clone CP_01_A07
NSRDSSGNPN

SEQ ID NO: 163 - protein, 8 aa, Artificial Sequence
  Light CDR3 Clone CP_01_H02
QSYDSSNL

SEQ ID NO: 164 - protein, 8 aa, Artificial Sequence
  Light CDR3 Clone CP_01_F10
QSYDSSNH

SEQ ID NO: 165 - protein, 9 aa, Artificial Sequence
  Light CDR3 Clone CP_02_C10
AVWDDRLNG

SEQ ID NO: 166 - protein, 7 aa, Artificial Sequence
  Light CDR3 Clone CP_02_B05
QQYDSYS

SEQ ID NO: 167 - protein, 9 aa, Artificial Sequence
  Light CDR3 Clone CP_01_G04
QSADSSGTN

SEQ ID NO: 168 - protein, 8 aa, Artificial Sequence
  Light CDR3 Clone CP_01_F08
QSYDSSNH

SEQ ID NO: 169 - protein, 8 aa, Artificial Sequence
  Light CDR3 Clone CP_01_G05
QSYDTRNQ

SEQ ID NO: 170 - protein, 7 aa, Artificial Sequence
  Light CDR3 Clone CP_02_A03
QQYYSTP

SEQ ID NO: 171 - protein, 6 aa, Artificial Sequence
  Light CDR3 Clone CP_01_B09
QQSYST

SEQ ID NO: 172 - protein, 8 aa, Artificial Sequence
  Light CDR3 Clone CP_01_A10
AAWDDSLF

SEQ ID NO: 173 - protein, 7 aa, Artificial Sequence
  Light CDR3 Clone CP_01_H04
MQRIEFP

SEQ ID NO: 174 - protein, 7 aa, Artificial Sequence
  Light CDR3 Clone CP_02_B04
QQYYNTP

SEQ ID NO: 175 - protein, 7 aa, Artificial Sequence
  Light CDR3 Clone CP_02_A05
QSYDSSI

SEQ ID NO: 176 - protein, 8 aa, Artificial Sequence
  Light CDR3 Clone CP_01_F07
QSYDSTNL

SEQ ID NO: 177 - protein, 7 aa, Artificial Sequence
  Light CDR3 Clone CP_01_H01
QQSYSTP

SEQ ID NO: 178 - protein, 9 aa, Artificial Sequence
  Light CDR3 Clone CP_01_G10
NSRDSSGNH

SEQ ID NO: 179 - protein, 7 aa, Artificial Sequence
  Light CDR3 Clone CP_01_G11
QQSYSTP

SEQ ID NO: 180 - protein, 9 aa, Artificial Sequence
  Light CDR3 Clone CP_01_G01
  misc_feature (9)..(9): Xaa can be any naturally occurring amino acid
DSRDTRVNX

SEQ ID NO: 181 - protein, 6 aa, Artificial Sequence
  Light CDR3 Clone CP_01_A12
QQYDNL

SEQ ID NO: 182 - protein, 7 aa, Artificial Sequence
  Light CDR3 Clone CP_01_H05
MQALQTP

SEQ ID NO: 183 - protein, 7 aa, Artificial Sequence
  Light CDR3 Clone CP_02_B07
QSYDSSN

SEQ ID NO: 184 - protein, 8 aa, Artificial Sequence
  Light CDR3 Clone CP_01_G09
SSYTSSST

SEQ ID NO: 185 - protein, 8 aa, Artificial Sequence
  Light CDR3 Clone CP_02_C02
MQALQTPP

SEQ ID NO: 186 - protein, 8 aa, Artificial Sequence
  Light CDR3 Clone CP_01_D05
QSYDSTNH

SEQ ID NO: 187 - protein, 8 aa, Artificial Sequence
  Light CDR3 Clone CP_01_D08
QSYDSANL

SEQ ID NO: 188 - protein, 8 aa, Artificial Sequence
  Light CDR3 Clone CP_02_A11
QSFDENIS

SEQ ID NO: 189 - protein, 7 aa, Artificial Sequence
  Light CDR3 Clone CP_02_D08
HQSATSP

SEQ ID NO: 190 - protein, 7 aa, Artificial Sequence
  Heavy CDR3 Clone CP_03_E05
TRSSGAF

SEQ ID NO: 191 - protein, 8 aa, Artificial Sequence
  Heavy CDR3 Clone CP_03_D05
PRGRGSAF

SEQ ID NO: 192 - protein, 6 aa, Artificial Sequence
  Heavy CDR3 Clone CP_03_H06
ARVGGM

SEQ ID NO: 193 - protein, 5 aa, Artificial Sequence
  Heavy CDR3 Clone CP_03_C12
DTGPI

SEQ ID NO: 194 - protein, 11 aa, Artificial Sequence
  Heavy CDR3 Clone CP_03_G02
GVWNSGSYLGF

SEQ ID NO: 195 - protein, 10 aa, Artificial Sequence
  Heavy CDR3 Clone CP_03_D04
GGFTVPGGAF

SEQ ID NO: 196 - protein, 7 aa, Artificial Sequence
  Heavy CDR3 Clone CP_03_F10
FGERYAF

SEQ ID NO: 197 - protein, 12 aa, Artificial Sequence
  Heavy CDR3 Clone CP_03_G09
DQWLANYYYYGM

SEQ ID NO: 198 - protein, 11 aa, Artificial Sequence
  Heavy CDR3 Clone CP_03_F09
NRGGSYKSVGM

SEQ ID NO: 199 - protein, 7 aa, Artificial Sequence
  Heavy CDR3 Clone CP_03_D09
VSSYYGM

SEQ ID NO: 200 - protein, 7 aa, Artificial Sequence
  Heavy CDR3 Clone CP_03_F02
APASSAH

SEQ ID NO: 201 - protein, 8 aa, Artificial Sequence
  Heavy CDR3 Clone CP_02_E03
QRGYYYGM

SEQ ID NO: 202 - protein, 8 aa, Artificial Sequence
  Heavy CDR3 Clone CP_03_H07
SSVAAGAF

SEQ ID NO: 203 - protein, 8 aa, Artificial Sequence
  Heavy CDR3 Clone CP_03_C02
LSGRGLGF

SEQ ID NO: 204 - protein, 4 aa, Artificial Sequence
  Heavy CDR3 Clone CP_03_E09
DHYF

SEQ ID NO: 205 - protein, 8 aa, Artificial Sequence
  Heavy CDR3 Clone CP_03_D08
SGRRVTAI

SEQ ID NO: 206 - protein, 8 aa, Artificial Sequence
  Heavy CDR3 Clone CP_03_E11
MGRYSSSW

SEQ ID NO: 207 - protein, 8 aa, Artificial Sequence
  Heavy CDR3 Clone CP_03_B05
HSRFGPAF

SEQ ID NO: 208 - protein, 5 aa, Artificial Sequence
  Heavy CDR3 Clone CP_03_H02
DREAF

SEQ ID NO: 209 - protein, 13 aa, Artificial Sequence
  Heavy CDR3 Clone CP_03_D02
LRGRYSYGYSDAF

SEQ ID NO: 210 - protein, 9 aa, Artificial Sequence
  Heavy CDR3 Clone CP_03_E01
LLNAVTYAF

SEQ ID NO: 211 - protein, 7 aa, Artificial Sequence
  Heavy CDR3 Clone CP_03_C11
IGVIGGF

SEQ ID NO: 212 - protein, 10 aa, Artificial Sequence
  Heavy CDR3 Clone CP_03_B12
IEYSSSSPYF

SEQ ID NO: 213 - protein, 13 aa, Artificial Sequence
  Heavy CDR3 Clone CP_03_E03
DLLPTTVTTTGAF

SEQ ID NO: 214 - protein, 6 aa, Artificial Sequence
  Heavy CDR3 Clone CP_03_F01
DSGSYS

SEQ ID NO: 215 - protein, 12 aa, Artificial Sequence
  Heavy CDR3 Clone CP_03_C01
ASYPYYYYYYGM

SEQ ID NO: 216 - protein, 5 aa, Artificial Sequence
  Heavy CDR3 Clone CP_03_G07
ALGHF

SEQ ID NO: 217 - protein, 7 aa, Artificial Sequence
  Heavy CDR3 Clone CP_03_E02
FTTGSAL

SEQ ID NO: 218 - protein, 5 aa, Artificial Sequence
  Heavy CDR3 Clone CP_03_C07
DASGY

SEQ ID NO: 219 - protein, 11 aa, Artificial Sequence
  Heavy CDR3 Clone CP_03_H04
DLGTYYYGSGD

SEQ ID NO: 220 - protein, 8 aa, Artificial Sequence
  Heavy CDR3 Clone CP_03_E06
VGELLGAF

SEQ ID NO: 221 - protein, 8 aa, Artificial Sequence
  Light CDR3 Clone CP_03_E05
HSYDSNNH

SEQ ID NO: 222 - protein, 9 aa, Artificial Sequence
  Heavy CDR3 Clone CP_03_G11
HSGVGGLAF

SEQ ID NO: 223 - protein, 10 aa, Artificial Sequence
  Heavy CDR3 Clone CP_03_G01
GGSIAAALAF

SEQ ID NO: 224 - protein, 8 aa, Artificial Sequence
  Heavy CDR3 Clone CP_03_H01
VEYSRNGM

SEQ ID NO: 225 - protein, 4 aa, Artificial Sequence
  Heavy CDR3 Clone CP_03_F11
GRYN

SEQ ID NO: 226 - protein, 7 aa, Artificial Sequence
  Heavy CDR3 Clone CP_03_C06
LDYYYGM

SEQ ID NO: 227 - protein, 7 aa, Artificial Sequence
  Heavy CDR3 Clone CP_03_D03
GGLSSAF

SEQ ID NO: 228 - protein, 5 aa, Artificial Sequence
  Heavy CDR3 Clone CP_03_G05
YGGGL

SEQ ID NO: 229 - protein, 7 aa, Artificial Sequence
  Heavy CDR3 Clone CP_03_G12
PDHLTVF

SEQ ID NO: 230 - protein, 6 aa, Artificial Sequence
  Heavy CDR3 Clone CP_03_C10
VGYYGM

SEQ ID NO: 231 - protein, 7 aa, Artificial Sequence
  Heavy CDR3 Clone CP_03_F04
YEGYAGF

SEQ ID NO: 232 - protein, 8 aa, Artificial Sequence
  Light CDR3 Clone CP_03_D05
QAWDTNLG

SEQ ID NO: 233 - protein, 9 aa, Artificial Sequence
  Light CDR3 Clone CP_03_H06
QSFDADNLH

SEQ ID NO: 234 - protein, 7 aa, Artificial Sequence
  Light CDR3 Clone CP_03_C12
MQGIQLP

SEQ ID NO: 235 - protein, 7 aa, Artificial Sequence
  Light CDR3 Clone CP_03_G02
QQSYSTP

SEQ ID NO: 236 - protein, 7 aa, Artificial Sequence
  Light CDR3 Clone CP_03_D04
QSYDASN

SEQ ID NO: 237 - protein, 7 aa, Artificial Sequence
  Light CDR3 Clone CP_03_F10
SAYTGSN

SEQ ID NO: 238 - protein, 7 aa, Artificial Sequence
  Light CDR3 Clone CP_03_G09
QVWDSNS

SEQ ID NO: 239 - protein, 7 aa, Artificial Sequence
  Light CDR3 Clone CP_03_F09
QSYDEVS

SEQ ID NO: 240 - protein, 8 aa, Artificial Sequence
  Light CDR3 Clone CP_03_D09
QSYNSSNH

SEQ ID NO: 241 - protein, 7 aa, Artificial Sequence
  Light CDR3 Clone CP_03_F02
QSYDSSH

SEQ ID NO: 242 - protein, 7 aa, Artificial Sequence
  Light CDR3 Clone CP_02_E03
QQSRSTP

SEQ ID NO: 243 - protein, 8 aa, Artificial Sequence
  Light CDR3 Clone CP_03_H07
SSYTSSST

SEQ ID NO: 244 - protein, 9 aa, Artificial Sequence
  Light CDR3 Clone CP_03_C02
SSYAGSSNL

SEQ ID NO: 245 - protein, 8 aa, Artificial Sequence
  Light CDR3 Clone CP_03_E09
NSYTRSST

SEQ ID NO: 246 - protein, 7 aa, Artificial Sequence
  Light CDR3 Clone CP_03_D08
QSYDDTN

SEQ ID NO: 247 - protein, 8 aa, Artificial Sequence
  Light CDR3 Clone CP_03_E11
QAWDTNIG

SEQ ID NO: 248 - protein, 8 aa, Artificial Sequence
  Light CDR3 Clone CP_03_B05
SSYAGSNN

SEQ ID NO: 249 - protein, 8 aa, Artificial Sequence
  Light CDR3 Clone CP_03_H02
QAWDTNIG

SEQ ID NO: 250 - protein, 8 aa, Artificial Sequence
  Light CDR3 Clone CP_03_D02
SSYAGSST

SEQ ID NO: 251 - protein, 8 aa, Artificial Sequence
  Light CDR3 Clone CP_03_E01
NSRDSSGF

SEQ ID NO: 252 - protein, 7 aa, Artificial Sequence
  Light CDR3 Clone CP_03_C11
SSYTSSS

SEQ ID NO: 253 - protein, 8 aa, Artificial Sequence
  Light CDR3 Clone CP_03_B12
QSYDSNNR

SEQ ID NO: 254 - protein, 9 aa, Artificial Sequence
  Light CDR3 Clone CP_03_E03
SSRDSSGNH

SEQ ID NO: 255 - protein, 8 aa, Artificial Sequence
  Light CDR3 Clone CP_03_F01
QAWDTNIG

SEQ ID NO: 256 - protein, 9 aa, Artificial Sequence
  Light CDR3 Clone CP_03_C01
QVWDSSTAN

SEQ ID NO: 257 - protein, 9 aa, Artificial Sequence
  Light CDR3 Clone CP_03_G07
QSYDSSNHH

SEQ ID NO: 258 - protein, 8 aa, Artificial Sequence
  Light CDR3 Clone CP_03_E02
SSYAGNSN

SEQ ID NO: 259 - protein, 8 aa, Artificial Sequence
  Light CDR3 Clone CP_03_C07
QAWDTNIG

SEQ ID NO: 260 - protein, 10 aa, Artificial Sequence
  Light CDR3 Clone CP_03_H04
GTWDSSLSAG

SEQ ID NO: 261 - protein, 9 aa, Artificial Sequence
  Light CDR3 Clone CP_03_E06
SSLDSNDNH

SEQ ID NO: 262 - protein, 9 aa, Artificial Sequence
  Light CDR3 Clone CP_03_H03
AAWDDSLNG

SEQ ID NO: 263 - protein, 8 aa, Artificial Sequence
  Light CDR3 Clone CP_03_G11
SSYAGSST

SEQ ID NO: 264 - protein, 7 aa, Artificial Sequence
  Light CDR3 Clone CP_03_G01
HQYDVYP

SEQ ID NO: 265 - protein, 9 aa, Artificial Sequence
  Light CDR3 Clone CP_03_H01
NSRDSSGNH

SEQ ID NO: 266 - protein, 7 aa, Artificial Sequence
  Light CDR3 Clone CP_03_F11
QSYDSSN

SEQ ID NO: 267 - protein, 7 aa, Artificial Sequence
  Light CDR3 Clone CP_03_C06
QSYDSSN

SEQ ID NO: 268 - protein, 7 aa, Artificial Sequence
  Light CDR3 Clone CP_03_D03
QSYDSSN

SEQ ID NO: 269 - protein, 8 aa, Artificial Sequence
  Light CDR3 Clone CP_03_G05
SSYAGSYT

SEQ ID NO: 270 - protein, 7 aa, Artificial Sequence
  Light CDR3 Clone CP_03_G12
SSYTPSS

SEQ ID NO: 271 - protein, 8 aa, Artificial Sequence
  Light CDR3 Clone CP_03_C10
QAWDTNIG

SEQ ID NO: 272 - protein, 8 aa, Artificial Sequence
  Light CDR3 Clone CP_03_F04
QAWDTNIG

(19) United States
(12) Patent Application Publication
(10) Pub. No.: US 2017/0066827 A1
(43) Pub. Date: Mar. 9, 2017

(54) CHIMERIC ANTIGEN RECEPTOR

(71) Applicant: UCL BUSINESS PLC, London (GB)

(72) Inventors: Martin Pulé, London (GB); Paul Maciocia, London (GB)

(21) Appl. No.: 15/123,287

(22) PCT Filed: Mar. 5, 2015
(86) PCT No.: PCT/GB2015/050643

§ 371 Date: Sep. 2, 2016

(30) Foreign Application Priority Data
Mar. 5, 2014 (GB) 1403905.1
Sep. 25, 2014 (GB) 1416908.0

(51) Int. Cl. C07K 16/28; G01N 33/574; C07K 14/725

(52) U.S. Cl. CPC C07K 16/2809; C07K 14/7051; G01N 33/57426; C07K 2317/565; C07K 2317/56; C07K 2317/622; G01N 2333/7051

(57) ABSTRACT

The present disclosure relates to a chimeric antigen receptor (CAR) which comprises an antigen-binding domain which selectively binds TCR beta constant region 1 (TRBC1) or TRBC2; cells; such a T cells comprising such a CAR; and the use of such cells for the treatment of a T-cell lymphoma or leukaemia in a subject.

### FIELD OF THE INVENTION

The present invention relates to cells and agents useful in the treatment of T-cell lymphoma or leukaemia.

### BACKGROUND TO THE INVENTION

Lymphoid malignancies can largely be divided into those which are derived from either T-cells or B-cells. T-cell malignancies are a clinically and biologically heterogeneous group of disorders, together comprising 10-20% of non-Hodgkin's lymphomas and 20% of acute leukaemias. The most commonly identified histological subtypes are peripheral T-cell lymphoma, not otherwise specified (PTCL-NOS); angio-immunoblastic T-cell lymphoma (AITL) and anaplastic large cell lymphoma (ALCL). Of all acute Lymphoblastic Leukaemias (ALL), some 20% are of a T-cell phenotype.

These conditions typically behave aggressively, compared for instance with B-cell malignancies, with estimated 5-year survival of only 30%. In the case of T-cell lymphoma, they are associated with a high proportion of patients presenting with disseminated disease, unfavourable International Prognostic Indicator (IPI) score and prevalence of extra-nodal disease. Chemotherapy alone is not usually effective and less than 30% of patients are cured with current treatments.

Further, unlike in B-cell malignancies, where immunotherapies such as the anti-CD20 monoclonal antibody rituximab have dramatically improved outcomes, there is currently no equivalently effective, minimally toxic immunotherapeutic available for the treatment of T-cell malignancies. An important difficulty in the development of immunotherapy for T-cell disorders is the considerable overlap in marker expression of clonal and normal T-cells, with no single antigen clearly able to identify clonal (malignant) cells.

The same problem exists when targeting a pan-B-cell antigen to treat a B-cell malignancy. However, in this case, the concomitant depletion of the B-cell compartment results in relatively minor immunosuppression which is readily tolerated by most patients. Further, in therapies which result in particularly long-term depletion of the normal B-compartment, its loss can be largely abrogated by administration of pooled immunoglobulin. The situation is completely different when targeting T-cell malignancies. Here, concomitant depletion of the T-cell compartment leads to severe immunosuppression and severe toxicity. Further, there is no satisfactory way to mitigate loss of the T-cell compartment.

The toxicity is in part illustrated by the clinical effects of the therapeutic monoclonal antibody Alemtuzumab. This agent lyses cells which express CD52 and has some efficacy in T-cell malignancies. The utility of this agent is greatly limited by a profound cellular immunodeficiency, largely due to T-cell depletion, with markedly elevated risk of infection.

There is thus a need for a new method for targeted treatment of T-cell malignancies which is not associated with the above disadvantages.

### DESCRIPTION OF THE FIGURES

FIG. 1: A diagram of the αβ T-cell Receptor/CD3 Complex. The T-cell receptor is formed from 6 different protein chains which must assemble in the endoplasmic reticulum to be expressed on the cell surface. The four proteins of the CD3 complex (CD3ζ, CD3γ, CD3ε and CD3δ) sheath the T-cell Receptor (TCR). This TCR imbues the complex with specificity of a particular antigen and is composed of two chains: TCRα and TCRβ. Each TCR chain has a variable component distal to the membrane and a constant component proximal to the membrane. Nearly all T-cell lymphomas and many T-cell leukaemias express the TCR/CD3 complex.

FIG. 2: The segregation of T-cell Receptor n-constant region (TRBC)-1 and TRBC2 during T-cell receptor rearrangement. Each TCR beta chain is formed from genomic recombination of a particular beta variable (V), diversity (D), joining (J) and constant (TRBC) regions. The human genome contains two very similar and functionally equivalent TRBC loci known as TRBC1 and TRBC2. During TCR gene re-arrangement, a J-region recombines with either TRBC1 or TRBC2. This rearrangement is permanent. T-cells express many copies of a single TCR on their surface, hence each T-cell will express a TCR whose 13-chain constant region is coded for by either TRBC1 or TRBC2.

FIG. 3: Alignment of human TRBC1 and TRBC2 at the amino acid level. The TCRβ constant chain coded for by TRBC1 and TRBC2 differ by only 4 amino acid differences: K/N at position 3 of the TRBC; N/K at position 4 of the TRBC; F/Y at position 36 of the TRBC; V/E at position 135 of the TRBC;

FIG. 4: Definitive demonstration that the JOVI-1 antibody binds to TRBC1 but not TRBC2. Genetic engineering of cells was used to definitively demonstrate that the JOVI-1 monoclonal antibody recognizes TRBC1 variant of the TCRβ constant chain. A tri-cistronic retroviral cassette was generated. This coded for both the TCRα and TCRβ chains of a human TCR which recognizes the minor histocompatibility antigen HA1, along with truncated human CD34 as a convenient marker gene. The HA1 TCR is natively TRBC2. A second retroviral cassette was generated which was identical to the first except the 4 residues in the TCR P constant region which differentiate TRBC1 from TRBC2 were changed to those coded by TRBC1. Jurkat T-cells which have both their TCRα and TCRβ chains knocked-out were transduced with either vector. These cells were stained with either a pan-TCR/CD3 antibody or the monoclonal JOVI-1 conjugated along with antibodies against CD34 and analysed in a flow cytometer. The upper row demonstrates staining with a pan-TCR/CD3 antibody v CD34 (marker of transduction), the lower row demonstrates staining with

JOVI-1 vs CD34. Transduced cells demonstrate similar TCR/CD3 staining but only TRBC1 +ve cells stain with JOVI-1. Hence, JOVI-1 is specific to TRBC1 and further it is possible to use an antibody to distinguish TRBC1 and 2 TCRs.

FIG. 5: The JOVI-1 mAb differentiates TRBC1 from TRBC2 by recognizing residues 3 and 4 of the TRBC. To precisely determine how JOVI-1 discriminates TRBC1 from TRBC2, the HA1 TCR TRBC2 construct detailed above was mutated to make two TRBC1/2 hybrids. An additional variant was generated so that only residues 3 and 4 of the TCRβ constant chain were changed from those of TRBC2 to those found in TRBC1. A further variant was made where only residue 36 was changed from that found in TRBC2 to TRBC1. TCR knock-out Jurkat T-cells were transduced with these new constructs. The original TRBC2 and TRBC1 transduced Jurkats described in FIG. 4 were used as controls. The Jurkat T-cells were stained with JOVI-1 and analysed with a flow cytometer. Staining of JOVI-1 is overlaid over that of non-transduced TCR knock-out Jurkat T-cells. JOVI-1 stained Jurkats expressing the TRBC1 TCR but not the TRBC2 TCR. JOVI-1 stained TRBC1/2 hybrid where only TRBC residues 3 and 4 were those of TRBC1. JOVI-1 did not stain Jurkat T-cells where only TRBC residue 36 was that of TRBC1.

FIG. 6: Example of normal donor T-cell expression of TRBC1. Normal donor peripheral blood mononuclear cells were stained with antibodies against CD3, CD4, CD8 and JOVI-1 and analysed by flow cytometry. CD4+ and CD8+ T-cell populations are shown on the upper panel. Each of this population is gated and forward scatter vs JOVI-1 staining are shown on the Y and X-axes respectively. These data show that both CD4+ and CD8+ compartments contain cells which are TRBC1 +ve and −ve. This is representative data from one donor.

FIG. 7: TRBC1+ T-cells in several normal donors. Ten normal donors were bled and peripheral blood mononuclear cells were stained as described in FIG. 4 above. The aggregate data of the proportion of TRBC1 T-cells in both CD4 and CD8 compartments is shown in a bar graph along with median and range. All donors had TRBC1+ and TRBC1− compartments. Median % of TRBC1+ cells=36%.

FIG. 8: T-cell malignancy derived cell lines stained with JOVI-1. Several cell lines have been derived from T-cell malignancies. Many of these cell lines still express the TCR. We selected Jurkats (A T-cell leukaemia cell line), HPB-ALL (another T-cell leukaemia cell line) and HD-Mar-2 (a T-cell lymphoma cell line) for study. By staining these cell lines with a pan-TCR/CD3 antibody, we were able to demonstrate that all three express TCR (left panels, staining overlaid over isotype control staining). Next, by staining with JOVI-1 we were able to determine that these T-cell lines are either TRBC1 negative or positive. Only Jurkats cells (TRBC1+) and not HPB-ALL or HD-Mar-2 (TRBC2+) cells stain with JOVI-1, supporting exclusive expression of either TRBC1 or 2.

FIG. 9: Selective Killing of TRBC1 T-cells with JOVI-1 mAb. Wild-type Jurkat T-cells (CD34−, TRBC1+) were mixed with TCRαβ knock-out Jurkat T-cells transduced with TRBC2 co-expressed with the CD34 marker gene (CD34+TRBC2+). These cells were incubated with JOVI-1 alone or incubated with JOVI-1 and complement for 1 hour. Cells were washed and stained for CD34, Annexin V and 7-AAD. Cells were analysed by flow-cytometry. Shown below is CD34 expression in the live population as defined by Annexin-V negative and 71AAD dim population. (a) JOVI-1 alone; (b) JOVI-1 with complement. Selective killing of TRBC1 T-cells (CD34−) is observed.

FIG. 10: Polyclonal Epstein Barr Virus (EBV) specific T-cells can be split into two approximately equal TRBC1/2 populations. Using well established methods, the inventors selectively expanded EBV specific T-cells from the peripheral blood of a normal donor. The subsequent line had a high degree of selectively against autologous EBV infected B-cells (auto LCLs), and no activity against allogeneic EBV infected T-cells (allo LCLs), and no non-specific NK activity (as measured by testing against K562 cells). Such a line is representative of the donor's EBV immunity. (b) When stained with JOVI-1, these T-cells typed approximately equally for TRBC1 and TRBC2.

FIG. 11: Annotated sequence of JOVI-1 VH and VL. The hypervariable regions are underlined and in bold.

FIG. 12: Demonstration that a peripheral T-cell lymphoma is TRBC restricted, but normal circulating T-cells are not. Peripheral blood T-cells from a patient with circulating T-cell lymphoma cells were drawn. Peripheral mononuclear cells were isolated and stained with a panel of antibodies including CD5, TCR and JOVI-1. Normal and malignant T-cells could be differentiated on flow by CD5 expression intensity. CD5 bright (normal T-cells) had approximately equal TRBC1 and 2 populations. The CD5 intermediate and dim populations (the tumour) were all TRBC2 positive. If this patient had a TRBC2 directed therapy, the lymphoma would be eradicated and approximately half of their T-cells would be spared.

FIG. 13: Demonstration that the VH and VL derived from JOVI-1 were correct and that they can fold as a single-chain variable fragment. Original hybridoma supernatant, recombinant JOVI-1 antibody and scFv-Fc generated from transfected 293T cells were used to stain a number of cell lines: Jurkat TCR knock-outs, wild-type Jurkats, Jurkat TCR knock-out transduced with a TRBC1 TCR in a vector co-expressing eBFP2; Jurkat TCR knock-out transduced with a TRBD2 TCR in a vector co-expressing eBFP2. Staining was analysed by flow cytometry. Both the recombinant antibody and the scFv bound cells expressing TRBC2.

FIG. 14: JOVI-1 based CARs in different formats. CARs typically comprise of a binding domain, a spacer, a transmembrane domain and an intracellular signalling domain. In this study CARs were generated which comprise of the JOVI-1 scFv; a spacer derived from either the CD8 stalk, the hinge-CH2-CH3 domain of human IgG1 with mutations which remove FcR binding; or a spacer derived from human IgG1.

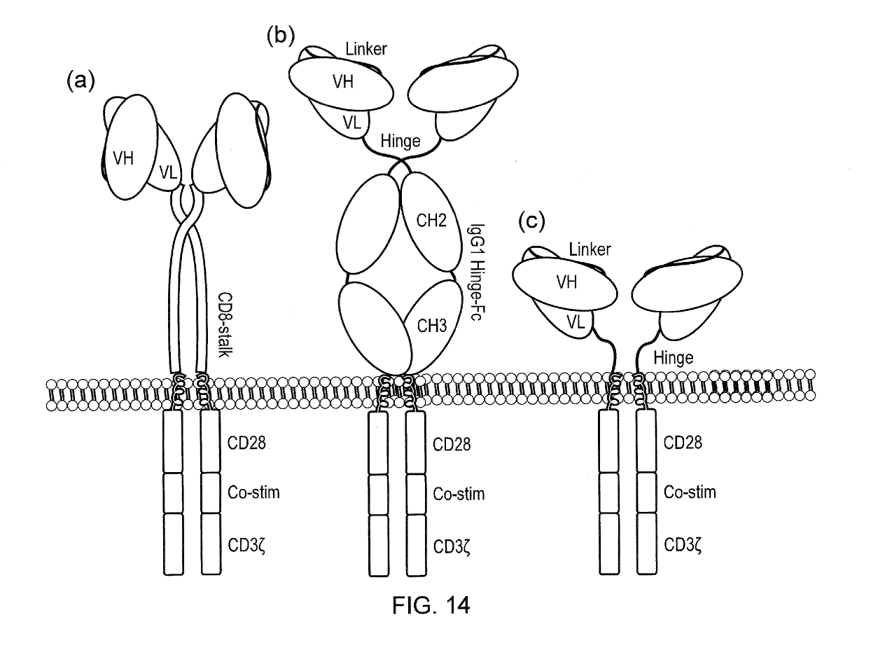

FIG. 15: Function of JOVI-1 based CAR. Normal donor peripheral blood T-cells were transduced with the different CARs described above. T-cells were also transduced with a CD19-specific CAR as a control. These T-cells were then challenged with target cells: Jurkats—TCR knock-out and Jurkats wild-type and Raji cells (a CD19+ B-cell lymphoma line). Chromium release data is shown of the effectors against different targets. JOVI-1 CAR T-cells kill Jurkats but not Raji cells or Jurkats with TCR knocked out.

FIG. 16: Self-Purging of JOVI-1 CAR T-cell cultures. Since T-cells comprise of approximately equal numbers of T-cells which are either TRBC1 or TRBC2 positive, it is possible that after introduction of CARs a certain amount of “fratricide” or self-purging of the culture may occur. It was demonstrated that this was the case. In this example, CAR T-cells were stained after transduction and analysed by flow-cytometry. Comparing mock-transduced versus transduced, one can observe that the T-cell population loses TRBC1 positive T-cells.

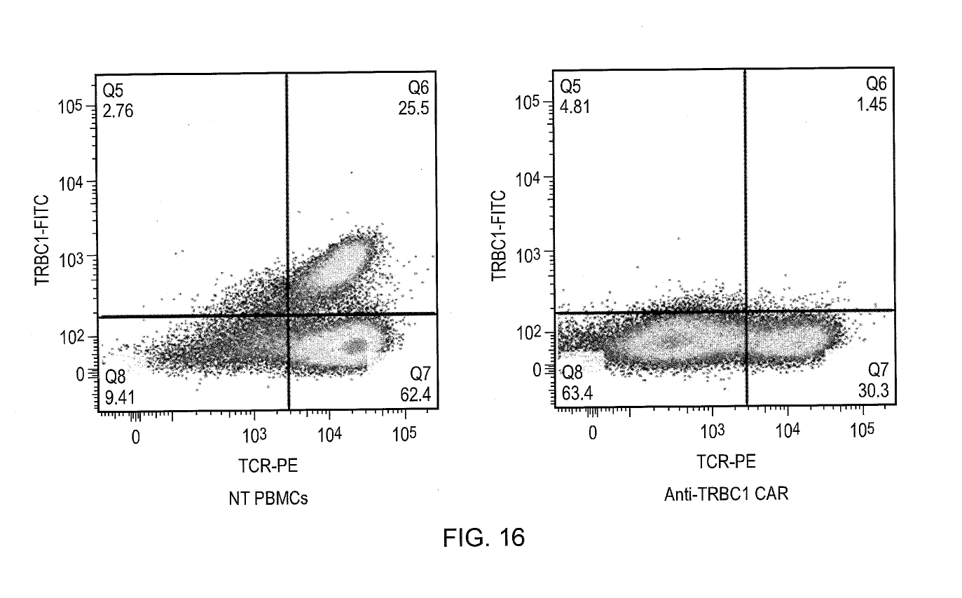

FIG. 17: Investigating the clonality of T-cell large granular Leukaemia (T-LGL)—Patient A

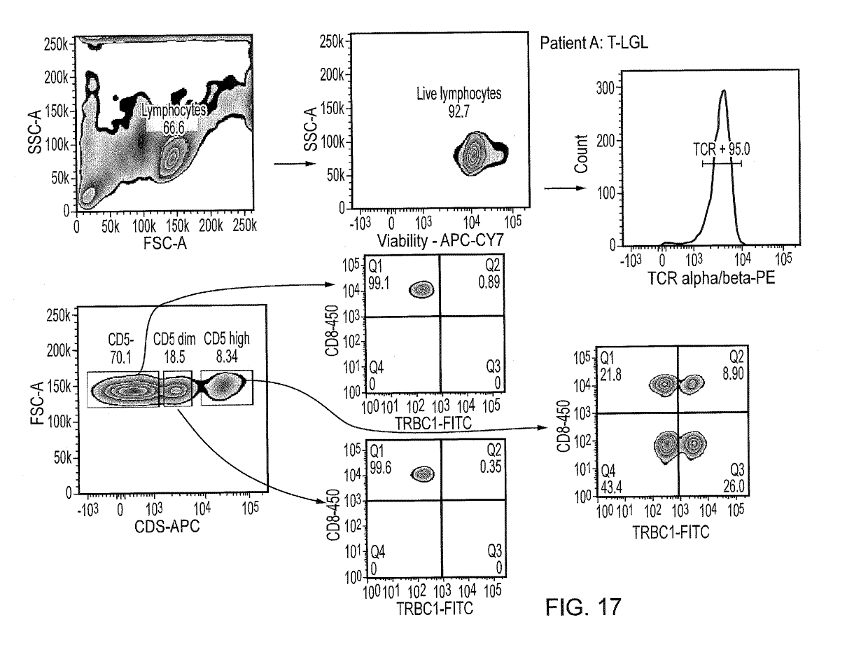

FIG. 18: Investigating the clonality of T-cell large granular Leukaemia (T-LGL)—Patient B

FIG. 19: Investigating the clonality of T-cell large granular Leukaemia (T-LGL)—Patient C

FIG. 20: Investigating the clonality of polyclonal T-cell lymphoma (PCTL)—Patient D

FIG. 21:TRBC peptide phage selection strategies. A) Two rounds of solid-phase phage display selections on TRBC peptides directly or indirectly immobilised on a surface. B) Three rounds of solution-phase phage display selections on biotinylated TRBC peptides.

FIG. 22: Analysis of polyclonal phage outputs from TRBC peptide phage display selections. TRF binding assay using polyclonal phage from solid phase selections carried out on TRBC peptides directly immobilised as BSA/OA conjugates (A), solid phase selections on TRBC peptides immobilised on streptavidin/neutravidin (B) and from solution phase selections (C).

FIG. 23: Schematic representation of pSANG10-3F vector. Gene encoding single chain antibody (scFv) is cloned at Ncol/Notl site downstream of T7 promoter and pelB leader (for periplasmic translocation). The vector also contains a C-terminal hexa-histidine tag (His6) for purification and tri-FLAG tag detection.

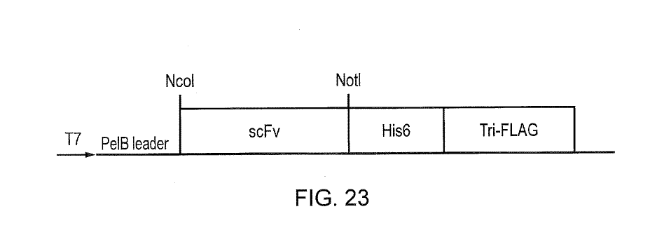

FIG. 24: Primary screening for TRBC1 and TRBC2 specific binders. Binding of 94 scFvs from TRBC1 (A) and TRBC2 (B) selections to biotinylated TRBC1 and TRBC2 (0.5 μg/ml) immobilised on neutravidin (10 μg/ml) coated Nunc Maxisorp™ 96 well plates. The scFv binding to the immobilised peptides was detected using an anti-FLAG antibody conjugated to europium.

FIG. 25: Binding of polyclonal antibody sera from rabbit #13174 immunized with TRBC1 against TRBC1 and TRBC2 peptides. (A) After 3rd immunization (B) After 3rd immunization and purification of TRBC1 specific antibodies.

FIG. 26: Binding of polyclonal antibody sera from rabbit #17363 immunized with TRBC2 peptide against TRBC1 and TRBC2 peptides. (A) After 3rd immunization (B) After 3rd immunization and purification of TRBC2 specific antibodies.

FIG. 27: Binding of polyclonal antibody sera from rabbit #17364 immunized with TRBC2 peptide against TRBC1 and TRBC2 peptides. (A) After 3rd immunization (B) After 3rd immunization and purification of TRBC2 specific antibodies.

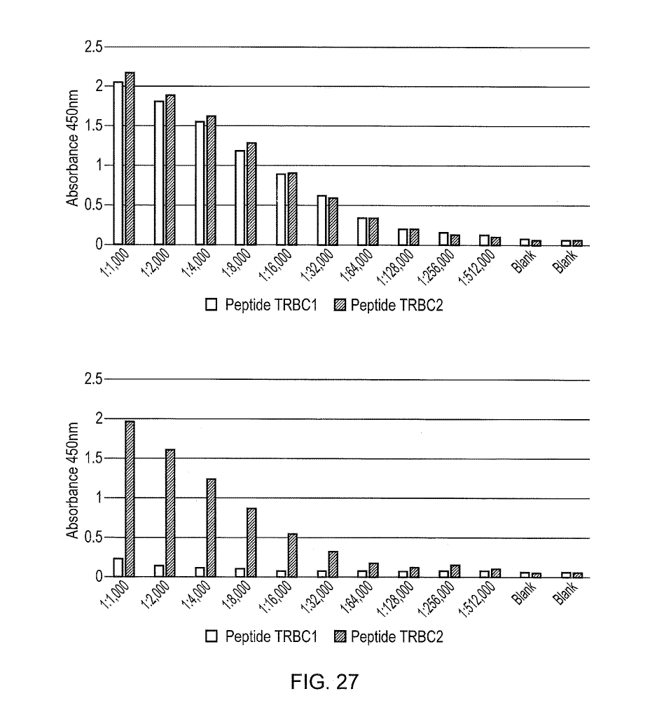

### SUMMARY OF ASPECTS OF THE INVENTION

The present inventors have devised a method whereby it is possible to deplete malignant T-cells in a subject, without affecting a significant proportion of healthy T cells. In particular, they have developed TRBC1 and TRBC2-specific chimeric antigen receptors (CARs) for use in the treatment of T-cell malignancies.

Thus in a first aspect the present invention provides a chimeric antigen receptor (CAR) which comprises an antigen-binding domain which selectively binds TCR beta constant region 1 (TRBC1) or TRBC2.

In a first embodiment of the first aspect of the invention there is provided a CAR which selectively binds TRBC1. In this embodiment, the CAR may comprise an antigen-binding domain which has a variable heavy chain (VH) and a variable light chain (VL) which comprise the following complementarity determining regions (CDRs):

VH CDR1: SEQ ID No. 7;

VH CDR2: SEQ ID No. 8;

VH CDR3: SEQ ID No. 9;

VL CDR1: SEQ ID No. 10;

VL CDR2: SEQ ID No. 11; and

VL CDR3: SEQ ID No. 12.

The CAR may comprise an antigen-binding domain which comprises a variable heavy chain (VH) having the sequence shown as SEQ ID No. 1 and a variable light chain (VL) having the sequence shown as SEQ ID No. 2.

The CAR may comprise an antigen-binding domain which comprises an scFv having the amino acid sequence shown as SEQ ID No. 3.

The CAR may comprise an amino acid sequence selected from SEQ ID No. 33, 34 and 35.

The CAR may comprise a VH CDR3 and/or a VL CDR3 from those listed in Table 1.

The CAR may comprise an antibody or functional fragment thereof which comprises:
(i) the heavy chain CDR3 and/or the light chain CDR3;
(ii) heavy chain CDR1, CDR2 and CDR3 and/or light chain CDR1, CDR2 and CDR3; or
(iii) the variable heavy chain (VH) and/or the variable light chain (VL); from one of the scFvs shown as SEQ ID No. 13 to 22.

In a second embodiment of the first aspect of the invention there is provided a CAR which selectively binds TRBC2.

In connection with this embodiment, the CAR may comprise a VH CDR3 and/or a VL CDR3 from those listed in Table 2.

The CAR may comprise an antibody or functional fragment thereof which comprises:
(i) the heavy chain CDR3 and/or the light chain CDR3;
(ii) heavy chain CDR1, CDR2 and CDR3 and/or light chain CDR1, CDR2 and CDR3; or
(iii) the variable heavy chain (VH) and/or the variable light chain (VL);
   from one of the scFvs shown as SEQ ID No. 23 to 32.

In a second aspect, the present invention provides a nucleic acid sequence encoding a CAR according to the first aspect of the invention.

In a third aspect, there is provided a vector which comprises a nucleic acid sequence according to the second aspect of the invention.

In a fourth aspect, there is provided a cell which comprises a CAR according to the first aspect of the invention. The cell may be a cytolytic immune cell, such as a T-cell or natural killer (NK) cell.

In a fifth aspect there is provided a method for making a cell according to the fourth aspect of the invention which comprises the step of transducing or transfecting a cell with a nucleic acid sequence according to the second aspect of the invention or a vector according to the third aspect of the invention.

In a sixth aspect there is provided a cell according to the fourth aspect of the invention for use in a method for treating a T-cell lymphoma or leukaemia in a subject which comprises the step of
administrating the cell comprising the TCRB1 or TCRB2 selective CAR to the subject, to cause selective depletion of the malignant T-cells, together with normal T-cells expressing the same TRBC as the malignant T-cells, but not to cause depletion of normal T-cells expressing the TRBC not expressed by the malignant T-cells.

The method may also comprise the step of investigating the TCR beta constant region (TCRB) of a malignant T cell from the subject to determine whether it expresses TRBC1 or TRBC2.

There is also provided a method for treating a T-cell lymphoma or leukaemia in a subject which comprises the step of administering a TCRB1 or TCRB2 selective agent to the subject, wherein the agent causes selective depletion of the malignant T-cells, together with normal T-cells expressing the same TRBC as the malignant T-cells, but does not cause depletion of normal T-cells expressing the TRBC not expressed by the malignant T-cells.

In a first embodiment of this aspect of the invention, the agent is a TCRB1 selective agent. In a second embodiment of this aspect of the invention, the agent is a TRBC2 selective agent.

The method may also comprise the step of investigating the TCR beta constant region (TRBC) of a malignant T-cell to determine whether it expresses TRBC1 or TRBC2, prior to the administration step.

The agent may be a depleting monoclonal antibody or a fragment thereof. The agent may be a conjugated antibody, which may comprise a chemotherapeutic entity.

The agent may be a bispecific T-cell engager. The agent may be a chimeric antigen receptor (CAR) expressing T-cell. The CAR may comprise an amino acid sequence selected from the group consisting of SEQ ID No. 33, 34 and 35.

The agent may comprise the JOVI-1 antibody or a functional fragment thereof.

The agent may comprise an antibody or a functional fragment thereof having a variable heavy chain (VH) and a variable light chain (VL) which comprise the following complementarity determining regions (CDRs):

VH CDR1: SEQ ID No. 7;

VH CDR2: SEQ ID No. 8;

VH CDR3: SEQ ID No. 9;

VL CDR1: SEQ ID No. 10;

VL CDR2: SEQ ID No. 11; and

VL CDR3: SEQ ID No. 12.

The agent may comprise an antibody of functional fragment thereof which comprises a variable heavy chain (VH) having the amino acid sequence shown as SEQ ID No. 1 and a variable light chain (VL) having the amino acid sequence shown as SEQ ID No. 2.

The agent may comprise an ScFv having the amino acid sequence shown as SEQ ID No. 3. The agent may be provided as a pharmaceutical composition.

The T-cell lymphoma or leukaemia may be selected from peripheral T-cell lymphoma, not otherwise specified (PTCL-NOS); angio-immunoblastic T-cell lymphoma (AITL), anaplastic large cell lymphoma (ALCL), enteropathy-associated T-cell lymphoma (EATL), hepatosplenic T-cell lymphoma (HSTL), extranodal NK/T-cell lymphoma nasal type, cutaneous T-cell lymphoma,primary cutaneous ALCL, T cell prolymphocytic leukaemia and T-cell acute lymphoblastic leukaemia.

The present invention also provides an agent for use in treating a T-cell lymphoma or leukaemia according to such a method.

The present invention also provides a kit comprising an agent for use as defined above.

The present invention also provides the use of an agent in the manufacture of a medicament for treatment of a T-cell lymphoma or leukaemia according to the above method.

The present invention also provides a method for diagnosing a T-cell lymphoma or leukaemia in a subject which comprises the step of determining the percentage of total T-cells in a sample which are TRBC1 or TRBC2 positive.

A percentage of TRBC1 or TRBC2 positive T-cells which is greater than about 80% may indicate the presence of a T-cell lymphoma or leukaemia.

The sample may be a peripheral blood sample or a biopsy.

The agent which binds total T-cells may bind CD3.

### DETAILED DESCRIPTION

The present invention provides agents, such as chimeric antigen receptors (CARs) which selectively bind TRBC1 or TRBC2. Such agents are useful in methods for treating a T-cell lymphoma or leukaemia in a subject. T cell malignancies are clonal, so they either express TRBC1 or TRBC2. By administering a TCRB1 or TCRB2 selective agent to the subject, the agent causes selective depletion of the malignant T-cells, together with normal T-cells expressing the same TRBC as the malignant T-cells, but does not cause depletion of normal T-cells expressing the TRBC not expressed by the malignant T-cells.

TCR β Constant Region (TRBC)

The T-cell receptor (TCR) is expressed on the surface of T lymphocytes and is responsible for recognizing antigens bound to major histocompatibility complex (MHC) molecules. When the TCR engages with antigenic peptide and MHC (peptide/MHC), the T lymphocyte is activated through a series of biochemical events mediated by associated enzymes, co-receptors, specialized adaptor molecules, and activated or released transcription factors.

The TCR is a disulfide-linked membrane-anchored heterodimer normally consisting of the highly variable alpha (α) and beta (β) chains expressed as part of a complex with the invariant CD3 chain molecules. T-cells expressing this receptor are referred to as α:β (or αβ) T-cells (˜95% total T-cells). A minority of T-cells express an alternate receptor, formed by variable gamma (γ) and delta (δ) chains, and are referred to as γδ T-cells (˜5% total T cells).

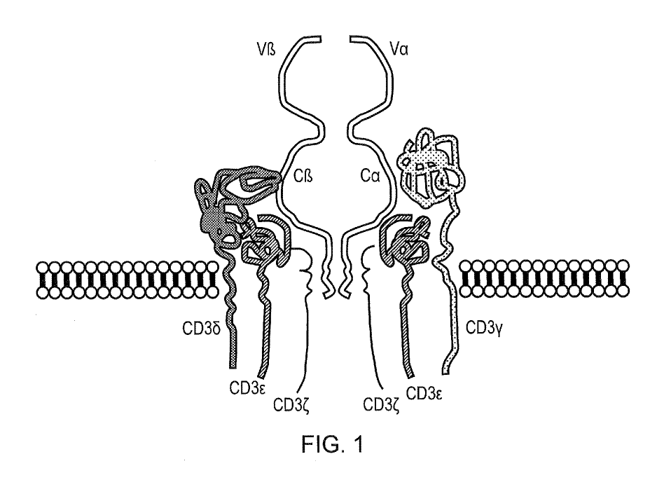

Each α and β chain is composed of two extracellular domains: Variable (V) region and a Constant (C) region, both of Immunoglobulin superfamily (IgSF) domain forming antiparallel β-sheets. The constant region is proximal to the cell membrane, followed by a transmembrane region and a short cytoplasmic tail, while the variable region binds to the peptide/MHC complex (see FIG. 1). The constant region of the TCR consists of short connecting sequences in which a cysteine residue forms disulfide bonds, which forms a link between the two chains.

The variable domains of both the TCR α-chain and β-chain have three hypervariable or complementarity determining regions (CDRs). The variable region of the β-chain also has an additional area of hypervariability (HV4), however, this does not normally contact antigen and is therefore not considered a CDR.

The TCR also comprises up to five invariant chains γ,δ,ε (collectively termed CD3) and ζ. The CD3 and ζ subunits mediate TCR signalling through specific cytoplasmic domains which interact with second-messenger and adapter molecules following the recognition of the antigen by αβ or γδ. Cell-surface expression of the TCR complex is preceded by the pair-wise assembly of subunits in which both the transmembrane and extracellular domains of TCR α and β and CD3 γ and δ play a role

TCRs are therefore commonly composed of the CD3 complex and the TCR α and β chains, which are in turn composed of variable and constant regions (FIG. 1).

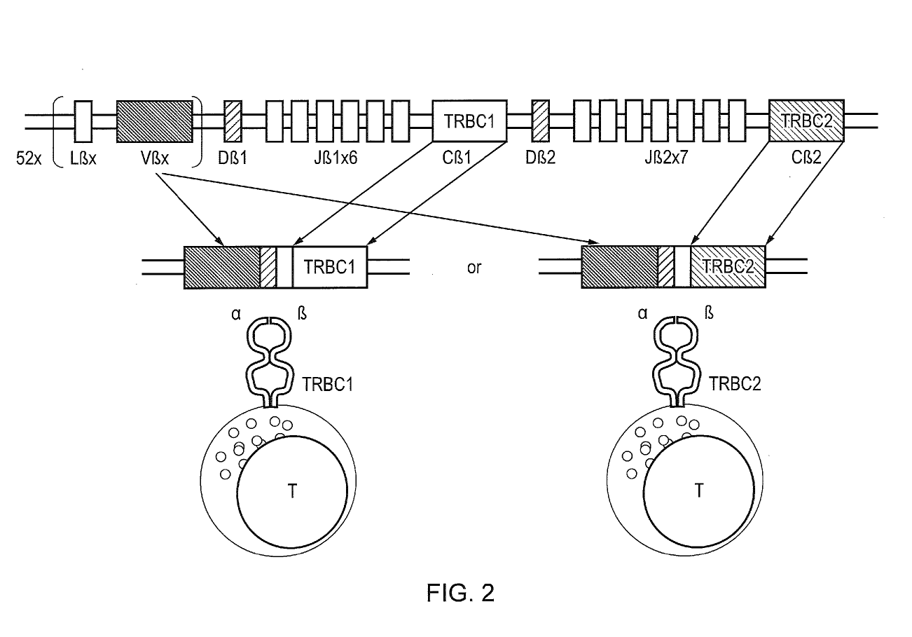

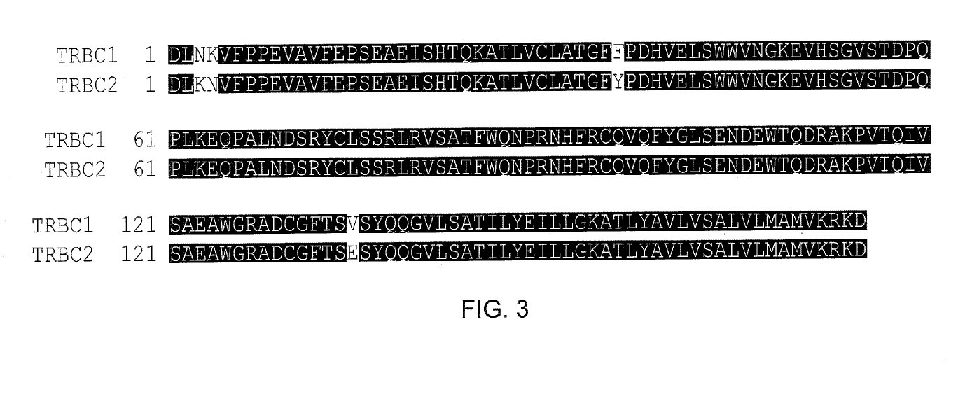

The locus (Chr7:q34) which supplies the TCR β-constant region (TRBC) has duplicated in evolutionary history to produce two almost identical and functionally equivalent genes: TRBC1 and TRBC2 (FIG. 2), which differ by only 4 amino acid in the mature protein produced by each (FIG. 3). Each TCR will comprise, in a mutually exclusive fashion, either TRBC1 or TRBC2 and as such, each αβ T-cell will express either TRBC1 or TRBC2, in a mutually exclusive manner.

The present inventors have determined that, despite the similarity between the sequence of the TRBC1 and TRBC2, it is possible to discriminate between them. The inventors have also determined that amino acid sequences of TRBC1 and TRBC2 can be discriminated whilst in situ on the surface of a cell, for example a T-cell.

Malignant Cells

The term ‘malignant’ is used herein according to its standard meaning to refer to a cell which is not self-limited in its growth, may be capable of invading into adjacent tissues and may be capable of spreading to distant tissue. As such the term ‘malignant T cell’ is used herein to refer to a clonally expanded T cell in the context of a lymphoma or leukaemia.

The method of the present invention involves determining the TRBC of a malignant T-cell. This may be performed using methods known in the art. For example it may be determined by PCR, western blot, flow cytometry or fluorescent microscopy methods.

Once the TRBC expressed by a malignant T-cell has been determined, the appropriate TRBC1 or TRBC2 selective agent is administered to the subject. The ‘appropriate TRBC selective agent’ means that where the malignant T-cell is determined to express TRBC1, a TRBC1 selective agent is administered, whereas where the malignant T-cell is determined to express TRBC2, a TRBC2 selective agent is administered.

Selective Agent

The selective agent binds to either TRBC1 or TRBC2 in a mutually exclusive manner.

As stated above, each αβ T-cell expresses a TCR which comprises either TRBC1 or TRBC2. In a clonal T-cell disorder, such as a T-cell lymphoma or leukaemia, malignant T-cells derived from the same clone will all express either TRBC1 or TRBC2.

Thus the present method comprises the step of administering a TRBC1 or TRBC2 selective agent to the subject, wherein the agent causes selective depletion of the malignant T-cells, together with normal T-cells which express the same TRBC as the malignant T-cells, but does not cause significant depletion of normal T-cells expressing the other TRBC from the malignant T-cells.

Because the TRBC selective agent does not cause significant depletion of normal T-cells expressing the other TRBC from the malignant T-cells it does not cause depletion of the entire T-cell compartment. Retention of a proportion of the subject's T-cell compartment (i.e. T-cells which do not express the same TRBC as the malignant T-cell) results in reduced toxicity and reduced cellular and humoral immunodeficiency, thereby reducing the risk of infection.

Administration of a TRBC1 selective agent according to the method of the present invention may result in a 5, 10, 20, 50, 75, 90, 95 or 99% depletion, i.e. reduction in the number of T-cells expressing TRBC1.

Administration of a TRBC2 selective agent according to the method of the present invention may result in a 5, 10, 20, 50, 75, 90, 95 or 99% depletion, i.e.reduction in the number of T-cells expressing TRBC2.

A TRBC1 selective agent may bind TRBC1 with an at least 2-fold, 4-fold, 5-fold, 7-fold or 10-fold greater affinity that TRBC2. Likewise, a TRBC2 selective agent may bind TRBC2 with an at least 2-fold, 4-fold, 5-fold, 7-fold or 10-fold greater affinity that TRBC1.

A TRBC1 selective agent causes depletion of a greater proportion of TRBC1-expressing T-cells in cell population than TRBC2-expressing cell. For example, the ratio of depletion of TRBC1-expressing T-cells to TRBC2-expressing cells may be at least 60%:40%, 70%:30%, 80%:20%, 90%:10% or 95%:5%. Likewise, a TRBC2 selective agent causes depletion of a greater proportion of TRBC1-expressing T-cells in cell population than TRBC2-expressing cell. For example, the ratio of depletion of TRBC2-expressing T-cells to TRBC1-expressing cells may be at least 60%:40%, 70%:30%, 80%:20%, 90%:10% or 95%:5%.

Using the method of the invention, malignant T-cells are deleted in a subject, without affecting a significant proportion of healthy T cells. By “a significant proportion” it is meant that a sufficient proportion of T cells expressing the TRBC different from the maltgnant T cells survive in order to maintain T-cell function in the subject. The agent may cause depletion of less than 20%, 15%, 10% or 5% of the T-cell population expressing the other TCRB.

The selective agent may be selective for either TRBC1 or TRBC2 because it discriminates residues as listed below:
(i) N from K at position 3 of the TRBC;
(ii) K from N at position 3 of the TRBC;
(iii) K from N at position 4 of the TRBC;
(iv) N from K at position 4 of the TRBC;
(v) F from Y at position 36 of the TRBC;
(vi) Y from F at position 36 of the TRBC;
(vii) V from E at position 135 of the TRBC; and/or
(viii) E from V at position 135 of the TRBC.

The selective agent may discriminate any combination of the differences above differences.

Antibody

The agent used in the method of the present invention may be a depleting monoclonal antibody (mAb) or a functional fragment thereof, or an antibody mimetic.

The term ‘depleting antibody’ is used in the conventional sense to relate to an antibody which binds to an antigen present on a target T-cell and mediates death of the target T-cell. The administration of a depleting antibody to a subject therefore results in a reduction/decrease in the number of cells within the subject which express the target antigen.

As used herein, “antibody” means a polypeptide having an antigen binding site which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′2, Fv, single chain Fv (ScFv) fragment or Nanobody. The antibody may be a bifunctional antibody. The antibody may be non-human, chimeric, humanised or fully human.

The antibody may therefore be any functional fragment which retains the antigen specificity of the full antibody.

TRBC1-Selective Antibodies

The agent for use in the method of the present invention may comprise an antibody or a functional fragment thereof having a variable heavy chain (VH) and a variable light chain (VL) which comprises one or more of the following complementarity determining regions (CDRs):

(SEQ ID No. 7)
VH CDR1: GYTFTGY;
(SEQ ID No. 8)
VH CDR2: NPYNDD;
(SEQ ID No. 9)
VH CDR3: GAGYNFDGAYRFFDF;
(SEQ ID No. 10)
VL CDR1: RSSQRLVHSNGNTYLH;
(SEQ ID No. 11)
VL CDR2: RVSNRFP;
and
(SEQ ID No. 12)
VL CDR3: SQSTHVPYT.

The one or more CDRs each independently may or may not comprise one or more amino acid mutations (eg substitutions) compared to the sequences given as SEQ ID No. 7 to 12, provided that the resultant antibody retains the ability to selectively bind to TRBC1.

Studies have shown that CDRs L3 and H3 are prevalently responsible for high energy interactions with the antigen, so the antibody or functional fragment thereof, may comprise VH CDR3 and/or VL CDR3 as outlined above.

Using phage display, several additional antibody binding domains have been identified which are highly selective for binding TRBC1 over TRBC2, as described in Example 12.

The agent may comprise an antibody or a functional fragment thereof having a variable heavy chain (VH) and/or a variable light chain (VL) which comprises one or more of the complementarity determining regions (CDR3s) shown in the Table 1.

TABLE 1

| Clone ID | VH germline ID | VL germline ID | Heavy CDR3 | Light CDR3 |
|---|---|---|---|---|
| CP_01_E09 | Vh3_DP-46_(3-30.3) | Vk1_DPK1_(O18, O8) | AHNSSSWSF . . . DY | QQYDNLP . . . LT |
| CP_01_D12 | Vh3_DP-46_(3-30.3) | Vk1_L12 | GGDTYGFL . . . DN | QQFNAYP . . . LT |
| CP_01_D10 | Vh3_DP-49_(3-30.5) | Vk1_DPK9_(O12, O2) | GGGSFGAF . . . DI | QQYNSYP . . . LT |
| CP_01_C08 | Vh3_DP-46_(3-30.3) | Vk1_DPK1_(O18, O8) | GYSSSWYL . . . DY | QQYDNLP . . . LT |
| CP_01_C11 | Vh1_DP-8,75_(1-02) | Vlambda6_6a | GGAG . . . WN | QSHDSSN . . . VV |
| CP_01_F03 | Vh3_DP-46_(3-30.3) | Vk1_DPK1_(O18, O8) | GYXASSWSQ . . . GL | QQYDNLP . . . PT |
| CP_01_E07 | Vh3_DP-49_(3-30.5) | Vk2_DPK12_(A2) | DLGGSGGAF . . . DI | MQSIQL . . . YT |
| CP_01_D03 | Vh3_DP-49_(3-30.5) | Vk3_DPK21_(L2) | NKQYGM . . . DV | QQYHRWP . . . LT |
| CP_01_F06 | Vh3_DP-49_(3-30.5) | Vk4_DPK24_(B3) | DDGAM . . . RY | QQYYDSP . . . YT |
| CP_01_F02 | Vh3_DP-46_(3-30.3) | Vk1_A30 | AGYSYA . . . DY | LQHNSYP . . . LT |
| CP_02_C03 | Vh3_DP-49_(3-30.5) | Vk4_DPK24_(B3) | GGRYSSNYF . . . DY | QQYFGT . . . PT |
| CP_02_D10 | Vh3_DP-50_(3-33) | Vk3_L16 | VGEGSAM . . . DV | QQYNDWP . . . LT |
| CP_02 B01 | Vh3_DP-46_(3-30.3) | Vlambda6_6a | VSSHYDSSGYYAGGF . . . DY | QSFDTNSL . . . WV |
| CP_02_D02 | Vh3_DP-49_(3-30.5) | Vk1_DPK1_(O18, O8) | GRDSSSWSP . . . AY | QQYDNLP . . . LT |
| CP_02_A02 | Vh3_DP-49_(3-30.5) | Vlambda2_DPL11_(2a2) | VTTYSGLDF . . . DY | SSYTSSST . . . VV |
| CP_02_D04 | Vh3_DP-46_(3-30.3) | Vk1_DPK9_(O12, O2) | KGAVVVPGAL . . . DY | QQYNSYP . . . LT |
| CP_01_E10 | Vh3_DP-49_(3-30.5) | Vk1_DPK9_(O12, O2) | NSLYGGNSA . . . DL | QQTFTTP . . . IT |
| CP_01_H08 | Vh3_DP-49_(3-30.5) | Vk1_DPK1_(O18, O8) | DGGGGRF . . . DY | QQYDNLP . . . LT |
| CP_01_F11 | Vh3_DP-46_(3-30.3) | Vlambda6_6a | GGGALGRGM . . . DV | QSYDTNN . . . VV |
| CP_0l_F09 | Vh5_DP-73_(5-51) | Vk1_DPK9_(O12, O2) | LLRSGGQSYAF . . . DI | QQSYSTP . . . LT |
| CP_02_D05 | Vh3_DP-46_(3-30.3) | Vk1_DPK1_(O18, O8) | GYSSSWSF . . . DY | QQYDNLP . . . IT |
| CP_02_A09 | Vh3_DP-46_(3-30.3) | Vk1_DPK1_(O18, O8) | AGSSGWTL . . . DY | QQYDNLP . . . LT |
| CP_02_D03 | Vh3_DP-46_(3-30.3) | Vk1_DPK1_(O18, O8) | DKGWGF . . . DY | QQYDNLP . . . IT |
| CP_02_C11 | Vh5_DP-73_(5-51) | Vlambda6_6a | LGVVRGVMKGF . . . DY | QSYDSSN . . . VV |
| CP_01_H10 | Vh3_DP-49_(3-30.5) | Vk1_DPK1_(O18, O8) | SSYSSSWGM . . . DV | QQYDNLP . . . LT |
| CP_02 C04 | Vh3_DP-49_(3-30.5) | Vk1_DPK9_(O12, O2) | ANSWSAGGM . . . DV | QQYDDLP . . . LT |
| CP_01_G03 | Vh3_DP-46_(3-30.3) | Vk2_DPK13_(O11, O1) | ERGRGYSYM . . . DV | MQRIEFP . . . LT |
| CP_01_G06 | Vh3_DP-46_(3-30.3) | Vlambda6_6a | VARGIHDAF . . . DI | QSYDNTRH . . . WV |
| CP_01_D06 | Vh1_DP-8,75_(1-02) | Vlambda6_6a | RHGM . . . DV | QSYDSSN . . . VV |
| CP_02_B03 | Vh5_DP-73_(5-51) | Vlambda6_6a | FDSSGYYY . . . DY | QSYDSSN . . . VV |
| CP_02_A12 | Vh3_DP-46_(3-30.3) | Vk3_DPK21_(L2) | DLVTTGAF . . . DT | QQHNDWP . . . LT |
| CP_01_H03 | Vh3_DP-49_(3-30.5) | Vlambda6_6a | AIRVSGTPENGF . . . DV | QSYHSSNI . . . WV |
| CP_01_G08 | Vh6_DP-74_(6-1) | Vk2_DPK16_(A23) | VRITHGM . . . DV | MQATHFP . . . QT |
| CP_0l_A06 | Vh3_DP-49_(3-30.5) | Vk4_DPK24_(B3) | GKLAF . . . DI | QQYYSTP . . . YT |
| CP_02_A04 | Vh3_DP-49_(3-30.5) | Vlambda6_6a | NGDSSGYHTSPNWYF . . . XL | QSYDDSNY . . . WV |
| CP_01_E08 | Vh3_DP-49_(3-30.5) | Vlambda2_2c | VSTDSSSM . . . DV | SSYAGSNTL . . . FV |
| CP_01_A08 | Vh3_DP-49_(3-30.5) | Vk1_DPK6_(L19) | TSQDPGAF . . . DI | QQANSFP . . . LT |
| CP_Ol_D01 | Vh3_DP-49_(3-30.5) | Vlambda6_6a | AESGVYSSNGM . . . DV | QSYDSSI . . . WV |
| CP_02_A07 | Vh3_DP-50_(3-33) | Vlambda6_6a | VDRVRSGM . . . DV | QSYDSIH . . . WV |
| CP_02_B08 | Vh3_DP-49_(3-30.5) | Vlambda6_6a | IGQYCSSTSCYM . . . DV | QSYDSSTH . . . WV |
| CP_01_D09 | Vh3_DP-49_(3-30.5) | Vk1_DPK1_(O18, O8) | DLGGSGGAF . . . DI | QQYDNLP . . . IT |
| CP_01_G07 | Vh3_DP-49_(3-30.5) | Vlambda6_6a | DSDAGVF . . . DL | QSFTSSTL . . . YV |
| CP_01_A05 | Vh3_DP-49_(3-30.5) | Vk3_DPK21_(L2) | ASIVASGAF . . . DI | QQYNKWP . . . LT |
| CP_02_A08 | Vh3_DP-49_(3-30.5) | Vlambda6_6a | AGGSNAF . . . DI | QSYDDSNY . . . WV |
| CP_02_D07 | Vh3_DP-49_(3-30.5) | Vlambda6_6a | VSTDSYGRQNWF . . . DP | QSYDSSNH . . . WV |
| CP_01_C04 | Vh3_DP-49 (3-30.5) | Vk1_DPK9_(O12, O2) | QYTSGRLAYYYHYM . . . DV | QQSYSTP . . . RT |
| CP_01_A07 | Vh1_DP-8,75_(1-02) | Vlambda3_DPL16_(3l) | GIRGAF . . . DI | NSRDSSGNPN . . . WV |
| CP_01_H02 | Vh3_DP-49_(3-30.5) | Vlambda6_6a | VGYSTTQL . . . DY | QSYDSSNL . . . WV |
| CP_01_F10 | Vh3_DP-49_(3-30.5) | Vlambda6_6a | MAGSYYAF . . . DI | QSYDSSNH . . . WV |
| CP_02_C10 | Vh3_DP-49_(3-30.5) | Vlambda1_DPL2_(1c) | VGDYYDSSGYLDWYF . . . DL | AVWDDRLNG . . . WV |
| CP_02_B05 | Vh3_DP-49_(3-30.5) | Vk1_L12 | GSDTTSFVS . . . DY | QQYDSYS . . . LT |
| CP_01_G04 | Vh3_DP-49_(3-30.5) | Vlambda3_3p | AGHYYYYM . . . DV | QSADSSGTN . . . MV |
| CP_01_F08 | Vh3_DP-49_(3-30.5) | Vlambda6_6a | VTGYPDYYDSSGF . . . DY | QSYDSSNH . . . WV |
| CP_01_G05 | Vh3_DP-49_(3-30.5) | Vlambda6_6a | VEGGPPYYF . . . DH | QSYDTRNQ . . . WV |
| CP_02_A03 | Vh3_DP-49_(3-30.5) | Vk4_DPK24_(B3) | NGLDNYGM . . . DV | QQYYSTP . . . YT |
| CP_01_B09 | Vh5_DP-73_(5-51) | Vk1_DPK9_(O12, O2) | LGTTKRAF . . . DI | QQSYST . . . RT |
| CP_01_A10 | Vh3DP-49_(3-30.5) | Vlambda1_DPL3_(1g) | VYVDHEGM . . . DV | AAWDDSLF . . . WL |
| CP_01_H04 | Vh3_DP-49_(3-30.5) | Vk2_DPK13_(O11, O1) | WSGSGF . . . DY | MQRIEFP . . . LT |
| CP_02_B04 | Vh3 DP-49 (3-30.5) | Vk4_DPK24_(B3) | DFGWGGAF . . . DI | QQYYNTP . . . LI |
| CP_02_A05 | Vh5_DP-73_(5-51) | Vlambda6_6a | VVGGTQH . . . DY | QSYDSSI . . . VV |
| CP_01_F07 | Vh3_DP-49_(3-30.5) | Vlambda6_6a | NWLLYYGDPQNAF . . . DI | QSYDSTNL . . . WV |
| CP_01_H01 | Vh5_DP-73_(5-51) | Vk1_DPK9_(O12, O2) | LYFDWFADSQNAF . . . DI | QQSYSTP . . . LT |
| CP_01_G10 | Vh3_DP-49_(3-30.5) | Vlambda3_DPL16_(3l) | VGYQPLLYADYYF . . . DY | NSRDSSGNH . . . LV |
| CP_01_G11 | Vh3_DP-49_(3-30.5) | Vk1_DPK9_(O12, O2) | GAMGL . . . DY | QQSYSTP . . . FT |
| CP_01_G01 | Vh3_DP-49_(3-30.5) | Vlambda3_DPL16_(3l) | VYYLSDVHAF . . . DV | DSRDTRVNX . . . WI |
| CP_01_A12 | Vh3_DP-46_(3-30.3) | Vk1_DPK1_(O18, O8) | TERWLQF . . . DY | QQYDNL . . . PS |
| CP_01_H05 | Vh3_DP-49_(3-30.5) | Vk2_DPK15_(A19, A3) | NGDYAF . . . DY | MOALQTP . . . VT |
| CP 02_B07 | Vh3_DP-49_(3-30.5) | Vlambda6_6a | ASRYSGSYHF . . . DY | QSYDSSN . . . VV |
| CP_01_G09 | Vh3_DP-46_(3-30.3) | Vlambda2_DPL11_(2a2) | HGSQGGF . . . DI | SSYTSSST . . . LV |
| CP_02_C02 | Vh3_DP-49_(3-30.5) | Vk2_DPK15_(A19, A3) | VGYMGGM . . . DV | MQALQTPP . . . YT |
| CP_01_D05 | Vh3_DP-49_(3-30.5) | Vlambda6_6a | NTPGIAAAGP . . . DS | QSYDSTNH . . . WV |
| CP_01_D08 | Vh3_DP-49_(3-30.5) | Vlambda6_6a | VGTTIVTSF . . . DY | QSYDSANL . . . WV |
| CP_02_A11 | Vh3_DP-46_(3-30.3) | Vlambda6_6a | VGGPLNDAF . . . DI | QSFDENIS . . . WV |
| CP_02_D08 | Vh3_DP-49_(3-30.5) | Vk3_DPK22_(A27) | HSSGGAF . . . DI | HQSATSP . . . LT |
| | | Clone ID | TRBC1 binding | TRBC2 Binding |
| | | CP_01_E09 | 403248 | 318 |
| | | CP_01_D12 | 392753 | 298 |
| | | CP_01_D10 | 370612 | 306 |
| | | CP_01_C08 | 352814 | 426 |
| | | CP_01_C11 | 349231 | 622 |
| | | CP_01_F03 | 335086 | 306 |
| | | CP_01_E07 | 332307 | 394 |
| | | CP_01_D03 | 327666 | 452 |
| | | CP_01_F06 | 325058 | 286 |
| | | CP_01_F02 | 301955 | 508 |
| | | CP_02_C03 | 274905 | 374 |
| | | CP_02_D10 | 259096 | 517 |
| | | CP_02 B01 | 258840 | 341 |
| | | CP_02_D02 | 256223 | 393 |
| | | CP_02_A02 | 252590 | 385 |
| | | CP_02_D04 | 252076 | 493 |
| | | CP_01_E10 | 238172 | 679 |
| | | CP_01_H08 | 223591 | 381 |
| | | CP_01_F11 | 222976 | 481 |
| | | CP_0l_F09 | 217934 | 308 |
| | | CP_02_D05 | 212579 | 440 |
| | | CP_02_A09 | 202054 | 336 |
| | | CP_02_D03 | 199403 | 543 |
| | | CP_02_C11 | 189481 | 392 |
| | | CP_01_H10 | 179830 | 424 |
| | | CP_02 C04 | 172937 | 722 |
| | | CP_01_G03 | 168169 | 360 |
| | | CP_01_G06 | 166703 | 307 |
| | | CP_01_D06 | 162783 | 287 |
| | | CP_02_B03 | 158809 | 312 |
| | | CP_02_A12 | 152968 | 280 |
| | | CP_01_H03 | 151902 | 590 |
| | | CP_01_G08 | 137502 | 736 |
| | | CP_0l_A06 | 136525 | 354 |
| | | CP_02_A04 | 130318 | 385 |
| | | CP_01_E08 | 126690 | 545 |
| | | CP_01_A08 | 117913 | 270 |
| | | CP_Ol_D01 | 116603 | 204 |
| | | CP_02_A07 | 105730 | 496 |
| | | CP_02_B08 | 96003 | 795 |
| | | CP_01_D09 | 92079 | 282 |
| | | CP_01_G07 | 77222 | 313 |
| | | CP_01_A05 | 75698 | 705 |
| | | CP_02_A08 | 73410 | 295 |
| | | CP_02_D07 | 72274 | 367 |
| | | CP_01_C04 | 65702 | 286 |
| | | CP_01_A07 | 63917 | 238 |
| | | CP_01_H02 | 63410 | 266 |
| | | CP_01_F10 | 58027 | 372 |
| | | CP_02_C10 | 53460 | 488 |
| | | CP_02_B05 | 51460 | 315 |
| | | CP_01_G04 | 50811 | 354 |
| | | CP_01_F08 | 43115 | 562 |
| | | CP_01_G05 | 42289 | 409 |
| | | CP_02_A03 | 39382 | 322 |
| | | CP_01_B09 | 38766 | 803 |
| | | CP_01_A10 | 38613 | 298 |
| | | CP_01_H04 | 34030 | 305 |
| | | CP_02_B04 | 30975 | 348 |
| | | CP_02_A05 | 30140 | 309 |
| | | CP_01_F07 | 29443 | 331 |
| | | CP_01_H01 | 26847 | 349 |
| | | CP_01_G10 | 26520 | 360 |
| | | CP_01_G11 | 26087 | 292 |
| | | CP_01_G01 | 25464 | 423 |
| | | CP_01_A12 | 23458 | 331 |
| | | CP_01_H05 | 20298 | 322 |
| | | CP 02_B07 | 19598 | 217 |
| | | CP_01_G09 | 18725 | 449 |
| | | CP_02_C02 | 18320 | 468 |
| | | CP_01_D05 | 17240 | 299 |
| | | CP_01_D08 | 16499 | 291 |
| | | CP_02_A11 | 13370 | 329 |
| | | CP_02_D08 | 12277 | 566 |

Where the agent is a domain antibody it may comprise 3 CDRs, i.e. either VH CDR1-CDR3 or VL CDR1-CDR3.

The agent may comprise an antibody of functional fragment thereof which comprises a variable heavy chain (VH) having the amino acid sequence shown as SEQ ID No. 1 and a variable light chain (VL) having the amino acid sequence shown as SEQ ID No. 2.

SEQ_ID_1
Jovi-1 VH
EVRLQQSGPDLIKPGASVKMSCKASGYTFTGYVMHWVKQRPGQGLEWIGF
INPYNDDIQSNERFRGKATLTSDKSSTTAYMELSSLTSEDSAVYYCARGA
GYNFDGAYRFFDFWGQGTTLTVSS
SEQ_ID_2
Jovi-1 VL
DVVMTQSPLSLPVSLGDQASISCRSSQRLVHSNGNTYLHWYLQKPGQSPK
LLIYRVSNRFPGVPDRFSGSGSGTDFTLKISRVEAEDLGIYFCSQSTHVP
YTFGGGTKLEIKR

The agent may comprise an ScFv having the amino acid sequence shown as SEQ ID No. 3.

SEQ_ID_3
Jovi-1 scFv
EVRLQQSGPDLIKPGASVKMSCKASGYTFTGYVMHWVKQRPGQGLEWIGF
INPYNDDIQSNERFRGKATLTSDKSSTTAYMELSSLTSEDSAVYYCARGA
GYNFDGAYRFFDFWGQGTTLTVSSGGGGSGGGGSGGGGSDVVMTQSPLSL
PVSLGDQASISCRSSQRLVHSNGNTYLHWYLQKPGQSPKWYRVSNRFPGV
PDRFSGSGSGTDFTLKISRVEAEDLGIYFCSQSTHVPYTFGGGTKLEIKR

Alternatively, the agent may comprise an antibody or functional fragment thereof which comprises:
(i) the heavy chain CDR3 and/or the light chain CDR3;
(ii) heavy chain CDR1, CDR2 and CDR3 and/or light chain CDR1, CDR2 and CDR3; or
(iii) the variable heavy chain (VH) and/or the variable light chain (VL); from one of the scFvs shown as SEQ ID No. 13-22.

In the sequences shown as SEQ ID No. 13-22, the VH and VL portions of the sequence are shown in bold and the CDR1 and CDR2 sequences for the heavy and light chains are underlined. The CDR3 sequences for VH and VL are given in Table 1.

SEQ ID No. 13
(CP_01_E09)
QVQLVESGGGVVQPGRSLRLSCAASGFTFSSYAMHWVRQAPGKGLEWVAV
ISYDGSNKYYADSVKGRFTISRDNSKNTLYLQMNSLRAEDTAVYYCARAH
NSSSWSFDYWGQGTLVTVSSGGGGSGGGGSGGGASDIQMTQSPSSLSASV
GDRVTITCRASQSISSYLNWYQQKPGKAPKLLIYDASNLETGVPSRFSGS
GSGTDFTFTISSLQPEDIATYYCQQYDNLPLTFGGGTKVDIKRTAAA
SEQ ID No. 14
(CP_01_D12)
EVQLLESGGGVVQPGRSLRLSCAASGFTFSSYAMHWVRQAPGKGLEWVAV
ISYDGSNKYYADSVKGRFTISRDNSKNTLYLQMNSLRAEDTAVYYCAKGG
DTYGFLDNWGQGTMVTVSSGGGGSGGGGSGGGASDIQMTQSPSTLSASVG
DRVTITCRASQSISSWLAWYQQKPGKAPKLLIYKASSLESGVPSRFSGSG
SGTDFTLTISSLQPEDFATYYCQQFNAYPLTFGGGTKVEIKRTAAA
SEQ ID No. 15
(CP_01_D10)
QVQLVESGGGLVQPGRSLRLSCAASGFTFSSYAMHWVRQAPGKGLEWVAV
ISYDGSSKYYADSVKGRFTISRDNSKNTLYLQMNSLRAEDTAVYYCAKGG
GSFGAFDIWGQGTLVTVSSGGGGSGGGGSGGGASDIQMTQSPSSLSASVG
DRVTITCRASQSISRYLNWYQQKPGKAPNLLIYAASSLQSGVPSRFSGSG
SGTDFTLTISSLQPEDFATYYCQQYNSYPLTFGGGTKLEIKRTAAA
SEQ ID No. 16
(CP_01_C08)
EVQLLESGGGAVQPGRSLRLSCAASGFTFSSYAMHWVRQAPGKGLEWVAV
ISYD [symbol] RFTISRDNSKNTLYLQMNSLRAEDTAVYYCAS
GYSSSWYLDYWGQGTLVTVSSGGGGSGGGGSGGGASDIQMTQSPSSVSAS
VGDRVTITCQASQDISNYLNWYQQKPGKAPKLLIYDASNLETGVPSRFSG
SGSGTDFTFTISSLQPEDIATYYCQQYDNLPLTFGGGTKVEIKRTAAA
SEQ ID No. 17
(CP_01_C11)
QVQLVESGAEVKKPGASVKVSCKASGYTFTGYYMHWVRQAPGQGLEWMGR
INPNSGGTNYAQKFQGRVTMTRDTSISTAYMELSRLRSDDTAVYYCASGG
AGWNWGQGTMVTVSSGGGGSGGGGSGGGASNFMLTQPHSVSESPGKTATI
SCTRSSGSIASNYVQWYQQRPGSAPTTVIYEDNQRPFGVPDRFSGSIDSS
SNSASLTISGLKTEDEADYYCQSHDSSNVVFGGGTQLTVLGQPAA
SEQ ID No. 18
(CP_01_F03)
EVQLVESGGGVVQPGRSLRLSCAASGFTFSSYAMHWVRQAPGKGLEWVAV
ISYDGSNKYYADSVKGRFTISRDNSKNTLYLQMNSLRAEDTAVYYCARGY
?ASSWSQGLWGQGTLVTVSSGGGGSGGGGSGGGASDIQMTQSPSSLSASV
RDRVTITCQASQDISNYLNWYQQKPGKAPKLLIYDASNLETGVPSRFSGS
GSGTDFTFTISSLQPEDIATYYCQQYDNLPPTFGGGTKVEIKRTAAA
SEQ ID No. 19
(CP_01_E07)
QVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKDL
GGSGGAFDIWGQGTLVTVSSGGGGSGGGGSGGGASDIVMTQTPHSLSVTP
GQPASISCKSSQSLLYSDGKTYLYWYLQKPGQPPQLLIYEVSNRFSGVPD
RFSGSGSGTDFTLKISRVEAEDVGVYYCMQSIQLYTFGQGTKVDIKRTAA
A
SEQ ID No. 20
(CP_01_D03)
QVQLVESGGGVVQPGRSLRLSCAAPGFTFSSYGMHWVRQAPGKGLEWVAV
ISYDGSNKYYADSVKGRFTISRDNSKNTLYLQMNSLRAEDTAVYYCARNK
QYGMDVWGQGTLVTVSSGGGGSGGGGSGGGASDIVMTQSPATLSLAPGER
ATLSCRASQSVGSNLAWYQQKPGQAPSLLIYDASTRATGIPARFSGSGSG
TDFTLTISSLQSEDIAVYYCQQYHRWPLTFGGGTKVEIKRTAAA
SEQ ID No. 21
(CP_01_F06)
EVQLLESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKDD
GAMRYWGQGTMVTVSSGGGGSGGGGSGGGASDIQMTQSPDSLAVSLGERA
TINCKSSQSVLYSSNNKNYLAVVYQQKPGQPPKLLIYWASTRESGVPDRF
SGSGSGTDFTLTISSLQAEDVAVYYCQQYYDSPYTFGQGTKVDIKRTAAA
SEQ ID No. 22
(CP_01_F02)
QVQLVESGGGVVQPGRPLRLSCAASGFTFSSYAMHWVRQAPGKGLEWVAV
ISYDGSNKYYADSVKGRFTISRDNSKNTLYLQMNSLRAEDTAVYYCARAG
YSYADYWGQGTMVTVSSGGGGSGGGGSGGGASDIQMTQSPSSLSASVGDR
VTITCRASQGIRNDLGWYQQKPGKAPKRLIYAASSLQSGVPSRFSGSGSG
TEFTLTISSLQPEDFATYYCLQHNSYPLTFGGGTKVDIKRTAAA

TRBC2-Specific

Using phage display, several antibody binding domains have been identified which are highly selective for binding TRBC2 over TRBC1, as described in Example 12.

The agent may comprise an antibody or a functional fragment thereof having a variable heavy chain (VH) and/or a variable light chain (VL) which comprises one or more of the complementarity determining regions (CDR3s) shown in the Table 2.

TABLE 2

| | | | CDR3 | |
|---|---|---|---|---|
| Clone ID | VH dp number | VL dp number | group | Heavy CDR3 |
| CP_03_E05 | Vh3_DP-47_(3-23) | Vlambda6_6a | 38 | TRSSGAF . . . DI |
| CP_03_D05 | Vh1_DP-8,75_(1-02) | Vlambda3_DPL23_(3r) | 33 | PRGRGSAF . . . DI |
| CP_03_H06 | Vh3_DP-35_(3-11) | Vlambda6_6a | 20 | ARVGGM . . . DV |
| CP_03_C12 | Vh1_DP-8,75_(1-02) | Vk2_DPK12_(A2) | 27 | DTGPI . . . DY |
| CP_03_G02 | Vh1_DP-7_(1-46) | Vk1_DPK9_(O12,02) | 10 | GVWNSGSYLGF . . . D |
| CP_03_D04 | Vh3_DP-46_(3-30.3) | Vlambda6_6a | 28 | GGFTVPGGAF . . . DI |
| CP_03_F10 | Vh1_DP-8,75_(1-02) | Vlambda2_2c | 42 | FGERYAF . . . DI |
| CP_03_G09 | Vh6_DP-74_(6-1) | Vlambda3_3j | 34 | DQWLANYYYYGM . . . |
| CP_03_F09 | Vh1_DP-7_(1-46) | Vlambda6_6a | 8 | NRGGSYKSVGM . . . D |
| CP_03_D09 | Vh1_DP-15_(1-08) | Vlambda6_6a | 11 | VSSYYGM . . . DV |
| CP_03_F02 | Vh1_DP-15_(1-08) | Vlambda6_6a | 3 | APASSAH . . . DH |
| CP_02_E03 | Vh3_DP-46_(3-30.3) | Vk1_DPK9_(O12, O2) | 16 | QRGYYYGM . . . DV |
| C9_03_H07 | Vh3_DP-31_(3-09) | Vlambda2_DPL11_(2a2) | 14 | SSVAAGAF . . . DI |
| CP_03_C02 | Vh5_DP-73_(5-51) | Vlambda2_DPL10_(2b2) | 13 | LSGRGLGF . . . DY |
| CP_03_E09 | Vh1_DP-8,75_(1-02) | Vlambda2_DPL11_(2a2) | 5 | DHYF . . . DY |
| CP_03_D08 | Vh3_DP-46_(3-30.3) | Vlambda6_6a | 19 | SGRRVTAI . . . DY |
| CP_03_E11 | Vh1_DP-8,75_(1-02) | Vlambda3_3h | 37 | MGRYSSSW . . . NI |
| CP_03_B05 | Vh5_DP-73_(5-51) | Vlambda2_DPL10_(2b2) | 12 | HSRFGPAF . . . DI |
| CP_03_H02 | Vh1_DP-8,75_(1-02) | Vlambda3_DPL23_(3r) | 25 | DREAF . . . DI |
| CP_03_D02 | Vh5_DP-73_(5-51) | Vlambda2_DPL10_(2b2) | 7 | LRGRYSYGYSDAF . . . D |
| CP_03_E01 | Vh1_DP-8,75_(1-02) | Vlambda3_DPL16_(3l) | 36 | LLNAVTYAF . . . DI |
| CP_03_C11 | Vh1_DP-8,75_(1-02) | Vlambda2_DPL11_(2a2) | 2 | IGVIGGF . . . DY |
| CP_03_B12 | Vh3_DP-47_(3-23) | Vlambda6_6a | 9 | IEYSSSSPYF . . . DY |
| CP_03_E03 | Vh1_DP-7_(1-46) | Vlambda3_DPL16_(3l) | 18 | DLLPTIVTTTGAF . . . DI |
| CP_03_F01 | Vh1_DP-8,75_(1-02) | Vlambda3_DPL23_(3r) | 40 | DSGSYS . . . DY |
| CP_03_C01 | Vh6 DP-74_(6-1) | Vlambda3_3j | 31 | ASYPYYYYYYGM . . . D [symbol] |
| CP_03_G07 | Vh6 DP-74_(6-1) | Vlambda6_6a | 41 | ALGHF . . . DF |
| CP_03_E02 | Vh5_DP-73_(5-51) | Vlambda2_DPL11_(2a2) | 35 | FTIGSAL . . . YM |
| CP_03_C07 | Vh1_DP-8,75_(1-02) | Vlambda3_DPL23_(3r) | 17 | DASGY . . . DY |
| CP_03_H04 | Vh1_DP-8,75_(1-02) | Vlambda1_DPL5_(1b) | 1 | DLGTYYYGSGD . . . DY |
| CP_03_E06 | Vhl DP-8,75_(1-02) | Vlambda3_DPL16_(3l) | 39 | VGELLGAF . . . DI |
| CP_03_H03 | Vh1_DP-5_(1-24) | Vlambda1_DPL2_(1c) | 15 | GL . . . GV |
| CP_03_G11 | Vh5_DP-73_(5-51) | Vlambda2_DPL12_(2e) | 23 | HSGVGGLAF . . . DI |
| CP_03_G01 | Vh6_DP-74_(6-1) | Vk1_L9 | 4 | GGSIAAALAF . . . DI |
| CP_03_H01 | Vh1_DP-15_(1-08) | Vlambda3_DPL16_(3l) | 30 | VEYSRNGM . . . DV |
| CP_03_F11 | Vh1 DP-15_(1-08) | Vlambda6_6a | 22 | GRYN . . . LI |
| CP_03_C06 | Vh1_DP-14_(1-18) | Vlambda6_6a | 32 | LDYYYGM . . . DV |
| CP_03_D03 | Vh1_DP-15_(1-08) | Vlambda6_6a | 26 | GGLSSAF . . . DI |
| CP_03_G05 | Vh5_DP-73_(5-51) | Vlambda2_DPL12_(2e) | 6 | YGGGL . . . DV |
| CP_03_G12 | Vh3_DP-47_(3-23) | Vlambda2_DPL11_(2a2) | 21 | PDHLTVF . . . DY |
| CP_03_C10 | Vh1_DP-8,75_(1-02) | Vlambda3_DPL23_(3r) | 24 | VGYYGM . . . DV |
| CP_03_F04 | Vh1_DP-8,75_(1-02) | Vlambda3_DPL23_(3r) | 29 | YEGYAGF . . . DY |
| | CDR3 | | | |
| Clone ID | group | Light CDR3 | TRBC2 binding | TRBC1 binding |
| CP_03_E05 | 11 | HSYDSNNH . . . SV | 217270 | 1617 |
| CP_03_D05 | 27 | QAWDTNLG . . . G [symbol] | 212721 | 762 |
| CP_03_H06 | 19 | QSFDADNLH . . . V [symbol] | 167971 | 391 |
| CP_03_C12 | 5 | MQGIQLP . . . PT | 167371 | 789 |
| CP_03_G02 | 30 | QQSYSTP . . . LT | 151586 | 787 |
| CP_03_D04 | 13 | QSYDASN . . . VI | 143051 | 1210 |
| CP_03_F10 | 9 | SAYTGSN . . . YV | 139767 | 1683 |
| CP_03_G09 | 34 | QVWDSNS . . . W [symbol] | 138659 | 979 |
| CP_03_F09 | 36 | QSYDEVS . . . VV | 131852 | 889 |
| CP_03_D09 | 28 | QSYNSSNH . . . W [symbol] | 128690 | 544 |
| CP_03_F02 | 14 | QSYDSSH . . . VV | 127081 | 1507 |
| CP_02_E03 | 12 | QQSRSTP . . . LT | 122650 | 1979 |
| C9_03_H07 | 22 | SSYTSSST . . . WV | 120948 | 1233 |
| CP_03_C02 | 10 | SSYAGSSNL . . . WV | 100238 | 1904 |
| CP_03_E09 | 21 | NSYTRSST . . . LV | 99580 | 1011 |
| CP_03_D08 | 8 | QSYDDTN . . . VV | 92074 | 453 |
| CP_03_E11 | 3 | QAWDTNIG . . . GV | 91813 | 940 |
| CP_03_B05 | 23 | SSYAGSNN . . . YV | 88004 | 815 |
| CP_03_H02 | 3 | QAWDTNIG . . . GV | 87576 | 1829 |
| CP_03_D02 | 17 | SSYAGSST . . . FV | 84907 | 1096 |
| CP_03_E01 | 4 | NSRDSSGF . . . PV | 81606 | 498 |
| CP_03_C11 | 18 | SSYTSSS . . . IL | 78572 | 1079 |
| CP_03_B12 | 16 | QSYDSNNR . . . VL | 70734 | 1120 |
| CP_03_E03 | 7 | SSRDSSGNH . . . LV | 69661 | 356 |
| CP_03_F01 | 3 | QAWDTNIG . . . GV | 66921 | 1633 |
| CP_03_C01 | 29 | QVWDSSTAN . . . V | 58194 | 825 |
| CP_03_G07 | 32 | QSYDSSNHH . . . VY | 57147 | 1278 |
| CP_03_E02 | 26 | SSYAGNSN . . . LV | 52212 | 362 |
| CP_03_C07 | 3 | QAWDTNIG . . . GV | 43547 | 1074 |
| CP_03_H04 | 2 | GTWDSSLSAG . . . Q | 35180 | 1103 |
| CP_03_E06 | 35 | SSLDSNDNH . . . PI | 34777 | 917 |
| CP_03_H03 | 37 | AAWDDSLNG . . . Y | 33358 | 1405 |
| CP_03_G11 | 31 | SSYAGSST . . . YV | 30854 | 836 |
| CP_03_G01 | 20 | HQYDVYP . . . PT | 30762 | 1039 |
| CP_03_H01 | 6 | NSRDSSGNH . . . LV | 29826 | 1203 |
| CP_03_F11 | 15 | QSYDSSN . . . WV | 24172 | 1152 |
| CP_03_C06 | 33 | QSYDSSN . . . QV | 23031 | 937 |
| CP_03_D03 | 24 | QSYDSSN . . . VV | 22905 | 1283 |
| CP_03_G05 | 25 | SSYAGSYT . . . LV | 22037 | 813 |
| CP_03_G12 | 1 | SSYTPSS . . . VL | 20349 | 942 |
| CP_03_C10 | 3 | QAWDTNIG . . . GV | 18438 | 896 |
| CP_03_F04 | 3 | QAWDTNIG . . . GV | 13541 | 1047 |

[symbol]

indicates data missing or illegible when filed

The agent may comprise an antibody or functional fragment thereof which comprises:
(i) the heavy chain CDR3 and/or the light chain CDR3;
(ii) Heavy chain CDR1, CDR2 and CDR3 and/or light chain CDR1, CDR2 and CDR3; or
(iii) the variable heavy chain (VH) and/or the variable light chain (VL);
   from one of the scFvs shown as SEQ ID No. 23-32.

In the sequences shown as SEQ ID No. 23-32, the VH and VL portions of the sequence are shown in bold and the CDR1 and CDR2 sequences for the heavy and light chains are underlined. The CDR3 sequences for VH and VL are given in Table 2.

SEQ ID No. 23
(CP_03_E05) [symbol]
VQL [symbol] ESGGGVVQPGGSLRLSCAASGFTFSSYAMSWVRQAPGKGLEWVS
AISGSGGSTYYADSVKGRFSISRDNSKNTLYLQMNSLRAEDTAVYYCART
RSSGAFDIWGQGTLVTVSSGGGGSGGGGSGGGASNFMLTQPHSVSESPGK
TVTISCTRSSGSIASKYVQWYQQRPGSSPTTVIYEDNQRPSGVPDRFSGS
IDTSSNSASLTISGLRTEDEADYYCHSYDSNNHSVFGGGTKVTVLGQPA
A
SEQ ID No. 24
(CP_03_D05)
QVQLVESGAEVKKPGASVKVSCKASGYTFTGYYMHWVRQAPGQGLEWMGR
INPNSGGTNYAQKFQGRVTMTRDTSISTAYMELSRLRSDDTAVYYCASPR
GRGSAFDIWGQGTLVTVSSGGGGSGGGGSGGGASSYELTQPPSVSVSPGQ
TATISCSGDQLGGKYGHWYQKKPGQSPVLVLYQDRKRPAGIPERFSGSSS
GNTITLTISGTQAVDEADYYCQAWDTNLGGVFGGGTKVTVLGQPAA
SEQ ID No. 25
(CP_03_H06)
QVQLVESGGGLVKPGGSLRLSCAASGFTFSDYYMSWIRQAPGKGLEWVSY
ISSSGSTIYYADSVEGRFTISRDNAKNSLYLQMNSLRTEDTAVYYCARAR
VGGMDVWGQGTMVTVSSGGGGSGGGGSGGGASNFMLTQPHSVSESPGKTV
TISCTRSSGSIASNYVQWYQQRPGSSPTTVIYEDNQRPSGVPDRFSGSID
SSSNSASLTISGLKTEDEADYYCQSFDADNLHVVFGGGTKLTVLGQPAA
SEQ ID No. 26
(CP_03_C12)
EVQLVQSGAEVKKPGASVKVSCKASGYTFTGYYMHWVRQAPGQGLEWMGW
INPNSGGTNYAQKFQGRVTMTRDTSISTAYMELSSLRSEDTAVYYCARDT
GPIDYWGQGTMVTVSSGGGGSGGGGSGGGASDIVMTQTPLSLSVTPGQPA
SISCKSSQSLLHSDGKTYLYWYLQKPGQPPQLLVYEVSNRFSGVPDKFSG
SGSGTDFTLKISRVEAEDVGVYYCMQGIQLPPTFGGGTKVDIKRTAAA
SEQ ID No. 27
(CP_03_G02)
EVQLVQSGAEVKKPGSSVKVSCKASGGTFSSYAISWVRQAPGQGLEWMGI
INPSGGSTSYAQKFQGRVTMTRDTSTSIVYMELSSLRSEDTAVYYCARGV
WNSGSYLGFDYWGQGTLVTVSSGGGGSGGGGSGGGASDIQMTQSPSSLSA
SVGDRVTITCQASQDISNYLNWYQQKPGKAPKLLIYAASSLQSGVPSRFS
GSGSGTDFTLTISSLQPEDFATYYCQQSYSTPLTFGGGTKLEIKRTAAA
SEQ ID No. 28
(CP_03_D04)
EVQLVESGGGVVQPGRSLRLSCAASGFTFSSYAMHWVRQAPGKGLEWVAV
ISYDGSNKYYADSVKGRFTISRDNSKNTLYLQMNSLRAEDTAVYYCARGG
FTVPGGAFDIWGQGTLVTVSSGGGGSGGGGSGGGASNFMLTQPHSVSDSP
GKTVTISCTRSSGRIGSNFVQWYQQRPGSSPTTVIYEDDQRPSGVPARFS
GSIDSSSNSASLTISGLTTADEAGYYCQSYDASNVIFGGGTKLTVLGQPA
A
SEQ ID No. 29
(CP_03_F10)
EVQLVESGAEVKKPGASVKVSCKASGYTFTGYYMHWVRQAPGQGLEWMGR
INPNSGGTNYAQKFQGRVTMTRDTSISTAYMELSSLRSEDTAVYYCARFG
ERYAFDIWGQGTLVTVSSGGGGSGGGGSGGGASQSELTQPPSASGSPGQS
VTISCTGTSTDVGAFHFVSWYQHTPGKAPKLLISEVRKRASGVPDRFSGS
RSGNTASLTVSGLQSEDEADYFCSAYTGSNYVFGSGTKLTVLGQPAA
SEQ ID No. 30
(CP_03_G09)
QVQLQQSGPGLVKPSQTLSLTCAISGDSVSSNSAAWNWIRQSPSRGLEWL
GRTYYRSKWYNDYAVSVKSRITINPDTSKNQFSLQLNSVTPEDTAVYYCA
RDQWLANYYYYGMDVWGQGTLVTVSSGGGGSGGGGSGGGASSYELTQPLS
VSVALGQTARITCGGNNIGSKNVHWYQQKPGQAPVLVIYRDNNRPSGIPE
RFSGSNSGNTATLTISKAQAGDEADYYCQVWDSNSWVFGGGTKLTVLGQP
AA
SEQ ID No. 31
(CP_03_F09)
QMQLVQSGAEVKKPGASVKVSCKASGYTFASYYMHWVRQAPGQGLEWMGI
INPSGGSTSYAQKFQGRVTMTRDTSTSTVYMELSRLRSDDTAVYYCASNR
GGSYKSVGMDVWGQGTTVTVSSGGGGSGGGGSGGGASNFMLTQPQSVSES
PGKTVTISCTRSSGNFASKYVQWYQQRPGSSPTTVIYENYQRPSGVPDRF
SGSIDSSSNSATLTISGLKTEDEADYYCQSYDEVSVVFGGGTQLTVLGQP
AA
SEQ ID No. 32
(CP_03_D09)
EVQLVQSGAEVKKPGSSVKVSCEASGYTFTSYAISWVRQAPGQGLEWMGW
MNPNSGNTGYAQKFQGRVTMTRNTSISTAYMELSSLRSEDTAVYYCARVS
SYYGMDVWGQGTLVTVSSGGGGSGGGGSGGGASNFMLTQPLSVSESPGKT
VTISCTRSSGSIASNYVQWYQQRPGSAPTTVIYEDNQRPSGVPDRFSGSI
DSSSNSASLTISGLKTEDEADYYCQSYNSSNHWVFGGGTKVTVLGQPAA

Variants of the above amino acid sequences may also be used in the present invention, provided that the resulting antibody binds TRBC1 or TRBC2 and does not significantly cross-react. Typically such variants have a high degree of sequence identity with one of the sequences specified above.

Methods of alignment of sequences for comparison are well known in the art.

The NCBI Basic Local Alignment Search Tool (BLAST) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of the VL or VH domain or scFv typically have at least about 75%, for example at least about 80%, 90%, 95%, 96%, 97%, 98% or 99% sequence identity with the sequences given as SEQ ID Nos 1-3, 13-32.

Typically variants may contain one or more conservative amino acid substitutions compared to the original amino acid or nucleic acid sequence. Conservative substitutions are those substitutions that do not substantially affect or decrease the affinity of an antibody to bind TRBC1 or TRBC2. For example, a human antibody that specifically binds TRBC1 or TRBC2 may include up to 1, up to 2, up to 5, up to 10, or up to 15 conservative substitutions in either or both of the VH or VL compared to any of the sequences given as SEQ ID No. 1-3 or 13-32 and retain specific binding to TRBC1 or TRBC2.

Functionally similar amino acids which may be exchanged by way of conservative substitution are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

Preparation of Antibodies

Preparation of antibodies may be performed using standard laboratory techniques. Antibodies may be obtained from animal serum, or, in the case of monoclonal antibodies or fragments thereof, produced in cell culture. Recombinant DNA technology may be used to produce the antibodies according to established procedure, in bacterial or mammalian cell culture.

Methods for the production of monoclonal antibodies are well known in the art. Briefly, monoclonal antibodies are typically made by fusing myeloma cells with the spleen cells from a mouse or rabbit that has been immunized with the desired antigen. Herein, the desired antigen is TRBC1 or TRBC2 peptide, or a TCRβ chain comprising either TRBC1 or TRBC2.

Alternatively, antibodies and related molecules, particularly scFvs, may be made outside the immune system by combining libraries of VH and VL chains in a recombinant manner. Such libraries may be constructed and screened using phage-display technology as described in Example 12.

Identification of TRBC1/TRBC2 Selective Antibodies

Antibodies which are selective for either TRBC1 or TRBC2 may be identified using methods which are standard in the art. Methods for determining the binding specificity of an antibody include, but are not limited to, ELISA, western blot, immunohistochemistry, flow cytometry, Forster resonance energy transfer (FRET), phage display libraries, yeast two-hybrid screens, co-immunoprecipitation, bimolecular fluorescence complementation and tandem affinity purification.

To identify an antibody which is selective for either TRBC1 or TRBC2 the binding of the antibody to each of TRBC1 and TRBC2 is assessed. Typically, this is assessed by determining the binding of the antibody to each TRBC separately. An antibody which is selective binds to either TRBC1 or TRBC2 without significant binding to the other TRBC.

Antibody Mimetics

The agent may alternatively be a molecule which is not derived from or based on an immunoglobulin. A number of “antibody mimetic” designed repeat proteins (DRPs) have been developed to exploit the binding abilities of non-antibody polypeptides.

Repeat proteins such as ankyrin or leucine-rich repeat proteins are ubiquitous binding molecules which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. DARPins (Designed Ankyrin Repeat Proteins) are one example of an antibody mimetic based on this technology.

For Anticalins, the binding specificity is derived from lipocalins, a family of proteins which perform a range of functions in vivo associated with physiological transport and storage of chemically sensitive or insoluble compounds. Lipocalins have a robust intrinsic structure comprising a highly conserved β-barrel which supports four loops at one terminus of the protein. These loops for the entrance to a binding pocket and conformational differences in this part of the molecule account for the variation in binding specificity between different lipocalins.

Avimers are evolved from a large family of human extracellular receptor domainsby in vitro exon shuffling and phage display, generating multi-domain proteins with binding and inhibitory properties.

Versabodies are small proteins of 3-5 kDa with >15% cysteines which form a high disulfide density scaffold, replacing the hydrophobic core present in most proteins. The replacement of a large number of hydrophobic amino acids, comprising the hydrophobic core, with a small number of disulphides results in a protein that is smaller, more hydrophilic, more resistant to proteases and heat and has a lower density of T-cell epitopes. All four of these properties result in a protein having considerably reduced immunogenicity. They may also be manufactured in E. coli, and are highly soluble and stable.

Conjugates

The antibody or mimetic may be a conjugate of the antibody or mimetic and another agent or antibody, for example the conjugate may be a detectable entity or a chemotherapeutic entity.

The detectable entity may be a fluorescent moiety, for example a fluorescent peptide. A “fluorescent peptide” refers to a polypeptide which, following excitation, emits light at a detectable wavelength. Examples of fluorescent proteins include, but are not limited to, fluorescein isothiocyanate (FITC), phycoerythrin (PE), allophycocyanin (APC), green fluorescent protein (GFP), enhanced GFP, red fluorescent protein (RFP), blue fluorescent protein (BFP) and mCherry.

A selective TRBC1 or TRBC2 agent conjugated to a detectable entity may be used to determine the TRBC of a malignant T cell.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, nitrosoureas, ethylenimines/methylmelamine, alkyl sulfonates, antimetabolites, pyrimidine analogs, epipodophylotoxins, enzymes such as L-asparaginase; biological response modifiers such as IFNα, IL-2, G-CSF and GM-CSF; platinium coordination complexes such as cisplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

A TRBC selective agent conjugated to a chemotherapeutic entity enables the targeted delivery of the chemotherapeutic entity to cells which express either TRBC1 or TRBC2.

Bi-Specific T-Cell Engagers

A wide variety of molecules have been developed which are based on the basic concept of having two antibody-like binding domains.

Bispecific T-cell engaging molecules are a class of bispecific antibody-type molecules that have been developed, primarily for the use as anti-cancer drugs. They direct a host's immune system, more specifically the T cells' cytotoxic activity, against a target cell, such as a cancer cell. In these molecules, one binding domain binds to a T cell via the CD3 receptor, and the other to a target cells such as a tumor cell (via a tumor specific molecule). Since the bispecific molecule binds both the target cell and the T cell, it brings the target cell into proximity with the T cell, so that the T cell can exert its effect, for example, a cytotoxic effect on a cancer cell. The formation of the T cell:bispecific Ab:cancer cell complex induces signaling in the T cell leading to, for example, the release of cytotoxic mediators. Ideally, the agent only induces the desired signaling in the presence of the target cell, leading to selective killing.

Bispecific T-cell engaging molecules have been developed in a number of different formats, but one of the most common is a fusion consisting of two single-chain variable fragments (scFvs) of different antibodies. These are sometimes known as BiTEs (Bi-specific T-cell Engagers).

The agent used in the method of the present invention may be a bi-specific molecule which selectively recognises TRBC1 or TRBC2 and is capable of activating a T cell.

For example the agent may be a BiTE. The agent used in the method may comprise:
(i) a first domain which binds either TRBC1 or TRBC2; and
(ii) a second domain capable of activating a T cell.

The bi-specific molecule may comprise a signal peptide to aid in its production. The signal peptide may cause the bi-specific molecule to be secreted by a host cell, such that the bi-specific molecule can be harvested from the host cell supernatant.

The signal peptide may be at the amino terminus of the molecule. The bi-specific molecule may have the general formula: Signal peptide—first domain—second domain.

The bi-specific molecule may comprise a spacer sequence to connect the first domain with the second domain and spatially separate the two domains.

The spacer sequence may, for example, comprise an IgG1 hinge or a CD8 stalk. The linker may alternatively comprise an alternative linker sequence which has similar length and/or domain spacing properties as an IgG1 hinge or a CD8 stalk.

The bi-specific molecule may comprise JOVI-1, or a functional fragment thereof, as defined above.

Chimeric Antigen Receptor (CAR)

Chimeric antigen receptors (CARs), also known as chimeric T-cell receptors, artificial T-cell receptors and chimeric immunoreceptors, are engineered receptors, which graft an arbitrary specificity onto an immune effector cell. In a classical CAR, the specificity of a monoclonal antibody is grafted on to a T-cell. CAR-encoding nucleic acids may be transferred to T-cells using, for example, retroviral vectors. In this way, a large number of cancer-specific T-cells can be generated for adoptive cell transfer. Phase I clinical studies of this approach show efficacy.

The target-antigen binding domain of a CAR is commonly fused via a spacer and transmembrane domain to an endodomain, which comprises or associates with an intrcellular T-cell signalling domain. When the CAR binds the target-antigen, this results in the transmission of an activating signal to the T-cell it is expressed on.

The agent used in the method of the present invention may be a CAR which selectively recognises TRBC1 or TRBC2. The agent may be a T-cell which expresses a CAR which selectively recognises TRBC1 or TRBC2.

The CAR may also comprise a transmembrane domain which spans the membrane. It may comprise a hydrophobic alpha helix. The transmembrane domain may be derived from CD28, which gives good receptor stability.

The endodomain is the portion of the CAR involved in signal-transmission. The endodomain either comprises or associates with an intracellular T-cell signalling domain. After antigen recognition, receptors cluster and a signal is transmitted to the cell. The most commonly used T-cell signalling component is that of CD3-zeta which contains 3 ITAMs. This transmits an activation signal to the T-cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR may comprise the CD28 endodomain and OX40 and CD3-Zeta endodomain.

The CAR may comprise a signal peptide so that when the CAR is expressed inside a cell, such as a T-cell, the nascent protein is directed to the endoplasmic reticulum and subsequently to the cell surface, where it is expressed.

The CAR may comprise a spacer sequence to connect the TRBC-binding domain with the transmembrane domain and spatially separate the TRBC-binding domain from the endodomain. A flexible spacer allows to the TRBC-binding domain to orient in different directions to enable TRBC binding.

The spacer sequence may, for example, comprise an IgG1 Fc region, an IgG1 hinge or a CD8 stalk, or a combination thereof. The linker may alternatively comprise an alternative linker sequence which has similar length and/or domain spacing properties as an IgG1 Fc region, an IgG1 hinge or a CD8 stalk.

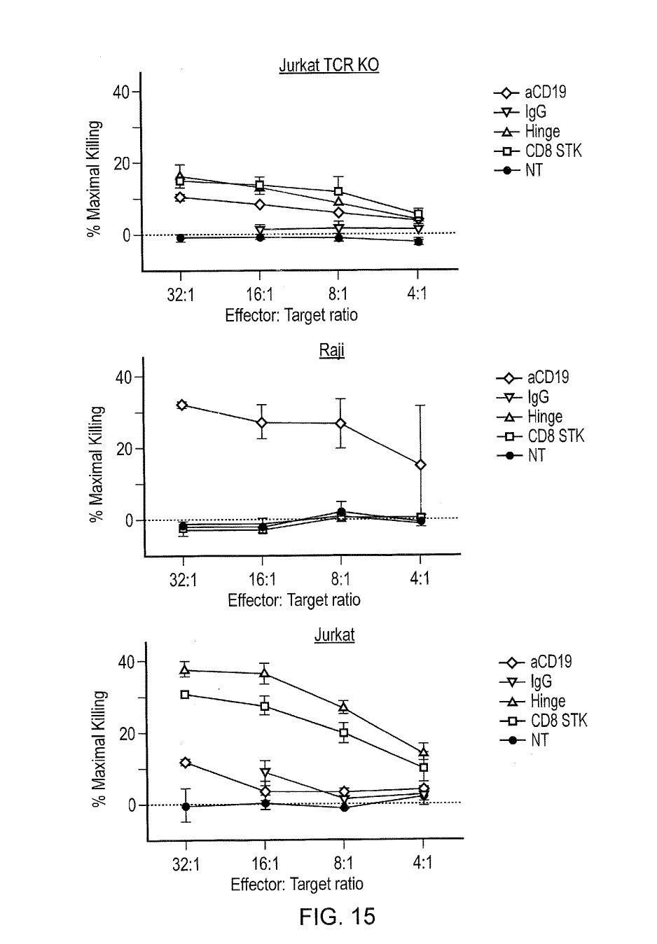

It was found that CARs comprising a spacer based on an IgG1 hinge or a CD8 stalk showed the best performance against Jurkat cells (FIG. 15). The spacer may an therefore comprise an IgG1 hinge or a CD8 stalk or a spacer which has a similar length and/or domain spacing properties as an IgG1 hinge or a CD8 stalk.

A human IgG1 spacer may be altered to remove Fc binding motifs.

The CAR may comprise the JOVI-1 antibody, or a functional fragment thereof, as defined above.

The CAR may comprise an amino acid sequence selected from the group consisting of SEQ ID No. 33, 34 and 35.

SEQ_ID_33
>JOVI-1 CAR with CD8 stalk spacer
METDTLLLWVLLVWIPGSTGEVRLQQSGPDLIKPGASVKMSCKASGYTFT
GYVMHWVKQRPGQGLEWIGFINPYNDDIQSNERFRGKATLTSDKSSTTAY
MELSSLTSEDSAVYYCARGAGYNFDGAYRFFDFWGQGTTLTVSSGGGGSG
GGGSGGGGSDVVMTQSPLSLPVSLGDQASISCRSSQRLVHSNGNTYLHWY
LQKPGQSPKLLIYRVSNRFPGVPDRFSGSGSGTDFTLKISRVEAEDLGIY
FCSQSTHVPYTFGGGTKLEIKRSDPTTTPAPRPPTPAPTIASQPLSLRPE
ACRPAAGGAVHTRGLDFACDIFWVLVVVGGVLACYSLLVTVAFIIFWVRS
KRSRLLHSDYMNMTPRRPGPTRKHYQPYAPPRDFAAYRSRDQRLPPDAHK
PPGGGSFRTPIQEEQADAHSTLAKIRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
SEQ_ID_34
>JOVI-1 CAR with H-CH2-CH3pvaa spacer
METDTLLLWVLLVWIPGSTGEVRLQQSGPDLIKPGASVKMSCKASGYTFT
GYVMHWVKQRPGQGLEWIGFINPYNDDIQSNERFRGKATLTSDKSSTTAY
MELSSLTSEDSAVYYCARGAGYNFDGAYRFFDFWGQGTTLTVSSGGGGSG
GGGSGGGGSDVVMTQSPLSLPVSLGDQASISCRSSQRLVHSNGNTYLHWY
LQKPGQSPKLLIYRVSNRFPGVPDRFSGSGSGTDFTLKISRVEAEDLGIY
FCSQSTHVPYTFGGGTKLEIKRSDPAEPKSPDKTHTCPPCPAPPVAGPSV
FLFPPKPKDTLMIARTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKTIPPVLDSDGSFFLYSKLTVDKSRWQQGNVFSCSVMHEALHNHYTQKS
LSLSPGKKDPKFWVLVVVGGVLACYSLLVTVAFIIFWVRSKRSRLLHSDY
MNMTPRRPGPTRKHYQPYAPPRDFAAYRSRDQRLPPDAHKPPGGGSFRTP
IQEEQADAHSTLAKIRVKFSRSADAPAYQQGQNQLYNELNLGRREEYDVL
DKRRGRDPEMGGKPRRKNPQEGLYNELQKDKMAEAYSEIGMKGERRRGKG
HDGLYQGLSTATKDTYDALHMQALPPR
SEQ_ID_35
>JOVI-1 CAR with IgG1 hinge spacer
METDILLLWVLLVWIPGSTGEVRLQQSGPDLIKPGASVKMSCKASGYTFT
GYVMHWVKQRPGQGLEWIGFINPYNDDIQSNERFRGKATLTSDKSSTTAY
MELSSLTSEDSAVYYCARGAGYNFDGAYRFFDFWGQGTTLTVSSGGGGSG
GGGSGGGGSDVVMTQSPLSLPVSLGDQASISCRSSQRLVHSNGNTYLHWY
LQKPGQSPKLLIYRVSNRFPGVPDRFSGSGSGTDFTLKISRVEAEDLGIY
FCSQSTHVPYTFGGGTKLEIKRSDPAEPKSPDKTHTCPPCPKDPKFWVLV
VVGGVLACYSLLVTVAFIIFWVRSKRSRLLHSDYMNMTPRRPGPTRKHYQ
PYAPPRDFAAYRSRDQRLPPDAHKPPGGGSFRTPIQEEQADAHSTLAKIR
VKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTY
DALHMQALPPR

In the CAR sequences given above, one or more of the 6 CDRs each independently may or may not comprise one or more amino acid mutations (eg substitutions) compared to the sequences given as SEQ ID No. 7 to 12, provided that the resultant CAR retains the ability to bind to TRBC1.

Variants of the above amino acid sequences may also be used in the present invention, provided that the resulting CAR binds TRBC1 or TRBC2 and does not significantly cross-react. Typically such variants have a high degree of sequence identity with one of the sequences given as SEQ ID No. 33, 34 or 35.

Variants of the CAR typically have at least about 75%, for example at least about 80%, 90%, 95%, 96%, 97%, 98% or 99% sequence identity with one of the sequences given as SEQ ID Nos 33, 34 and 35.

Nucleic Acid

The present invention further provides a nucleic acid encoding an agent such as a BiTE or CAR of the first aspect of the invention.

The nucleic acid sequence may encode a CAR comprising one of the amino acid sequences shown as SEQ ID No. 33, 34 and 35.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Vector

The present invention also provides a vector, or kit of vectors, which comprises one or more nucleic acid sequence(s) of the invention. Such a vector may be used to introduce the nucleic acid sequence(s) into a host cell so that it expresses a CAR according to the first aspect of the invention.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention also relates to a cell, such as an immune cell, comprising a CAR according to the first aspect of the invention.

The cell may comprise a nucleic acid or a vector of the present invention.

The cell may be a T-cell or a natural killer (NK) cell.

T cell may be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

T or NK cells expressing the a CAR according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells expressing a CAR according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, CAR cells are generated by introducing DNA or RNA coding for the CAR by one of many means including transduction with a viral vector, transfection with DNA or RNA.

The CAR cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding a CAR according to the first aspect of the invention, for example by treatment with an anti-CD3 monoclonal antibody.

The T or NK cell of the invention may be made by:
(i) isolation of a T or NK cell-containing sample from a subject or other sources listed above; and
(ii) transduction or transfection of the T or NK cells with a nucleic acid sequence(s) encoding a CAR of the invention.

The T or NK cells may then by purified, for example, selected on the basis of expression of the antigen-binding domain of the antigen-binding polypeptide.

The present invention also provides a kit which comprises a T or NK cell comprising a CAR according to the first aspect of the invention.

Pharmaceutical Composition

The present invention also relates to a pharmaceutical composition containing a plurality of cells expressing a CAR of the first aspect of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

T-Cell Lymphoma and/or Leukaemia

The present invention relates to agents, cells and methods for treating a T-cell lymphoma and/or leukaemia.

A method for treating a T-cell lymphoma and/or leukaemia relates to the therapeutic use of an agent. Herein the agent may be administered to a subject having an existing disease of T-cell lymphoma and/or leukaemia in order to lessen, reduce or improve at least one symptom associated with the disease and/or to slow down, reduce or block the progression of the disease.

The method of the present invention may be used for the treatment of any lymphoma and/or leukaemia associated with the clonal expansion of a cell expressing a T-cell receptor (TCR) comprising a β constant region. As such the present invention relates to a method for treating a disease which involves malignant T cells which express a TCR comprising a TRBC.

The method of the present invention may be used to treat a T-cell lymphoma in which the malignant T-cell expresses a TCR comprising a TRBC. ‘Lymphoma’ is used herein according to its standard meaning to refer to a cancer which typically develops in the lymph nodes, but may also affect the spleen, bone marrow, blood and other organs. Lymphoma typically presents as a solid tumour of lymphoid cells. The primary symptom associated with lymphoma is lymphadenopathy, although secondary (B) symptoms can include fever, night sweats, weight loss, loss of appetite, fatigue, respiratory distress and itching.

The method of the present invention may be used to treat a T-cell leukaemia in which the malignant T-cell expresses a TCR comprising a TRBC. ‘Leukaemia’ is used herein according to its standard meaning to refer to a cancer of the blood or bone marrow.

The following is an illustrative, non-exhaustive list of diseases which may be treated by the method of the present invention.

Peripheral T-Cell Lymphoma

Peripheral T-cell lymphomas are relatively uncommon lymphomas and account fewer than 10% of all non-Hodgkin lymphomas (NHL). However, they are associated with an aggressive clinical course and the causes and precise cellular origins of most T-cell lymphomas are still not well defined.

Lymphoma usually first presents as swelling in the neck, underarm or groin. Additional swelling may occur where other lymph nodes are located such as in the spleen. In general, enlarged lymph nodes can encroach on the space of blood vessels, nerves, or the stomach, leading to swollen arms and legs, to tingling and numbness, or to feelings of being full, respectively. Lymphoma symptoms also include nonspecific symptoms such as fever, chills, unexplained weight loss, night sweats, lethargy, and itching.

The WHO classification utilizes morphologic and immunophenotypic features in conjunction with clinical aspects and in some instances genetics to delineate a prognostically and therapeutically meaningful categorization for peripheral T-cell lymphomas (Swerdlow et al.; WHO classification of tumours of haematopoietic and lymphoid tissues. 4th ed.; Lyon: IARC Press; 2008). The anatomic localization of neoplastic T-cells parallels in part their proposed normal cellular counterparts and functions and as such T-cell lymphomas are associated with lymph nodes and peripheral blood. This approach allows for better understanding of some of the manifestations of the T-cell lymphomas, including their cellular distribution, some aspects of morphology and even associated clinical findings.

The most common of the T-cell lymphomas is peripheral T-cell lymphoma, not otherwise specified (PTCL-NOS) comprising 25% overall, followed by angioimmunoblastic T-cell lymphoma (AITL) (18.5%)

Peripheral T-Cell Lymphoma, not Otherwise Specified (PTCL-NOS)

PTCL-NOS comprises over 25% of all peripheral T-cell lymphomas and NK/T-cell lymphomas and is the most common subtype. It is determined by a diagnosis of exclusion, not corresponding to any of the specific mature T-cell lymphoma entities listed in the current WHO 2008. As such it is analogous to diffuse large B-cell lymphoma, not otherwise specified (DLBCL-NOS).

Most patients are adults with a median age of 60 and a male to female ratio 2:1. The majority of cases are nodal in origin, however, extranodal presentations occur in approximately 13% of patients and most commonly involve skin and gastrointestinal tract.

The cytologic spectrum is very broad, ranging from polymorphous to monomorphous. Three morphologically defined variants have been described, including lymphoepithelioid (Lennert) variant, T-zone variant and follicular variant. The lymphoepithelioid variant of PTCL contains abundant background epithelioid histiocytes and is commonly positive for CD8. It has been associated with a better prognosis. The follicular variant of PTCL-NOS is emerging as a potentially distinct clinicopathologic entity.

The majority of PTCL-NOS have a mature T-cell phenotype and most cases are CD4-positive. 75% of cases show variable loss of at least one pan T-cell marker (CD3, CD2, CD5 or CD7), with CD7 and CD5 being most often downregulated. CD30 and rarely CD15 can be expressed, with CD15 being an adverse prognostic feature. CD56 expression, although uncommon, also has negative prognostic impact. Additional adverse pathologic prognostic factors include a proliferation rate greater than 25% based on KI-67 expression, and presence of more than 70% transformed cells. Immunophenotypic analysis of these lymphomas has offered little insight into their biology.

Angioimmunoblastic T-Cell Lymphoma (AITL)

AITL is a systemic disease characterized by a polymorphous infiltrate involving lymph nodes, prominent high endothelial venules (HEV) and peri-vascular expansion of follicular dendritic cell (FDC) meshworks. AITL is considered as a de-novo T-cell lymphoma derived from αβ T-cells of follicular helper type (TFH), normally found in the germinal centres.

AITL is the second most common entity among peripheral T-cell lymphoma and NK/T-cell lymphomas, comprising about 18.5% of cases. It occurs in middle aged to elderly adults, with a median age of 65 years old, and an approximately equal incidence in males and females. Clinically, patients usually have advanced stage disease, with generalized lymphadenopathy, hepatosplenomegaly and prominent constitutional symptoms. Skin rash with associated pruritus is commonly present. There is often polyclonal hypergammaglobulinemia, associated with autoimmune phenomena.

Three different morphologic patterns are described in AITL. The early lesion of AITL (Pattern I) usually shows preserved architecture with characteristic hyperplastic follicles. The neoplastic proliferation is localized to the periphery of the follicles. In Pattern II the nodal architecture is partially effaced with retention of few regressed follicles. The subcapsular sinuses are preserved and even dilated. The paracortex contains arborizing HEV and there is a proliferation of FDC beyond the B-cell follicle. The neoplastic cells are small to medium in size, with minimal cytologic atypia. They often have clear to pale cytoplasm, and may show distincT-cell membranes. A polymorphous inflammatory background is usually evident.

Although AITL is a T-cell malignancy, there is a characteristic expansion of B-cells and plasma cells, which likely reflects the function of the neoplastic cells as TFH cells. Both EBV-positive and EBV-negative B-cells are present. Occasionally, the atypical B-cells may resemble Hodgkin/Reed-Sternberg-like cells morphologically and immunophenotypically, sometimes leading to a diagnostic confusion with that entity. The B-cell proliferation in AITL may be extensive and some patients develop secondary EBV-positive diffuse large B-cell lymphomas (DLBCL) or—more rarely—EBV-negative B-cell tumors, often with plasmacytic differentiation.

The neoplastic CD4-positive T-cells of AITL show strong expression of CD10 and CD279 (PD-1) and are positive for CXCL13. CXCL13 leads to an increased B-cell recruitment to lymph nodes via adherence to the HEV, B-cell activation, plasmacytic differentiation and expansion of the FDC meshworks, all contributing to the morphologic and clinical features of AITL. Intense PD-1-expression in the perifollicular tumor cells is particularly helpful in distinguishing AITL Pattern I from reactive follicular and paracortical hyperplasia.

The follicular variant of PTCL-NOS is another entity with a TFH phenotype. In contradistinction to AITL, it does not have prominent HEV or extra-follicular expansion of FDC meshworks. The neoplastic cells may form intrafollicular aggregates, mimicking B-cell follicular lymphoma, but also can have interfollicular growth pattern or involve expanded mantle zones. Clinically, the follicular variant of PTCL-NOS is distinct from AITL as patients more often present with early stage disease with partial lymph node involvement and may lack the constitutional symptoms associated with AITL.

Anaplastic Large Cell Lymphoma (ALCL)

ALCL may be subdivided as ALCL-‘anaplastic lymphoma kinase’ (ALK)+ or ALCL-ALK−.

ALCL-ALK+ is one of the best-defined entities within the peripheral T-cell lymphomas, with characteristic “hallmark cells” bearing horseshoe-shaped nuclei and expressing ALK and CD30. It accounts for about 7% of all peripheral T-cell and NK-cell lymphomas and is most common in the first three decades of life. Patients often present with lymphadenopathy, but the involvement of extranodal sites (skin, bone, soft tissues, lung, liver) and B symptoms is common.

ALCL, ALK+ shows a wide morphologic spectrum, with 5 different patterns described, but all variants contain some hallmark cells. Hallmark cells have eccentric horseshoe- or kidney-shaped nuclei, and a prominent perinuclear eosinophilic Golgi region. The tumour cells grow in a cohesive pattern with predilection for sinus involvement. Smaller tumour cells predominate in the small cell variant, and in the lymphohistiocytic variant abundant histiocytes mask the presence of tumour cells, many of which are small.

By definition, all cases show ALK and CD30 positivity, with expression usually weaker in the smaller tumour cells. There is often loss of pan-T-cell markers, with 75% of cases lacking surface expression of CD3.

ALK expression is a result of a characteristic recurrent genetic alteration consisting of a rearrangement of ALK gene on chromosome 2p23 to one of the many partner genes, resulting in an expression of chimeric protein. The most common partner gene, occurring in 75% of cases, is Nucleophosmin (NPM1) on chromosome 5q35, resulting in t(2;5)(p23;q35). The cellular distribution of ALK in different translocation variants may vary depending on the partner gene.

ALCL-ALK− is included as a provisional category in the 2008 WHO classification. It is defined as a CD30 positive T-cell lymphoma that is morphologically indistinguishable from ALCL-ALK+ with a cohesive growth pattern and presence of hallmark cells, but lacking ALK protein expression.

Patients are usually adults between the ages of 40 and 65, in contrast to ALCL-ALK+, which is more common in children and young adults. ALCL-ALK− can involve both lymph nodes and extranodal tissues, although the latter is seen less commonly than in ALCL-ALK+. Most cases of ALCL-ALK− demonstrate effacement of lymph node architecture by sheets of cohesive neoplastic cells with typical “hallmark” features. In contrast to the ALCL-ALK+, the small cell morphologic variant is not recognized.

Unlike its ALK+ counterpart, ALCL-ALK− shows a greater preservation of surface T-cell marker expression, while the expression of cytotoxic markers and epithelial membrane antigen (EMA) is less likely. Gene expression signatures and recurrent chromosomal imbalances are different in ALCL-ALK− and ALCL-ALK+, confirming that they are distinct entities at a molecular and genetic level.

ALCL-ALK− is clinically distinct from both ALCL-ALK+ and PTCL-NOS, with significant differences in prognosis among these three different entities. The 5 year overall survival of ALCL-ALK− is reported as 49% which is not as good as that of ALCL-ALK+ (at 70%), but at the same time it is significantly better than that of PTCL-NOS (32%).

Enteropathy-Associated T-Cell Lymphoma (EATL)

EATL is an aggressive neoplasm which thought to be derived from the intraepithelial T-cells of the intestine. Two morphologically, immunohistochemically and genetically distinct types of EATL are recognized in the 2008 WHO classification: Type I (representing the majority of EATL) and Type II (comprising 10-20% of cases).

Type I EATL is usually associated with overt or clinically silent gluten-sensitive enteropathy, and is more often seen in patients of Northern European extraction due to high prevalence of celiac disease in this population.

Most commonly, the lesions of EATL are found in the jejunum or ileum (90% of cases), with rare presentations in duodenum, colon, stomach, or areas outside of the gastrointestinal tract. The intestinal lesions are usually multifocal with mucosal ulceration. Clinical course of EATL is aggressive with most patients dying of disease or complications of disease within 1 year.

The cytological spectrum of EATL type I is broad, and some cases may contain anaplastic cells. There is a polymorphous inflammatory background, which may obscure the neoplastic component in some cases. The intestinal mucosa in regions adjacent to the tumour often shows features of celiac disease with blunting of the villi and increased numbers of intraepithelial lymphocytes (IEL), which may represent lesional precursor cells.

By immunohistochemistry, the neoplastic cells are often CD3+CD4-CD8-CD7+CD5-CD56-βF1+, and contain cytotoxic granule-associated proteins (TIA-1, granzyme B, perforin). CD30 is partially expressed in almost all cases. CD103, which is a mucosal homing receptor, can be expressed in EATL.

Type II EATL, also referred to as monomorphic CD56+ intestinal T-cell lymphoma, is defined as an intestinal tumour composed of small- to medium-sized monomorphic T-cells that express both CD8 and CD56. There is often a lateral spread of tumour within the mucosa, and absence of an inflammatory background. The majority of cases express the γδ TCR, however there are cases associated with the αβ TCR.

Type II EATL has a more world-wide distribution than Type I EATL and is often seen in Asians or Hispanic populations, in whom celiac disease is rare. In individuals of European descent EATL, II represents about 20% of intestinal T-cell lymphomas, with a history of celiac disease in at least a subset of cases. The clinical course is aggressive.

Hepatosplenic T-Cell Lymphoma (HSTL)

HSTL is an aggressive systemic neoplasm generally derived from γδ cytotoxic T-cells of the innate immune system, however, it may also be derived from αβ T-cells in rare cases. It is one of the rarest T-cell lymphomas, and typically affects adolescents and young adults (median age, 35 years) with a strong male predominance.

Extranodal NK/T-Cell Lymphoma Nasal Type

Extranodal NK/T-cell lymphoma, nasal type, is an aggressive disease, often with destructive midline lesions and necrosis. Most cases are of NK-cell derivation, but some cases are derived from cytotoxic T-cells. It is universally associated with Epstein-Barr Virus (EBV).

Cutaneous T-Cell Lymphoma

The method of the present invention may also be used to treat cutaneous T-cell lymphoma.

Cutaneous T-cell lymphoma (CTCL) is characterised by migration of malignant T-cells to the skin, which causes various lesions to appear. These lesions change shape as the disease progresses, typically beginning as what appears to be a rash and eventually forming plaques and tumours before metastasizing to other parts of the body.

Cutaneous T-cell lymphomas include those mentioned in the following illustrative, non-exhaustive list; mycosis fungoides, pagetoid reticulosis, Sézary syndrome, granulomatous slack skin, lymphomatoid papulosis, pityriasis lichenoides chronica, CD30+ cutaneous T-cell lymphoma, secondary cutaneous CD30+ large cell lymphoma, non-mycosis fungoides CD30− cutaneous large T-cell lymphoma, pleomorphic T-cell lymphoma, Lennert lymphoma, subcutaneous T-cell lymphoma and angiocentric lymphoma.

The signs and symptoms of CTCL vary depending on the specific disease, of which the two most common types are mycosis fungoides and Sézary syndrome. Classic mycosis fungoides is divided into three stages:

Patch (atrophic or nonatrophic): Nonspecific dermatitis, patches on lower trunk and buttocks; minimal/absent pruritus;

Plaque: Intensely pruritic plaques, lymphadenopathy; and

Tumor: Prone to ulceration

Sézary syndrome is defined by erythroderma and leukemia. Signs and symptoms include edematous skin, lymphadenopathy, palmar and/or plantar hyperkeratosis, alopecia, nail dystrophy, ectropion and hepatosplenomegaly.

Of all primary cutaneous lymphomas, 65% are of the T-cell type. The most common immunophenotype is CD4 positive. There is no common pathophysiology for these diseases, as the term cutaneous T-cell lymphoma encompasses a wide variety of disorders.

The primary etiologic mechanisms for the development of cutaneous T-cell lymphoma (ie, mycosis fungoides) have not been elucidated. Mycosis fungoides may be preceded by a T-cell-mediated chronic inflammatory skin disease, which may occasionally progress to a fatal lymphoma.

Primary Cutaneous ALCL (C-ALCL)

C-ALCL is often indistinguishable from ALC-ALK− by morphology. It is defined as a cutaneous tumour of large cells with anaplastic, pleomorphic or immunoblastic morphology with more than 75% of cells expressing CD30. Together with lymphomatoid papulosis (LyP), C-ALCL belongs to the spectrum of primary cutaneous CD30-positive T-cell lymphoproliferative disorders, which as a group comprise the second most common group of cutaneous T-cell lymphoproliferations after mycosis fungoides.

The immunohistochemical staining profile is quite similar to ALCL-ALK−, with a greater proportion of cases staining positive for cytotoxic markers. At least 75% of the tumour cells should be positive for CD30. CD15 may also be expressed, and when lymph node involvement occurs, the differential with classical Hodgkin lymphoma can be difficult. Rare cases of ALCL-ALK+ may present with localized cutaneous lesions, and may resemble C-ALCL.

T-Cell Acute Lymphoblastic Leukaemia

T-cell acute lymphoblastic leukaemia (T-ALL) accounts for about 15% and 25% of ALL in paediatric and adult cohorts respectively. Patients usually have high white blood cell counts and may present with organomegaly, particularly mediastinal enlargement and CNS involvement.

The method of the present invention may be used to treat T-ALL which is associated with a malignant T cell which expresses a TCR comprising a TRBC.

T-Cell Prolymphocytic Leukaemia

T-cell-prolymphocytic leukemia (T-PLL) is a mature T-cell leukaemia with aggressive behaviour and predilection for blood, bone marrow, lymph nodes, liver, spleen, and skin involvement. T-PLL primarily affects adults over the age of 30. Other names include T-cell chronic lymphocytic leukaemia, “knobby” type of T-cell leukaemia, and T-prolymphocytic leukaemia/T-cell lymphocytic leukaemia.

In the peripheral blood, T-PLL consists of medium-sized lymphocytes with single nucleoli and basophilic cytoplasm with occasional blebs or projections. The nuclei are usually round to oval in shape, with occasional patients having cells with a more irregular nuclear outline that is similar to the cerebriform nuclear shape seen in Sézary syndrome. A small cell variant comprises 20% of all T-PLL cases, and the Séezary cell-like (cerebriform) variant is seen in 5% of cases.

T-PLL has the immunophenotype of a mature (post-thymic) T-lymphocyte, and the neoplastic cells are typically positive for pan-T antigens CD2, CD3, and CD7 and negative for TdT and CD1a. The immunophenotype CD4+/CD8− is present in 60% of cases, the CD4+/CD8+ immunophenotype is present in 25%, and the CD4−/CD8+ immunophenotype is present in 15% of cases

Pharmaceutical Composition

The method of the present invention may comprise the step of administering the agent in the form of a pharmaceutical composition.

The agent may be administered with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s), and other carrier agents.

Administration

The administration of the agent can be accomplished using any of a variety of routes that make the active ingredient bioavailable. For example, the agent can be administered by oral and parenteral routes, intraperitoneally, intravenously, subcutaneously, transcutaneously, intramuscularly, via local delivery for example by catheter or stent.

Typically, a physician will determine the actual dosage which will be most suitable for an individual subject and it will vary with the age, weight and response of the particular patient. The dosage is such that it is sufficient to reduce or deplete the number of clonal T-cells expressing either TRBC1 or TRBC2.

Use

The present invention also provides an agent for use in treating a T-cell lymphoma according to the method of the first aspect. The agent may be any agent as defined above.

The present invention also relates to the use of an agent as defined above in the manufacture of a medicament for the treatment of a T-cell lymphoma according to the method of the first aspect.

Kit

The present invention further provides a kit comprising an agent as defined above for use in the treatment of a T-cell lymphoma according to the method of the first aspect.

The kit may also comprise a reagent(s) suitable for determining the TRBC of a malignant T-cell. For example the kit may comprise PCR primers or an antibody (antibodies) which are specific for either TRBC1 or TRBC2.

Method for Determining T-Cell Lymphoma and/or Leukaemia

The present invention further relates to a method for determining the presence of a T-cell lymphoma or leukaemia in a subject which comprises the step of determining the proportion of T-cells in a sample from a subject which are either TRBC1 or TRBC2 positive.

T-cell lymphomas involve the clonal expansion of individual malignant T-cells. As such the presence of a T-cell lymphoma in a subject may be identified by determining the proportion of either TRBC1 or TRBC2 T-cells in a sample derived from a patient.

The sample may be a peripheral blood sample, a lymph sample or a sample taken directly from a tumour e.g. a biopsy sample.

The proportion of total T-cells which are TRBC1 or TRBC2 positive which indicates the presence of a T-cell lymphoma or leukaemia may be, for example 80, 85, 90, 95, 98 or 99% of a total population of cells.

The method may involve determining infiltration by a distinct population of T-cells in a biopsy or a sample. Herein, the presence of a T-cell lymphoma or leukaemia is indicated where 80, 85, 90, 95, 98 or 99% of a total population of T cells in the sample are either TRBC1 or TRBC2.

The total T-cells in a sample may identified by determining the number of cells in the sample which express CD3, CD4, CD8 and/or CD45. A combination of these markers may also be used.

The proportion of total T cells in a sample which express either TRBC1 or TRBC2 may be determined using methods which are known in the art, for example flow cytometry, immunohistochemistry or fluorescent microscopy.

The invention will now be further described by way of Examples, which are meant to serve to assist one of ordinary skill in the art in carrying out the invention and are not intended in any way to limit the scope of the invention.

### EXAMPLES

### Example 1

### Discrimination of TRBC1 and TRBC2-Expressing Cells

The JOVI-1 antibody has been previously disclosed by Viney et al. (Hybridoma; 1992; 11(6); 701-713) and is available commercially (Abcam, ab5465). The present inventors determined that JOVI-1 is able to discriminate cells based on specific expression of TRBC1 or TRBC2.

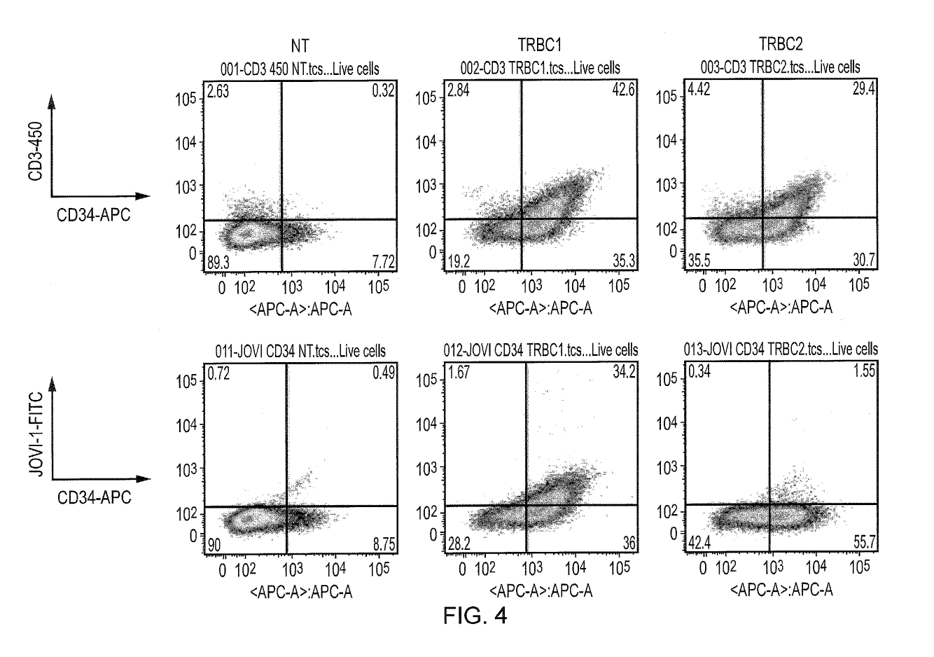

The inventors generated two plasmid vectors supplying the complete variable and constant regions of the TCR, differing only in expression of either TRBC1 or TRBC2. These plasmids were used to generate retroviral supernatant by transient transfection of 293T-cells. This supernatant was used to stably transduce Jurkats TCR-knockout T-cells (a T-ALL cell line with a mutation at the TCR beta chain locus precluding expression of this chain, and thereby the entire surface TCR/CD3 complex). This resulted in the production of cell lines which were identical other than expression of either TRBC1 or TRBC2. Staining of these cell lines revealed full expression of the surface TCR/CD3 complex, and that only cells expressing TRBC1 stained with the JOVI-1 antibody (FIG. 4).

### Example 2

### Normal Donor CD4+ and CD8+ T-Cells Contain Separate TRBC1-Positive and TRBC1-Negative Populations

The inventors tested the JOVI-1 antibody on primary human T-cells of normal donors.

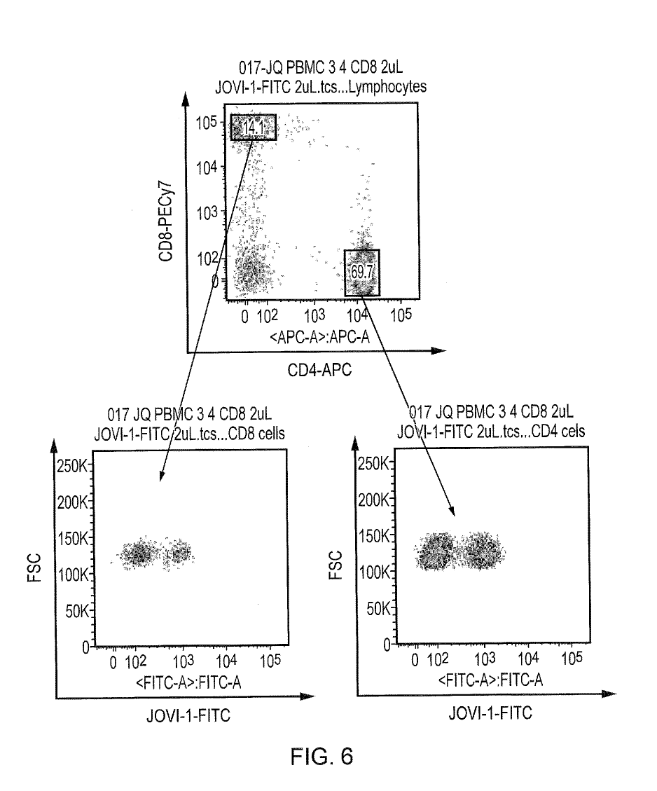

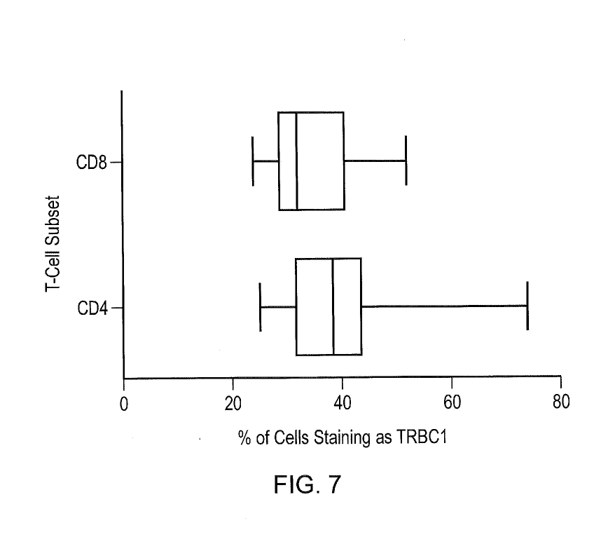

These analyses revealed that all donors had a proportion of both CD4+ and CD8+ T cells which expressed TRBC1 and a proportion of each which did not. Approximately 20-50% of normal CD4+ and CD8+ T-cells are TRBC1 +ve (FIGS. 6 and 7).

### Example 3

### Clonal T-Cell Lines Expressing TCR are TRBC1 Positive or Negative

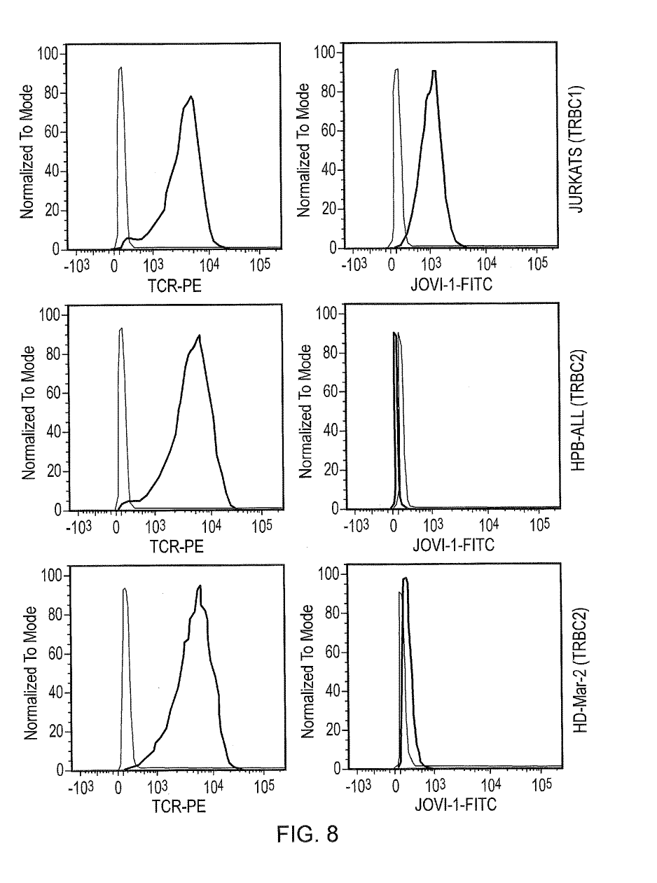

Cell lines are derived from an original clonal tumour population in a patient. Staining of T-cell lines expressing TCR reveals that T-cells express either TRBC1 or TRBC2, confirming this as a marker of clonality. Of three T-cell lines tested, Jurkats cells (known to be TRBC1+) and not HPB-ALL or HD-Mar-2 (known to be TRBC2+) cells stain with JOVI-1, supporting exclusive expression of either TRBC1 or 2 (FIG. 8).

### Example 4

### Primary Clonal T-Cell in Patients with T-Prolymphocyctic Leukaemia are TRBC1 Positive or Negative

Clonal T-cells extracted from peripheral blood of patients with T-prolymphocyctic leukaemia (T-PLL) are either uniformly TRBC1 positive or TRBC1 negative.

### Example 5

### The Effect of Mutation of the Residues that are Unique to TCBC1

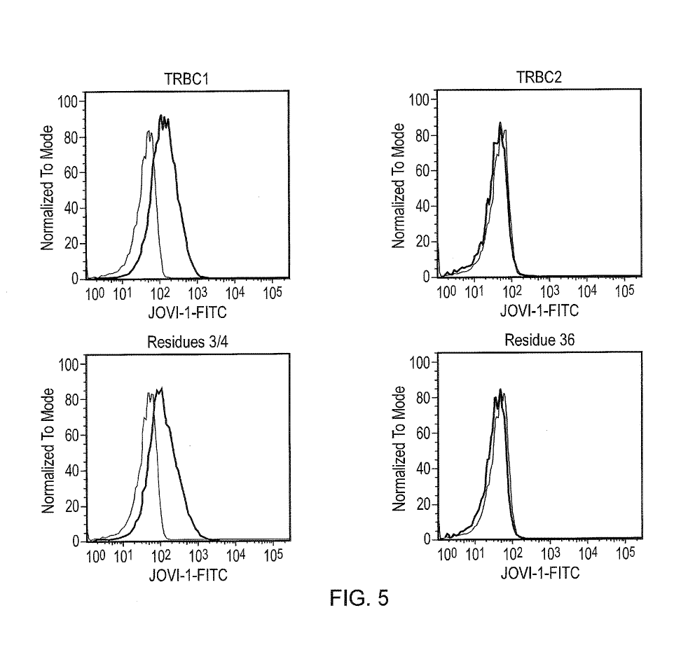

Plasmid vectors, coding for TCRs which are identical, except for hybrid TRBC1/2 mutations in the TCR 3 chain constant region, were generated. Analysis showed that JOVI-1 recognizes differences in residues at positions 3 and 4 of TCR p constant chain indicating that these residues are accessible to antibody recognition and are likely the best targets to generate agents discriminating TRBC1 from TRBC2 or TRBC2 from TRBC1 (FIG. 5).

### Example 6

### Specific Lysis of TRBC1 but not TRBC2 TCR Expressing T-Cells

Wild-type Jurkat T-cells (CD34−, TRBC1+) were mixed with TCRαβ knock-out Jurkat T-cells transduced with TRBC2 co-expressed with the CD34 marker gene (CD34+TRBC2+). These cells were incubated with JOVI-1 alone or incubated with JOVI-1 and complement for 1 hour. Cells were washed and stained for CD34, Annexin V and 7-AAD. Cells were analysed by flow-cytometry.

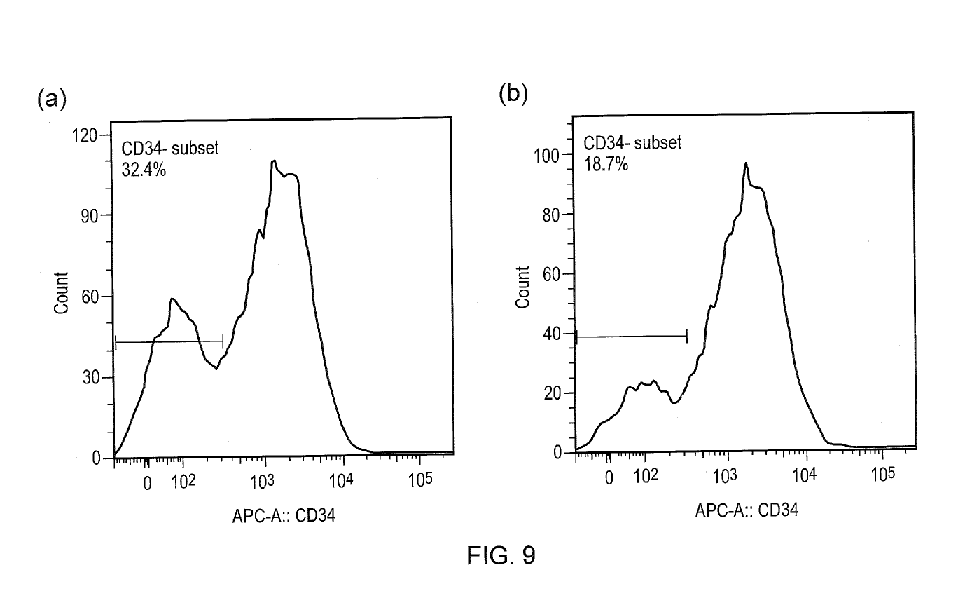

CD34 expression in the live population as defined by Annexin-V negative and 71AAD dim population is shown in FIG. 9. Selective killing of TRBC1 T-cells (CD34−) was observed (FIG. 9).

Wild-type Jurkat T-cells are naturally TRBC1+ and do not express the truncated CD34 marker gene. As described above the inventors derived a TRBC2+ Jurkat line by transducing TCRαβ knock-out Jurkat T-cells with a retroviral vector which codes for a TRBC2 TCR as well as the truncated CD34 marker gene. These T-cells were then mixed together. Next, the inventors incubated the T cells with either JOVI-1 alone or with JOVI-1 and complement for 1 hour. Conveniently, the inventors could discriminate TRBC1 and 2 populations by staining for the CD34 marker gene and thus avoided failing to detect TRBC1 TCRs due to TCR internalization after prolonged exposure to anti-TCR mAb. Cells were washed and stained for CD34, Annexin V and 7-AAD. The cells were analysed by flow-cytometry. By gating on live cells (i.e. cells which were Annexin V negative and 7-AAD dim), the inventors could determine that TRBC1 T-cells were selectively killed by JOVI-1 in the presence of complement (FIG. 9),

### Example 7

### Polyclonal Epstein Barr Virus (EBV) Specific T-Cells can be Split into Two Approximately Equal TRBC1/2 Populations

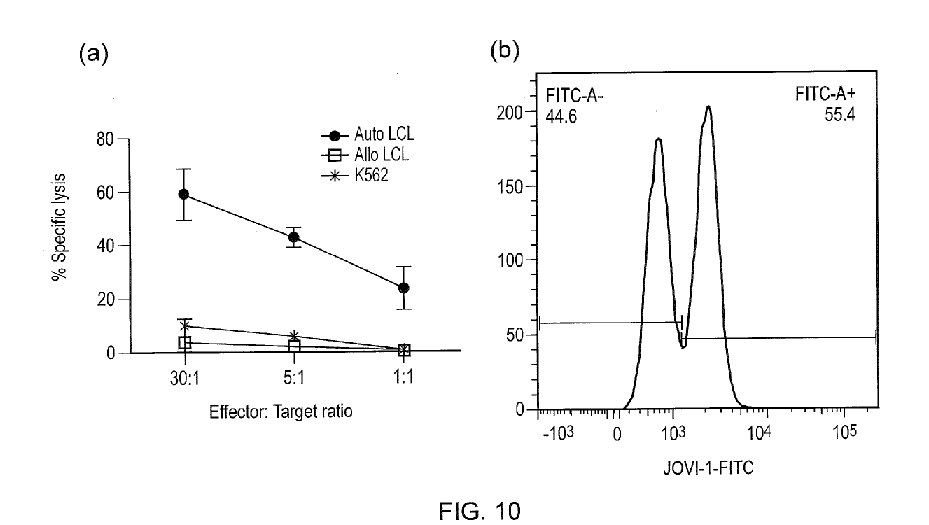

Peripheral blood T-cells were drawn from a normal blood donor. Mononuclear cells were isolated and most of the cells were cryopreserved. A small number of cells were infected with a laboratory strain of EBV (B95-8). Over some weeks, an immortalized EBV infected cell line, known as a lymphoblastoid cell line (LCL) emerged. Such a cell line is known to present a large collection of different EBV antigens. The previously cryopreserved mononuclear cells were thawed and repeatedly stimulated with this LCL line weekly for 4 weeks in the presence of IL2. This process selectively expands EBV specific T-cells from the peripheral blood mononuclear population. It is also known that such a process results in a polyclonal line where >90% of the T-cells are EBV specific and represents the donor's EBV immune system. The specificity of this line is checked by showing a high degree of killing of autologous LCLs but not allogeneic LCLs or K562 cells (FIG. 10a). This cell line was then stained with JOVI-1 and shown to contain an approximately equal mixture of TRBC1 and TRBC2 T-cells (FIG. 10b).

Thus, if a therapeutic agent was administered which depleted either the TRBC1 or TRBC2 compartment, an adequate EBV immunity would remain. Since EBV immunity is regarded as a model system for an immune response it is reasonable to postulate that immunity to other pathogens would be equally conserved.

### Example 8

### JOV1 Staining of a Circulating Peripheral T-Cell Lymphoma

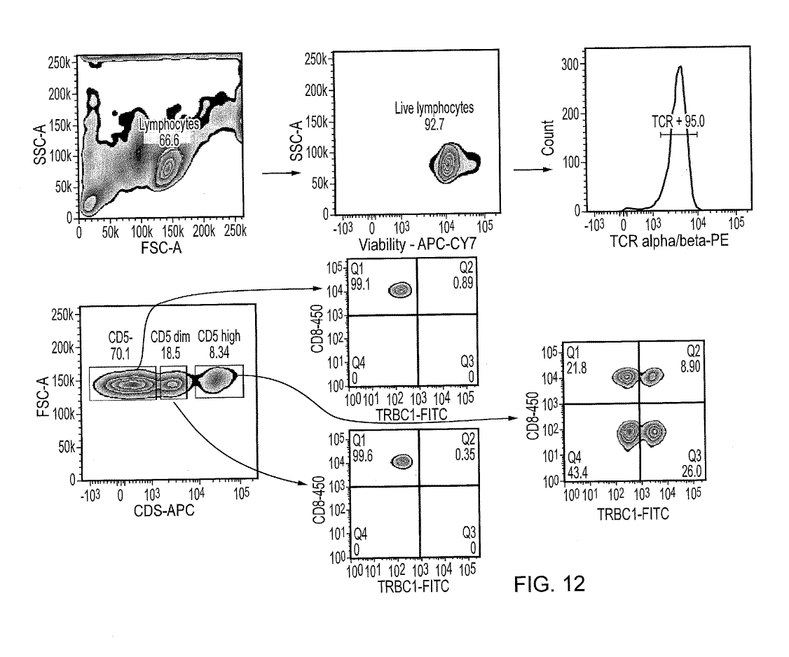

The hypothesis was that T-cell lymphomas, being clonal, would express either TRB1 or TRBC2 T-cell receptors, while normal T-cells being polyclonal would comprise of a population of T-cells which are a mixture of those that have TRBC1 or TRBC2. To demonstrate this, a blood sample of a T-cell lymphoma from a patient whose lymphoma was circulating in peripheral blood was obtained. Peripheral blood mononuclear cells were isolated and stained with a panel of antibodies which included CD5 and JOV11. The total T-cell population (which contains both lymphoma and normal T-cells) was first identified. This population was comprised of T-cells with normal (bright) CD5 expression and T-cells with intermediate/dim CD5 expression. The former represent normal T-cells, while the latter represent the lymphoma. JOVI-1 binding was investigated next and the results are shown in FIG. 12.

The CD5 intermediate and dim populations (the tumour) were all TRBC2 positive.

### Example 9

### Elucidation of the VHNL Sequences of JOVI-1

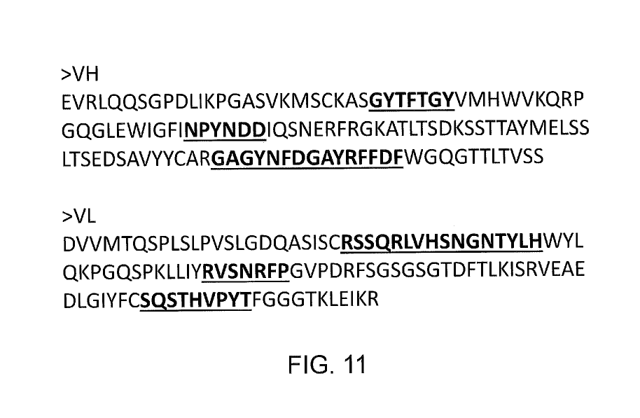

Using 5′ RACE with primers which anneal to the constant regions of mouse IgG CH1 and the constant region of mouse kappa, we isolated a single functional VH sequence and a single functional VL sequence from the hybridoma JOVI-1. Sequences of the VH and VL are SEQ ID 1 and 2 respectively (see above). An annotated sequence of VH and VL is shown in FIG. 11.

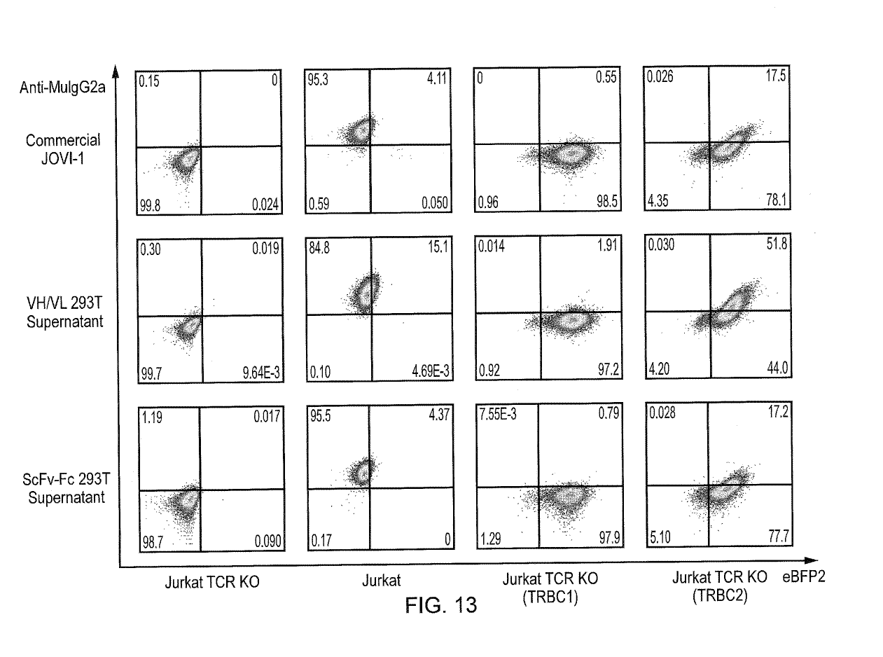

These VH and VL sequences were cloned back in frame with mouse IgG heavy chain and kappa light chain respectively. In addition, the VH and VL were fused to form a single-chain variable fragment (scFv), this was fused to the hinge-CH2-CH3 region of mouse IgG2a to create a scFv-Fv. The amino acid sequence of the scFv is given in the Detailed Description as SEQ ID No. 3. Recombinant antibody and recombinant scFv-Fc were generated by transfection into 293T cells. Along with JOVI-1 from the hybridoma, the following cells were stained: Jurkats with TCR knocked out; wild-type Jurkats; Jurkat TCR knock out transduced with TRBC1 co-expressed with eBFP2 and Jurkat TCR knock-out transduced with TRBC2 co-expressed with eBFP2. Both recombinant antibody and scFv-Fc derived from JOVI bound TRBC2 confirming that we had identified the correct VHNL, and that JOVI-1 VHNL can fold as a scFv. This binding data is shown in FIG. 13.

### Example 10

### Function of JOVI-1 Based CAR

The JOVI-1 scFv was cloned into CAR formats. To elucidate which spacer length would result in the optimal JOVI-1 based CAR, 3^{rd} generation CARs were generated with either a human Fc spacer, a human CD8 stalk spacer or with a spacer derived from an IgG1 hinge (FIG. 4). Primary human T-cells from normal donors were transduced with these CARs and killing of Jurkats and Jurkats with TCR knocked-out were compared. CARs with JOVI-1 scFvs which had either an IgG1 hinge spacer or a CD8 stalk spacer killed Jurkats, but not Jurkats with TCR knockout (FIG. 5), demonstrating the expected specificity. Since the normal donor T-cells transduced with the CARs should have a mixture of TRB1/2 T-cells, it was expected that the cultures would “self-purge”. Indeed, this was observed. JOVI-1 staining of the CAR T-cell cultures which become 100% TRBC2 negative in shown in FIG. 6.

Materials & Methods

Demonstration of Specificity of JOVI-1

A tri-cistronic retroviral cassette was generated which coded for a well characterized human TCR as well as a convenient marker gene. The coding sequences for the TCRα and β chains were generated using de-novo gene synthesis from overlapping oligonucleotides. These chains were connected in frame to a foot-and-mouth disease 2A peptide to allow co-expression. The truncated CD34 marker gene was cloned from cDNA by PCR and co-expressed with the TCR chains using an internal ribosome entry sequence (IRES). This cassette was introduced into a retroviral vector. Variants of this construct were generated by splice by over-lap PCR with primers which introduced the desired mutations. The veracity of the constructs was confirmed by Sanger sequencing. The Jurkat 76 line is a well characterized derivate of the Jurkat T-cell line which has both TCR α and β chains knocked out. This Jurkat line was transduced with the above retroviral vectors using standard techniques.

Staining and Analysis of Jurkats, Peripheral Blood T-Cells and Cell Lines

Jurkats were obtained from ECACC and engineered as detailed above. Other T-cell lines were also obtained from ECACC. Peripheral blood was drawn by venopunture from normal donors. Blood was ficolled to isolate mononuclear cells. Cells were stained with JOVI-1 as well as commercially available monoclonal antibodes which recognize all TCRs and CD3. In the case of engineered T-cells, cells were stained with antibodies which recognize CD34. In case of peripheral blood mononuclear cells, cells were stained with antibodies which recognize CD4 and CD8. The antibodies were purchased conjugated with suitable fluorophores so that independent fluorescent signals could be obtained while analyzing the cells with a flow-cytometer.

Demonstration of Specific Lysis of TRBC1 T-Cells

Wild-type Jurkat T-cells (TRBC1 − TCR), and Jurkat T-cells TCR KO with TRBC2 TCR introduced were mixed together at a ratio of 1:1. This mixture of Jurkats was then incubated with JOVI-1 monoclonal antibody at 1 ug/ml in the absence or presence of complement. Four hours later, cells were stained with Annexin-V and 7AAD and CD34. Conveniently, the marker gene CD34 can distinguish between wild-type (TRBC1) and transgenic (TRBC2) Jurkats. Cell populations were analysed by flow-cytometry. Live cells were selectively studied by gating on flow cytometric events which are negative for Annexin-V and dim for 7AAD. In this way, the survival of transgenic (TRBC2) vs wild-type (TRBC1) T-cells was studied.

### Example 11

### Investigating the Clonality of T-Cell Lymphoproliferative Disorders

Four patients: three with T-cell large granular lymphocyte lymphoproliferative disorder (T-LGL); and one with peripheral T-cell lymphoma (PCTL) were tested to confirm that malignant cells were uniformly either TRBC1 positive or negative.

Whole blood or bone marrow was collected from patients with T-lymphoproliferative orders. Peripheral blood mononuclear cells (PBMCs) were obtained by Ficoll gradient centrifugation. Freshly obtained PBMCs were pelleted and stained for 20 minutes with appropriate pre-conjugated antibodies. The cells were then washed and resuspended in phosphate buffered saline for immediate flow cytometric analysis on BD LSR Fortessa II. Live lymphocytes were identified by FSc/SSc properties and failure to uptake a dead cell discriminating dye. T-cells were identified by staining with anti-TCR alpha/beta antibody. Tumour and normal T-cell populations were identified using appropriate cell surface stains for each sample, based upon immunophenotype previously identified by clinical laboratory analysis.

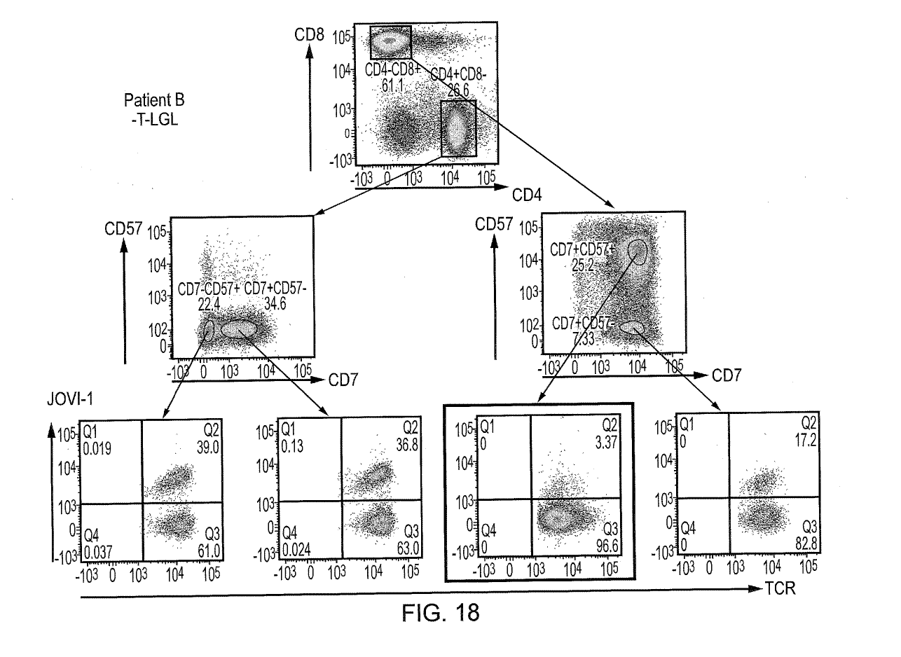

The results are shown in FIGS. 18 to 21.

In patient A (T-LGL, FIG. 18), normal T-cells were CD7bright and contained mixed CD4/CD8 cells, and a mixed population of TRBC1 or TRBC1− cells. In contrast, malignant cells were CD7− or CD7dim, were uniformly CD8+CD4−, and were uniformly TRBC1−.

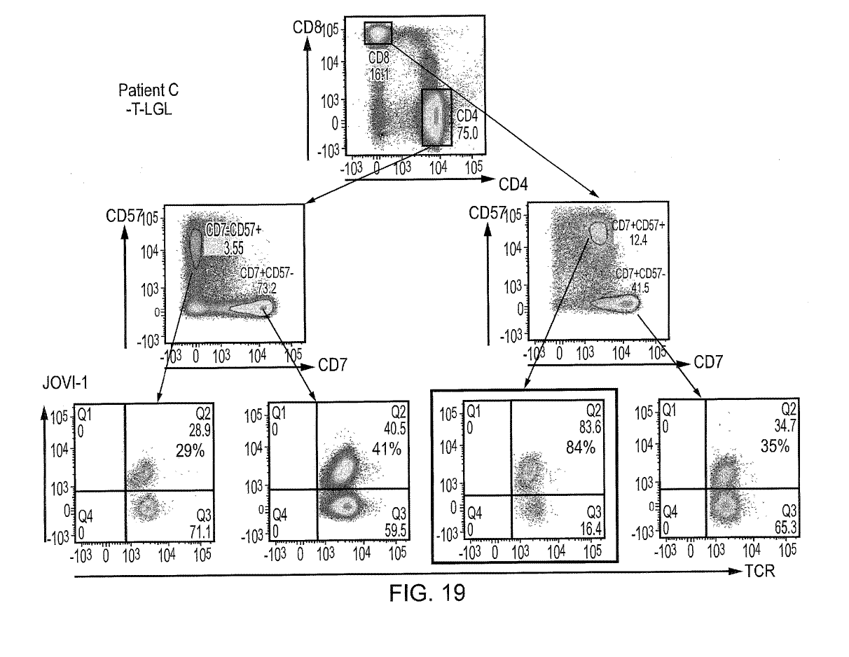

In patient B (T-LGL, FIG. 19), malignant cells were identified by CD4−, CD8+, CD7+ CD57+ and were clonally TRBC1− (highlighted panel). Normal CD4+CD8− and CD4− CD8+ T-cells contained TRBC1+ and TRBC1− populations.

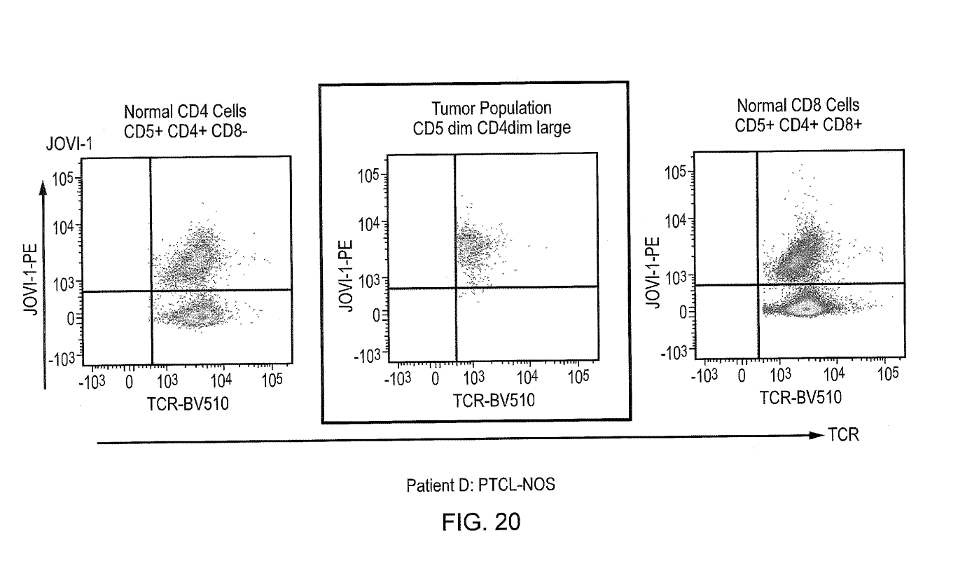

In patient C (T-LGL, FIG. 20), normal CD4+ and CD8+ T− cells populations were 30-40% TRBC1+. Malignant cells were identified by CD4−, CD8+, CD7+ CD57+ and were clonally TRBC1+ (highlighted panel, note 84% of cells are TRBC1—remaining 16% likely to be contaminating ‘normal’ T-cells). Normal CD4+CD8− and CD4−CD8+ T-cells contained TRBC1+ and TRBC1− populations.

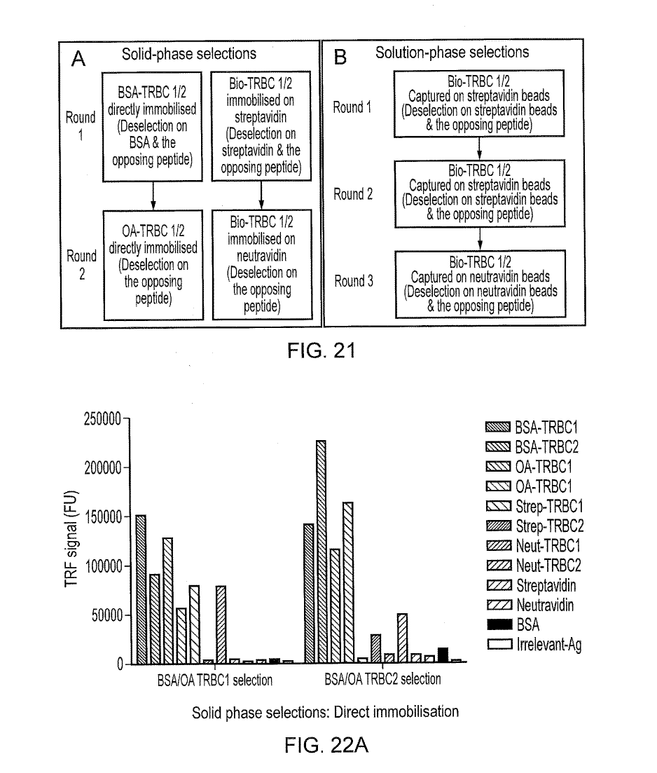

In patient D (PTCL-NOS, FIG. 21), malignant cells in the bone marrow, identified on the basis of FSChigh CD5dim CD4dim, were uniformly TRBC1+, whereas CD4+CD8− and CD4−CD8+ T-cells contained both TRBC1+ and TRBC1− populations.

### Example 12

### Generation of Monoclonal Human Antibodies which Distinguish Between the Two Isoforms of the T-Cell Receptor β Chain Constant Domain Using Phage Display

In order to generate antibodies which distinguished between TRBC2 and TRBC1 peptide fragments covering the region of difference between the two TRBC isoforms were synthesised. Of the four amino acid differences between TRBC2 and TRBC1, two are found at the beginning of the constant domains. Peptides (see below) representing these regions were synthesised and used for antibody generation.

(SEQ ID No. 36)
TRBC2 VLEDLKNVFPPEVAV
(SEQ ID No. 37)
TRBC1 VLEDLNKVFPPEVAV

These peptides were prepared in biotinylated, non-biotinylated and cysteine modified forms (by addition of a C terminal cysteine). The cysteine modified forms of TRBC1 and TRBC2 were subsequently conjugated to modified bovine serum albumin (Imm-Link BSA, Innova 462-001) or ovalbumin (Imm-Link Ovalbumin, Innova 461-001) according to manufacturers recommended conditions.

Results

Antibody Phage Display Selections

A human phage display library was constructed and phage selections carried out as described in as described in (Schofield et al., 2007 Genome Biol 8, R254). In order to identify TRBC1 and TRBC2 specific antibodies from the antibody library, multiple rounds of phage display selections were carried out. Two phage selection strategies were used in parallel to maximise the chance of generating a large panel of specific binders. These strategies are known as solid phase and solution phase selections (FIG. 21). In solid phase selections, phage antibodies are allowed to bind to the target antigen immobilised on a solid surface (Schofield et al., 2007, as above). In solution phase selections, phage antibodies binds to the biotinylated antigen in solution and the phage antibody-antigen complex is then captured by streptavidin or neutravidin coated paramagnetic beads. Within the solid phase selection strategy, two different immobilisation or antigen presentation approaches were employed. Using the first approach, TRBC peptides conjugated to bovine serum albumin (BSA) or ovalbumin (OA) were immobilised on Maxisorp™ immunotubes via direct adsorption. Using the second approach, biotinylated TRBC peptides were immobilised indirectly on Maxisorp™ immunotubes tubes that were pre-coated with streptavidin or neutravidin.

In order to select antibodies that are specific to the desired peptide, all selections were carried out in presence of an excess of the opposing peptide. For example, all TRBC1 selections were carried out in the presence of a 10-fold molar excess of non-biotinylated TRBC2. This method is known as ‘deselection’ and it was expected to deplete antibodies that recognise shared epitopes on both TRBC peptides as these preferentially bind to the excess TRBC2 in solution. In order to avoid enriching for antibody clones that bind to the carrier protein (BSA or OA) or the immobilisation partner (streptavidin or neutravidin from Thermo fisher scientific) two strategies were employed in combination.

The first strategy was to switch the conjugation or immobilisation partner between rounds of selection. For directly immobilised peptides the first round of selections were carried out on BSA-peptide and for round-2 OA-peptide conjugate was used. Similarly, biotinylated TRBC peptides were immobilised on streptavidin for the round-1 and neutravidin was used for immobilisation in round-2.

The second strategy was to deplete the phage library of any binders to the conjugation/immobilisation partner by performing a ‘deselection’ in round-1. For directly immobilised peptides, ‘deselection was performed by carrying out the phage-peptide binding step in the presence of 10-fold molar excess of free BSA in solution. In the case of biotinylated peptides immobilised on streptavidin, the phage library was pre-incubated with streptavidin coated paramagnetic beads. The beads were removed prior to the addition of the phage to antigen tubes thereby limiting the entry streptavidin binders into the selection. The different selection conditions used are summarised in FIG. 21. See Table 3 for detailed information on selection conditions.

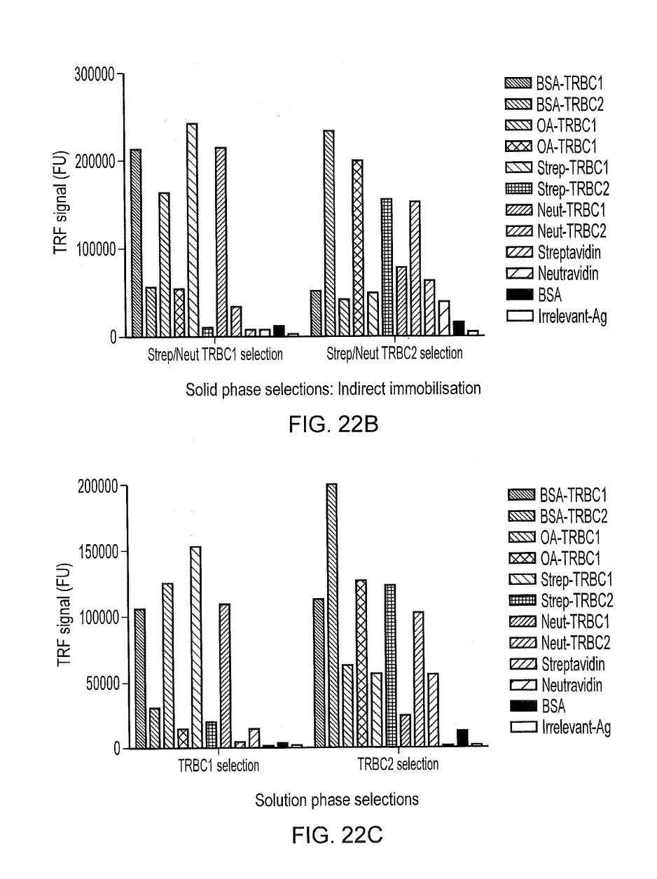

Polyclonal phage prepared from round-2 selection output was tested in ELISA using various presentations of the peptides or the support proteins. This included TRBC peptides directly immobilised as either BSA or OA conjugates or biotinylated peptides indirectly immobilised on streptavidin or neutravidin. Control proteins included were streptavidin, neutravidin, BSA and an irrelevant antigen. Phage binding was detected using a mouse anti-M13 antibody (GE healthcare) followed by an anti-mouse Fc antibody labelled with Europium (Perkin Elmers) using time resolved fluorescence (FIG. 22). This result demonstrated the preferential binding of polyclonal phage populations to the respective TRBC peptide (as compared to the opposing TRBC peptide). For example, polyclonal phage prepared from TRBC1 selections showed significantly higher binding signal to TRBC1 than TRBC2 and vice versa. There was limited or no binding to the immobilisation or conjugation partners and the irrelevant antigen.

Single Chain Antibody (scFv) Sub-Cloning and Monoclonal Screening

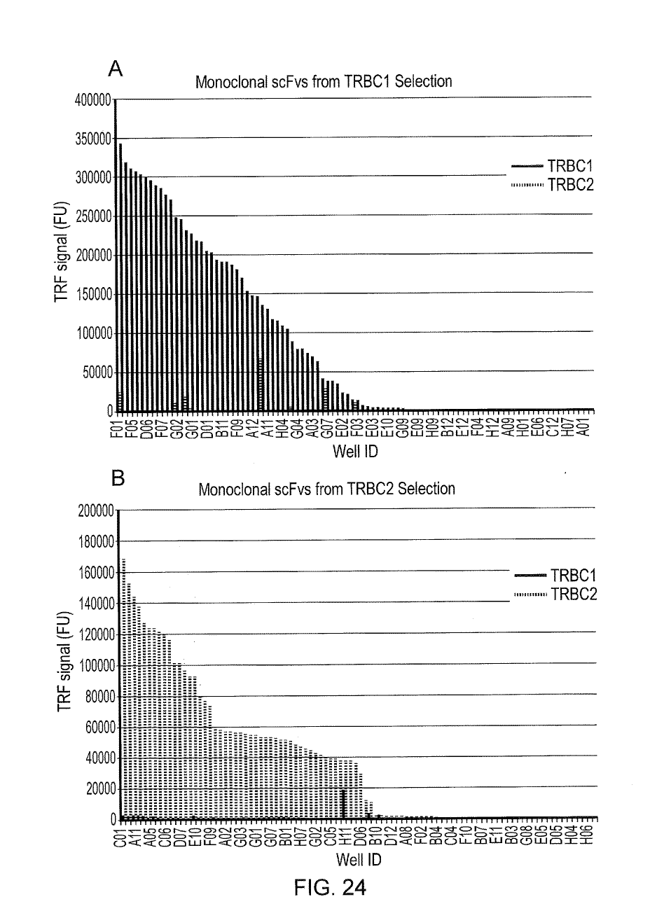

The scFv populations from round-2 and round-3 selection outputs were sub-cloned into the pSANG10-3F expression vector and transformed into E. coli BL21 (DE3) cells. 1128 individual transformants (564 clones/TRBC peptide) were picked into 12×96 well culture plates (94 clones/plate) and antibody expression was induced using autoinduction media. Recombinant monoclonal antibodies secreted into culture supernatant after overnight induction were tested for binding to biotinylated TRBC1 and TRBC2 immobilised on neutravidin coated Nunc Maxisorp™ 96 well plates. Out of the 564 clones screened from the TRBC1 selections, 255 clones were found to be specific for TRBC1 (>10000 TRF units for TRBC1 and <1000 TRF units for TRBC2). 138 TRBC2 specific binders (>10000 TRF units for TRBC2 and <2000 TRF units for TRBC1) were identified from the 564 clones screened from the TRBC2 selections. FIG. 24 shows a representative binding profile from a single 96 well plate arising from selection on either TRBC1 (FIG. 24A) or TRBC2 (FIG. 24B). The details of specific binders generated using different selection conditions is summarised in Table 5.

142 and 138 specific binders were picked from the TRBC1 and TRBC2 selections respectively for sequence analysis and further characterisation. Sequences of cherry-picked clones were generated by Sanger sequencing using Big Dye® terminator v3.1 cycle sequencing kit (Life technologies). DNA sequences were analysed to determine protein sequence and the CDRs of the VH and VL domains were identified. Analysis of the VH and VL CDR3 regions identified 74 unique TRBC1 and 42 unique TRBC2 clones (where unique is defined as any combination of VH CDR3 and VL CDR3 sequence). The TRBC1-specific clones and their VH CDR3 and VL CDR3 sequences are summarised in Table 1 above. The TRBC2-specific clones and their VH CDR3 and VL CDR3 sequences are summarised in Table 2 above.

TABLE 3A
Details of solid phase TRBC selections
Solid phase selections

| | No. | | | | Round 1 | Round 2 |
|---|---|---|---|---|---|---|
| Selection | of | Antigen | | | deselection | deselection |
| antigen | rounds | concentration | | Immobilisation | antigens | antigens |
| BSA TRBC1 | 2 | 10 | μg/ml | Direct | BSA (100 μg/ml), | TRBC2 |
| (Round 1), | | | | | TRBC2 (30 μM) | (30 μM) |
| OA TRBC1 | | | | | | |
| (Round 2) | | | | | | |
| BSA TRBC2 | 2 | 10 | μg/ml | Direct | BSA (100 μg/ml), | TRBC1 |
| (Round 1), | | | | | TRBC1 (30 μM) | (30 μM) |
| OA TRBC2 | | | | | | |
| (Round 2) | | | | | | |
| Bio-TRBC1 | 2 | 3 | μg/ml | Streptavidin | Streptavidin | Neutravidin |
| (Round 1&2) | | | | beads (Round | beads, TRBC2 | beads, |
| | | | | 1), Neutravidin | (30 μg/ml) | TRBC2 |
| | | | | (Round 2) | | (30 μg/ml) |
| Bio-TRBC2 | 2 | 3 | μg/ml | Streptavidin | Streptavidin | Neutravidin |
| (Round 1&2) | | | | beads (Round | beads, TRBC1 | beads, |
| | | | | 1), Neutravidin | (30 μg/ml) | TRBC1 |
| | | | | (Round 2) | | (30 μg/ml) |

TABLE 3B
Details of solution phase TRBC selections
Solution phase selections

| | No. | Antigen | Round 1 | Round 2 | Round 3 |
|---|---|---|---|---|---|
| Selection | of | concen- | deselection | deselection | deselection |
| antigen | rounds | tration | antigens | antigens | antigens |
| Bio- | 3 | 500 nM | Streptavidin | Streptavidin | Neutravidin |
| TRBC1 | | | beads, | beads, | beads, |
| (Round | | | TRBC2 | TRBC2 | TRBC2 |
| 1, 2&3) | | | (5 μM) | (5 μM) | (5 μM) |
| Bio- | 3 | 500 nM | Streptavidin | Streptavidin | Neutravidin |
| TRBC2 | | | beads, | beads, | beads, |
| (Round | | | TRBC1 | TRBC1 | TRBC1 |
| 1, 2&3) | | | (5 μM) | (5 μM) | (5 μM) |

TABLE 4
Selection output numbers

| | | | No. of | No. of | No. of |
|---|---|---|---|---|---|
| | | | plaque | plaque | plaque |
| | No. | | forming | forming | forming |
| Selection | of | | units | units | units |
| type | rounds | Antigen | (Round 1) | (Round 2) | (Round 3) |
| Solid phase | 2 | BSA/OA | 1.0 × 10⁴ | 6.0 × 10⁵ | N.D |
| | | TRBC1 | | | |
| Solid phase | 2 | BSA/OA | 1.5 × 10³ | 2.2 × 10⁶ | N.D |
| | | TRBC2 | | | |
| Solid phase | 2 | Bio-TRBC1 | 5.0 × 10³ | 2.0 × 10⁵ | N.D |
| Solid phase | 2 | Bio-TRBC2 | 3.0 × 10³ | 1.0 × 10⁴ | N.D |
| Solution | 3 | Bio-TRBC1 | 1.5 × 10⁶ | >10⁸ | >10⁸ |
| phase | | | | | |
| Solution | 3 | Bio-TRBC2 | 2.7 × 10⁵ | >10⁸ | >10⁸ |
| phase | | | | | |

TABLE 5
Details of monoclonal screening

| | | | | No. of | No. of |
|---|---|---|---|---|---|
| Selection | | Selection | Selection | clones | specific |
| number | Selection type | antigen | output | screened | binders |
| 262 | Solid-phase, | TRBC1 | Round-2 | 186 | 93 |
| | indirect | | | | |
| | immobilisation | | | | |
| 263 | Solid-phase, | TRBC2 | Round-2 | 186 | 68 |
| | indirect | | | | |
| | immobilisation | | | | |
| 264 | Solution-phase | TRBC1 | Round-2 | 186 | 83 |
| 265 | Solution-phase | TRBC2 | Round-2 | 186 | 29 |
| 266 | Solution-phase | TRBC1 | Round-3 | 94 | 47 |
| 267 | Solution-phase | TRBC2 | Round-3 | 94 | 33 |
| 268 | Solid-phase, | TRBC1 | Round-2 | 94 | 32 |
| | direct | | | | |
| | immobilisation | | | | |
| | (BSA/OA) | | | | |
| 269 | Solid-phase, | TRBC2 | Round-2 | 94 | 9 |
| | direct | | | | |
| | immobilisation | | | | |
| | (BSA/OA) | | | | |

### Example 13

### TRBC Polyclonal Antibody Production via Peptide Immunisation of Rabbits

In order to generate antibodies which distinguish between TRBC2 and TRBC1, 2 peptides that cover the principle area of differentiation between the two TRBC isoforms were synthesized and used for the immunization of rabbits. The following peptide sequences were used:

(SEQ ID No. 38)
TRBC1: VLEDLNKVFPPEVAVC
(SEQ ID No. 39)
TRBC2: VLEDLKNVFPPEVAVC.

15 mg of TRBC1 and TRBC2 peptides were synthesized. Keyhole Lympet Hemocyanin was conjugated to TRBC1 and TRBC2 peptides via C terminal cysteines present on the peptides. For each peptide 2 New England rabbits were immunized a total of three times with KLH conjugated TRBC1 or TRBC2 peptide. After the third immunization rabbits were sacrificed and bled, and the serum collected for purification. The crude serum obtained from the rabbits were passed through a crosslinked beaded agarose resin column coupled with the peptide used for immunization to collect antibodies specific for the common segments and the TRBC isoform specific epitope of the peptide. The initially purified supernatant was then purified further through a column with the alternative peptide immobilized, to remove the antibodies specific to common segments of the peptide.

ELISA Setup

Coating Antigen(s): A: peptide TRBC1
B: peptide TRBC2

Coating Concentration: 4 ug/ml, 100 μl/well

Coating Buffer: Phosphate Buffered Saline, pH7.4

Secondary Antibody: Anti-RABBIT IgG (H&L) (GOAT) Antibody Peroxidase Conjugated

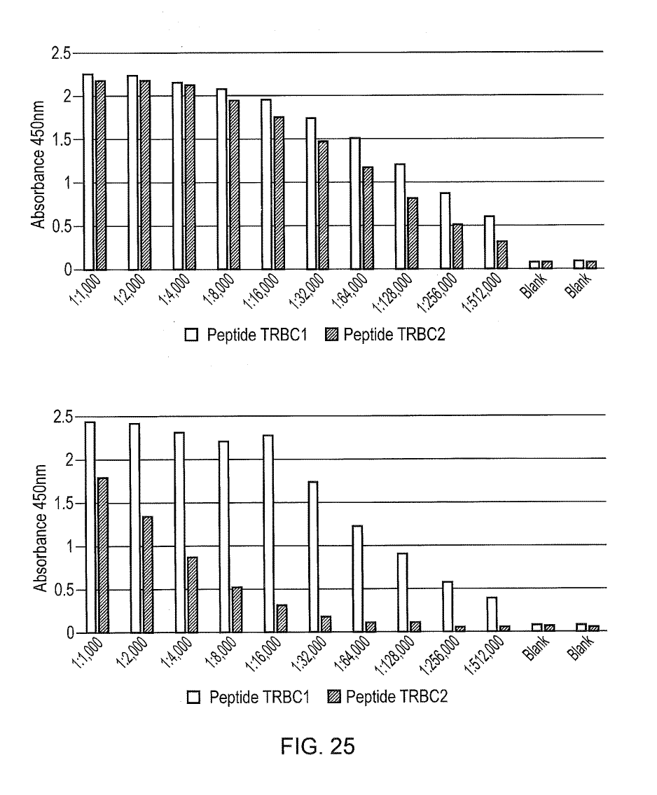

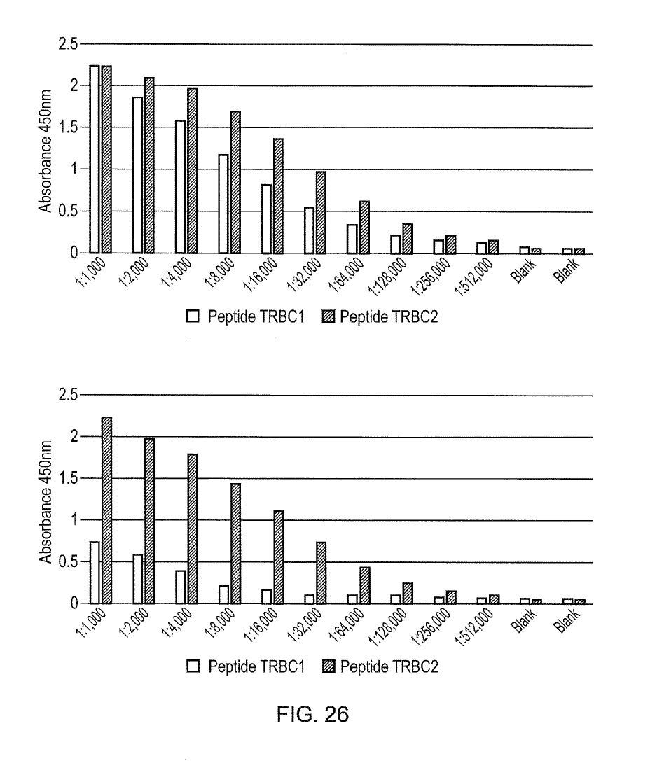

The results are shown in FIGS. 25 and 26. It is possible to make polyclonal serum comprising TRBC1 or TRBC2-specific antibodies by this method.

1. A chimeric antigen receptor (CAR) which comprises an antigen-binding domain which selectively binds TCR beta constant region 1 (TRBC1) or TRBC2.

2. A CAR according o claim 1 which selectively binds TRBC1.

3. A CAR according to claim 2, wherein the antigen-binding domain has a variable heavy chain (VH) and a variable light chain (VL) which comprise the following complementarity determining regions (CDRs):
VH CDR1: SEQ ID No. 7;
VH CDR2: SEQ ID No. 8;
VH CDR3: SEQ ID No. 9;
VL CDR1: SEQ ID No. 10;
VL CDR2: SEQ ID No. 11; and
VL CDR3: SEQ ID No. 12.

4. A CAR according to claim 2, wherein the antigen-binding domain comprises a variable heavy chain (VH) having the sequence shown as SEQ ID No. 1 and a variable light chain (VL) having the sequence shown as SEQ ID No. 2.

5. A CAR according to claim 2. wherein the antigen-binding domain comprises an scFv having the amino acid sequence shown as SEQ ID No. 3.

6. A CAR according o claim 2, which comprises an amino acid sequence selected from SEQ ID No. 4, 5 and 6

7. A CAR according to claim 1 which selectively binds TRBC2.

8. A nucleic acrid sequence encoding a CAR according to claim 1.

9. A vector which comprises a nucleic acid sequence according to claim 8.

10. A cell which comprises a CAR according to claim 1.

11. A T cell according to claim 10.

12. A method for making a cell according to claim 10 which comprises the step of transducing or transfecting a cell with a nucleic acid sequence according to claim 8 or a vector according to claim 9.

13. A method for treating a T-cell lymphoma or leukaemia in a subject which method comprises the step of
administrating a cell according to claim 10 comprising the TCRB1 or TCRB2 selective CAR to the subject, thereby causing selective depletion of the malignant T-cells together with normal T-cells expressing the same TRBC as the malignant T-cells, but not causing depletion of normal T-cells expressing the TRBC not expressed by the malignant T-cells.

14. A method according to claim 13, which also comprises the step of investigating the TCR beta constant region (TCRB) of a malignant T cell from the subject to determine whether it expresses TRBC1 or TRBC2.

15. A method according to claim 13, wherein the T-cell lymphoma or leukaemia is selected from peripheral T-cell lymphoma, not otherwise specified (PTCL-NOS); angio-immunoblastic T-cell lymphoma (AITL), anaplastic large cell lymphoma (AL(L), enteropathy-associated T-cell lymphoma (EATL), hepatosplenic T-cell lymphoma (HSTL), extranodal NKIT-cell lymphoma nasal type, cutaneous T-cell lymphoma, primary cutaneous ALCL, T cell prolymphocytic leukaemia and T-cell acute lymphoblastic leukaemia.

16. A method for diagnosing a T-cell lymphoma or leukaemia in a subject which comprises the step of determining the percentage of total T-cells in a sample isolated from the subject which are TRBC1 or TRBC2 positive.

17. A method according to claim 16 wherein a percentage of TRBC1 or TRBC2 positive T-cells which is greater than about 80% indicates the presence of a T-cell lymphoma or leukaemia.

18. A method according to claim 16 wherein the sample is a peripheral blood sample or a biopsy.

19. A method according to claim 16 wherein total T cells are identified in or isolated from the sample using an agent which binds CD3.
